(19) United States
(12) Patent Application Publication
(10) Pub. No.: US 2009/0053139 A1
(43) Pub. Date: Feb. 26, 2009

(54) Dendrimer based compositions and methods of using the same

(75) Inventors: Xiangyang Shi, Ann Arbor, MI (US); Suhe Wang, Ann Arbor, MI (US); James R. Baker, JR., Ann Arbor, MI (US)

Correspondence Address: Casimir Jones, S.C., 440 Science Drive, Suite 203, Madison, WI, 53711, US

(73) Assignee: Regents of the University of Michigan, Ann Arbor, MI (US)

(21) Appl. No.: 11/827,637

(22) Filed: Jul. 12, 2007

Related U.S. Application Data
(60) Provisional application No. 60/830,237, filed on Jul. 12, 2006.

(51) Int. Cl. A61K 49/00

(52) U.S. Cl. 424/9.1; 977/754

(57) ABSTRACT

The present invention relates to novel therapeutic and diagnostic dendrimers. In particular, the present invention is directed to dendrimer based compositions and systems for use in disease diagnosis and therapy (e.g., cancer diagnosis and therapy). The compositions and systems comprise one or more components for targeting, imaging, sensing, and/or providing a therapeutic or diagnostic material and monitoring the response to therapy of a cell or tissue (e.g., a tumor).

The present invention claims priority to U.S. Provisional Patent Application Ser. No. 60/830,237, filed Jun. 12, 2006, hereby incorporated by reference in its entirety.

This invention was made with government support under contract numbers EB002657, CO097111 and CA119409 awarded by the National Institutes of Health. The government has certain rights in the invention.

### FIELD OF THE INVENTION

The present invention relates to novel therapeutic and diagnostic dendrimers. In particular, the present invention is directed to dendrimer based compositions and systems for use in disease diagnosis and therapy (e.g., cancer diagnosis and therapy). The compositions and systems comprise one or more components for targeting, imaging, sensing, and/or providing a therapeutic or diagnostic material and monitoring the response to therapy of a cell or tissue (e.g., a tumor).

### BACKGROUND OF THE INVENTION

Cancer remains the number two cause of mortality in the United States, resulting in over 500,000 deaths per year. Despite advances in detection and treatment, cancer mortality remains high.

New compositions and methods for the imaging and treatment (e.g., therapeutic) of cancer (e.g., breast cancer, prostate cancer, colon cancer, pancreatic cancer, etc.) may help to reduce the rate of mortality associated with cancer. Such compositions and methods ideally should allow a physician to identify residual or minimal disease before and/or after treatment and/or to monitor response to therapy. This is important since a few remaining cells may result in re-growth, or worse, lead to a tumor that is resistant to therapy. Identifying residual disease at the end of therapy (i.e., rather than after tumor regrowth) may facilitate eradication of the few remaining tumor cells.

### SUMMARY OF THE INVENTION

The present invention relates to novel therapeutic and diagnostic dendrimers. In particular, the present invention is directed to dendrimer based compositions and systems for use in disease diagnosis and therapy (e.g., cancer diagnosis and therapy). The compositions and systems comprise one or more components for targeting, imaging, sensing, and/or providing a therapeutic or diagnostic material and monitoring the response to therapy of a cell or tissue (e.g., a tumor).

Accordingly, in some embodiments, the present invention provides a composition comprising a dendrimer and metal nanoparticles (e.g., superparamagnetic metal nanoparticles). In some embodiments, the metal nanoparticles are iron oxide nanoparticles. In some embodiments, the metal nanoparticles are poly(styrene sulfonate) (PSS)-coated iron oxide nanoparticles.

In some embodiments, the present invention provides a composition comprising dendrimers covalently linked to biopolymer-coated metal (e.g., iron oxide) nanoparticles. In some embodiments, the biopolymer-coated metal (e.g., iron oxide) nanoparticles are generated by a process comprising use of layer-by-layer self assembly of the biopolymer on the metal (e.g., iron oxide) nanoparticles. The present invention is not limited by the type of process comprising layer-by-layer assembly of the biopolymer on the metal (e.g., iron oxide) nanoparticles. Indeed, a variety of processes may be used including, but not limited to, sequentially providing a first polymer (e.g., positively charged polymer) comprising free amino groups to the nanoparticles, then providing a second polymer (e.g., a negatively charged polymer) comprising free carboxyl groups to the nanoparticles and allowing the layers to interact through electrostatic LbL assembly. The present invention is not limited by the type of biopolymer layer (e.g., comprising first and second polymers (e.g., assembled via layer-by-layer assembly)). Indeed, a variety of polymer pairs may be used to coat metal (e.g., iron oxide) nanoparticles including, but not limited to, hyaluronic acid and poly-arginine; alginate and chitosan; poly-lactic acid and polylysine; etc. In some embodiments, the polymer pair is poly(glutamic acid) (PGA) and poly-L-Lysine (PLL). In some embodiments, the hydroxyl groups of the metal (e.g., iron oxide) nanoparticles, the amino groups of the dendrimers, the carboxyl groups of the first polymer (e.g., PGA), and the amino groups of the second polymer (e.g., PLL) are covalently linked. The present invention is not limited by the type of process utilized for covalently linking (e.g., crosslinking) components of the composition. In some embodiments, the covalent attachments are formed by a process comprising crosslinking using 1-Ethyl-3-[3-dimethylaminopropyl]carbodiimide hydrochloride (EDC). The present invention is not limited to crosslinking with EDC chemistry. Indeed, a variety of methods can be used to crosslink NPs assembled with dendrimers including, but not limited to, Click chemistry, glutaraldehyde crosslinking, physical crosslinking (e.g., thermal crosslinking (e.g., to form amide bonds)), and/or UV irradiation crosslinking (e.g., of a carboxyl polymer assembled with a polycationic nitro-containing diazoresin). In some embodiments, the composition is subjected to a surface neutralization reaction. In some embodiments, the surface neutralization reaction decreases the surface charge of the dendrimers. The present invention is not limited by the type of surface modification reaction. Indeed, a variety of surface modification reactions (e.g., for decreasing the surface charge of the dendrimer (e.g., that decreases the zeta potential)) may be utilized. In some embodiments, the surface neutralization reaction comprises an acetylation reaction. In some embodiments, the surface neutralization reaction comprises reactions that add sugar and/or carbohydrates to the surface of the dendrimer, reactions that add small molecules to the surface of the dendrimer, and/or reactions that add any agent capable of surface neutralization (e.g., decreasing the surface charge of the dendrimer). In some embodiments, surface neutralization reactions comprise conjugation to a water soluble polymer (e.g., PEG). In some embodiments, the surface modification reaction (e.g., acetylation reaction) decreases the charge of unreacted amino groups of the dendrimers. In some embodiments, the dendrimer is a generation 5 (G5) polyamideamine (PAMAM) or polypropylamine (POPAM) dendrimer, although the present invention is not limited to any particular generation or chemistry used to generate the dendrimers. For example, in some embodiments, the dendrimer is a G3 dendrimer, a G4, dendrimer, a G5 dendrimer, a G6 dendrimer, a G7 dendrimer, a G8 denrimer, or a dendrimer of a generation greater than 8 or less than 3. In some embodiments, the composition comprises a dendron rather than or in addition to the dendrimer. In some embodiments, the dendrimer further comprises one or more functional groups, wherein the one or more functional groups are selected from the group consisting of a therapeutic agent, a targeting agent, an imaging agent, or a biological monitoring agent. In some embodiments, the therapeutic agent comprises a chemotherapeutic compound (e.g., methotrexate). In some embodiments, the chemotherapeutic compound is conjugated to the dendrimer via an ester bond, although the present invention is not so limited. In some embodiments, the a dendrimer of the composition comprises a targeting agent. The present invention is not limited by the type of targeting agent. Indeed, a number of targeting agents are contemplated to be useful in the present invention including, but not limited to, RGD sequences, low-density lipoprotein sequences, a NAALADase inhibitor, epidermal growth factor, and other agents that bind with specificity to a target cell (e.g., a cancer cell)). In some embodiments, the targeting agent comprises folic acid. The present invention is not limited by the number of agents attached to (e.g., conjugated to) the dendrimer. In some embodiments, the dendrimer comprises 1-3 agents (e.g., targeting agents). In some embodiments, the dendrimer comprises 3-4 agents. In some embodiments, the dendrimer comprises 5-10 agents. In some embodiments, the dendrimer comprises 10 or more agents (e.g., targeting agents conjugated to the dendrimer). In still other embodiments, the composition comprising a dendrimer and metal nanoparticles (e.g., iron oxide nanoparticles) further comprises a fluorescent agent (e.g., fluorescein isothiocyanate) or other detectable label. In some embodiments, the therapeutic agent (e.g., methotrexate) is conjugated to the dendrimer via an ester bond or an acid-labile linker. In some embodiments, the therapeutic agent is protected with a protecting group selected from photo-labile, radio-labile, and enzyme-labile protecting groups. In some embodiments, the composition is formed via charged interactions between the iron oxide nanoparticles and the dendrimer. In some embodiments, the composition is formed by incubating the dendrimer and iron oxide nanoparticles in a methanol solution containing acetic anhydride. In some embodiments, the metal nanoparticles (e.g., iron oxide nanoparticles) are conjugated to the dendrimer. In some embodiments, the conjugation comprises covalent bonds, ionic bonds, metallic bonds, hydrogen bonds, Van der Waals bonds, ester bonds or amide bonds. In some embodiments, the biopolymer-coated iron oxide nanoparticle are about 8.4 mm in diameter, although smaller and larger iron oxide nanoparticles may be used. In some embodiments, the therapeutic agent comprises a chemotherapeutic agent, an anti-oncogenic agent, an anti-angiogenic agent, a tumor suppressor agent, an anti-microbial agent, or an expression construct comprising a nucleic acid encoding a therapeutic protein. In some embodiments, the imaging agent comprises fluorescein isothiocyanate or 6-TAMARA.

The present invention also provides a method of imaging a cancer cell comprising providing a composition comprising dendrimers covalently linked to biopolymer-coated iron oxide nanoparticles, wherein the dendrimers comprise a targeting agent that binds with specificity to the cancer cell; and exposing the cancer cell to the composition under conditions such that the dendrimer interacts with and enters the cancer cell. In some embodiments, the targeting agent is folic acid. In some embodiments, the cancer cell expresses folic acid receptor. In some embodiments, the cancer cell is present in vivo. In some embodiments, the method images the cancer cell in a region beyond the primary tumor site. In some embodiments, detection of the cancer cell in a region beyond the primary tumor site is indicative of metastasis. In some embodiments, the imaging is used for staging of cancer. In some embodiments, the dendrimer further comprises a therapeutic agent. In some embodiments, the therapeutic agent is selected from the group comprising a chemotherapeutic agent, an anti-oncogenic agent, an anti-angiogenic agent, a tumor suppressor agent, an anti-microbial agent, and an expression construct comprising a nucleic acid encoding a therapeutic protein. In some embodiments, the dendrimer further comprises an imaging agent. In some embodiments, the imaging agent comprises fluorescein isothiocyanate.

The present invention also provides a method of generating a composition comprising dendrimers covalently linked to biopolymer-coated iron oxide nanoparticles comprising: providing: iron oxide nanoparticles; a pair of biocompatible polymers, wherein the first polymer comprises free amino groups and the second polymer comprises free carboxyl groups; and dendrimers; and allowing layer-by-layer assembly of the first polymer, the second polymer and the dendrimers to occur on the nanoparticles; crosslinking the layers, wherein the crosslinking comprises covalent attachment of hydroxyl groups of the nanoparticles, carboxyl groups of the second polymer, amino groups of the first polymer and amino groups of the dendrimers; and exposing the crosslinked layers to a surface neutralization reaction. In some embodiments, the first polymer is poly-L-Lysine (PLL). In some embodiments, the second polymer is poly(glutamic acid) (PGA). In some embodiments, the layers are crosslinked using 1-Ethyl-3-[3-dimethylaminopropyl]carbodiimide hydrochloride (EDC). In some embodiments, the neutralization reaction comprises an acetylation reaction. The present invention is not limited by the type of dendrimers utilized. Indeed, a variety of dendrimers may be utilized including those described herein (e.g., generation 5 (G5) polyamideamine (PAMAM) or polypropylamine (POPAM) dendrimers).

The present invention also provides a method of treating cancer comprising administering to a subject suffering from or susceptible to cancer a therapeutically effective amount of a composition comprising a dendrimer and metal nanoparticles (e.g., iron oxide nanoparticles (e.g., SCIO NPs). The present invention is not limited by the type of cancer treated using the compositions and methods of the present invention. Indeed, a variety of cancer can be treated including, but not limited to, prostate cancer, colon cancer, breast cancer, lung cancer and epithelial cancer.

The present invention also provides a kit comprising a composition comprising dendrimers covalently linked to biopolymer-coated iron oxide nanoparticles. In some embodiments, the kit comprises a fluorescent agent or bioluminescent agent.

In some embodiments of the present invention, the therapeutic agent includes, but is not limited to, a chemotherapeutic agent, an anti-oncogenic agent, an anti-angiogenic agent, a tumor suppressor agent, an anti-microbial agent, or an expression construct comprising a nucleic acid encoding a therapeutic protein, although the present invention is not limited by the nature of the therapeutic agent. In further embodiments, the therapeutic agent is protected with a protecting group selected from photo-labile, radio-labile, and enzyme-labile protecting groups. In some embodiments, the chemotherapeutic agent is selected from a group consisting of, but not limited to, platinum complex, verapamil, podophylltoxin, carboplatin, procarbazine, mechloroethamine, cyclophosphamide, camptothecin, ifosfamide, melphalan, chlorambucil, bisulfan, nitrosurea, adriamycin, dactinomycin, daunorubicin, doxorubicin, bleomycin, plicomycin, mitomycin, bleomycin, etoposide, tamoxifen, paclitaxel, taxol, transplatinum, 5-fluorouracil, vincristin, vinblastin, bisphosphonate (e.g., CB3717), chemotherapeutic agents with high affinity for folic acid receptors, ALIMTA (Eli Lilly), and methotrexate. In some embodiments, the anti-oncogenic agent comprises an antisense nucleic acid (e.g., RNA, molecule). In certain embodiments, the antisense nucleic acid comprises a sequence complementary to an RNA of an oncogene. In preferred embodiments, the oncogene includes, but is not limited to, abl, Bcl-2, Bcl-xL, erb, fms, gsp, hst, jun, myc, neu, raf; ras, ret, src, or trk. In some embodiments, the nucleic acid encoding a therapeutic protein encodes a factor including, but not limited to, a tumor suppressor, cytokine, receptor, inducer of apoptosis, or differentiating agent. In preferred embodiments, the tumor suppressor includes, but is not limited to, BRCA1, BRCA2, C-CAM, p16, p21, p53, p73, Rb, and p27. In preferred embodiments, the cytokine includes, but is not limited to, GMCSF, IL-1, IL-2, IL-3, IL-4, IL-5, IL-6, IL-7, IL-8, IL-9, IL-10, IL-11, IL-12, IL-13, IL-14, IL-15, β-interferon, γ-interferon, and TNF. In preferred embodiments, the receptor includes, but is not limited to, CFTR, EGFR, estrogen receptor, IL-2 receptor, and VEGFR. In preferred embodiments, the inducer of apoptosis includes, but is not limited to, AdElB, Bad, Bak, Bax, Bid, Bik, Bim, Harakid, and ICE-CED3 protease. In some embodiments, the therapeutic agent comprises a short-half life radioisotope.

In some embodiments of the present invention, the biological monitoring agent comprises an agent that measures an effect of a therapeutic agent (e.g., directly or indirectly measures a cellular factor or reaction induced by a therapeutic agent), however, the present invention is not limited by the nature of the biological monitoring agent. In some embodiments, the monitoring agent is capable of detecting (e.g., measuring) apoptosis caused by the therapeutic agent.

In some embodiments of the present invention, the imaging agent comprises a radioactive label including, but not limited to ¹⁴C, ³⁶Cl, ⁵⁷Co, ⁵⁸Co, ⁵¹Cr, ¹²⁵I, ¹³¹I, ¹¹¹Ln, ¹⁵²Eu, ⁵⁹Fe, ⁶⁷Ga, ³²P, ¹⁸⁶Re, ³⁵S, ⁷⁵Se, Tc-99m, and ¹⁷⁵Yb. In some embodiments, the imaging agent comprises a fluorescing entity. In a preferred embodiment, the imaging agent is fluorescein isothiocyanate or 6-TAMARA.

In some embodiments of the present invention, the targeting agent includes, but is not limited to an antibody, receptor ligand, hormone, vitamin, and antigen, however, the present invention is not limited by the nature of the targeting agent. In some embodiments, the antibody is specific for a disease-specific antigen. In some preferred embodiments, the disease-specific antigen comprises a tumor-specific antigen. In some embodiments, the receptor ligand includes, but is not limited to, a ligand for CFTR, EGFR, estrogen receptor, FGR2, folate receptor, IL-2 receptor, glycoprotein, and VEGFR. In a preferred embodiment, the receptor ligand is folic acid.

In some embodiments, the dendrimers of the present invention are configured to treat disease. In preferred embodiments, the dendrimers of the present invention are configured such that they are readily cleared from the subject (e.g., so that there is little to no detectable toxicity at efficacious doses). In some embodiments, the disease is a neoplastic disease, selected from, but not limited to, leukemia, acute leukemia, acute lymphocytic leukemia, acute myelocytic leukemia, myeloblastic, promyelocytic, myelomonocytic, monocytic, erythroleukemia, chronic leukemia, chronic myelocytic, (granulocytic) leukemia, chronic lymphocytic leukemia, Polycythemia vera, lymphoma, Hodgkin's disease, non-Hodgkin's disease, Multiple myeloma, Waldenstrom's macroglobulinemia, Heavy chain disease, solid tumors, sarcomas and carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, and neuroblastomaretinoblastoma. In some embodiments, the disease is an inflammatory disease selected from the group consisting of, but not limited to, eczema, inflammatory bowel disease, rheumatoid arthritis, asthma, psoriasis, ischemia/reperfusion injury, ulcerative colitis and acute respiratory distress syndrome. In some embodiments, the disease is a viral disease selected from the group consisting of, but not limited to, viral disease caused by hepatitis B, hepatitis C, rotavirus, human immunodeficiency virus type I (HIV-I), human immunodeficiency virus type II (HIV-II), human T-cell lymphotropic virus type I (HTLV-I), human T-cell lymphotropic virus type II (HTLV-II), AIDS, DNA viruses such as hepatitis type B and hepatitis type C virus; parvoviruses, such as adeno-associated virus and cytomegalovirus; papovaviruses such as papilloma virus, polyoma viruses, and SV40; adenoviruses; herpes viruses such as herpes simplex type I (HSV-I), herpes simplex type II (HSV-II), and Epstein-Barr virus; poxviruses, such as variola (smallpox) and vaccinia virus; and RNA viruses, such as human immunodeficiency virus type I (HIV-I), human immunodeficiency virus type II (HIV-II), human T-cell lymphotropic virus type I (HTLV-I), human T-cell lymphotropic virus type II (HTLV-II), influenza virus, measles virus, rabies virus, Sendai virus, picornaviruses such as poliomyelitis virus, coxsackieviruses, rhinoviruses, reoviruses, togaviruses such as rubella virus (German measles) and Semliki forest virus, arboviruses, and hepatitis type A virus.

### DESCRIPTION OF THE DRAWINGS

FIG. 1 depicts the (A) classical process, versus the (B) process in some embodiments of the present invention, used to synthesize PAPAM dendrimers.

FIG. 2 depicts a preferred protecting group (PG) of the protected core domain.

FIG. 3 depicts a core diamine when the core diamine is phenylenediamine, N—((CH₂)ₙ—NH₂)₃ (n=1-10).

FIG. 4 depicts the phenylenediamine of FIG. 3, but with substituents, where R and R1 are independently selected to be hydrogen, C1-C6 straight-chain or branched alkyls, C3-C6 cycloalkyls, C5-C10 aryl unsubstituted or substituted with C1-C6 alkyls, C1-C6 alkoxyls, 1,3-dioxolanyl, trihaloalkyl, carboxyl, C1-C6 dialkylamino, C1-C6 sulfanatoalkyl, C1-C6 sulfamylalkyl, or C1-C6 phosphanatoalkyl.

FIG. 5 depicts the synthesis of the phenylenediamines by catalytic reduction of the commercially available phenylenebisacetonitriles.

FIG. 6 depicts a synthetic scheme for generating G5 PAMAM dendrimers.

FIG. 7 depicts potentiometric titration curves of G5 PAMAM dendrimers.

FIG. 8 depicts gel permeation chromatography eluograms of the partially acetylated carrier and final products, with the RI signal and laser light scattering signal at 90° overlapping.

FIG. 9 depicts the theoretical and defected chemical structures of the G5 PAMAM dendrimer.

FIG. 10 depicts the (A) H1-NMR spectrum and (B) HPLC eluogram of the G5-Ac2 dendrimer.

FIG. 11 depicts the chemical structures of fluorescein isothiocyanate, folic acid and methotrexate, with the group used for conjugation marked with an asterisk.

FIG. 12 depicts the proton NMR imaging of fluorescein isothiocyanate, folic acid and methotrexate.

FIG. 13 depicts the HPLC eluogram of (A) G5-Ac²-FITC-OH-MTXe and (B) G5-Ac³-FITC-OH-MTX^{e} at 305 nm.

FIG. 14 depicts the H1-NMR spectrum of G5-Ac²-FITC-FA-OH-MTX^{e}.

FIG. 15 depicts the HPLC eluogram of G5-Ac-FITC-FA-OH-MTXe at 305 nm.

FIG. 16 depicts the UV spectra of free fluorescein isothiocyanate, folic acid and methotrexate.

FIG. 17 depicts the UV spectra of G5-Ac, G5-Ac³-FITC, G5-Ac³-FITC-FA, and G5-Ac³-FITC-FA-MTXe.

FIG. 18 depicts the (A) raw and (B) normalized fluorescence of dose-dependent binding of G5-FITC-FA-MTX in KB cells.

FIG. 19 depicts the effect of free FA on the uptake of the G5-FITC-FA and G5-FITC-FA-MTX in KB cells expressing high and low FA receptor.

FIG. 20 depicts confocal microscopy of KB cells treated with dendrimers.

FIG. 21 depicts (A) time course and (B) dose-dependent inhibition of cell growth using dendrimers.

FIG. 22 depicts growth inhibition of KB cells by dendrimers determined by XTT assays.

FIG. 23 depicts a comparison of cell growth inhibition using G5-FITC-FA-MTX and equimolar concentrations of mixtures of MTX and free FA.

FIG. 24 depicts reversal of G5-FA-MTX-induced inhibition of cell growth by free FA.

FIG. 25 depicts dendrimer stability in cell culture medium.

FIG. 26 depicts cytotoxicity of the dendrimers.

FIG. 27 shows the biodistribution of radiolabeled (A) nontargeted and (B) targeted conjugate in nu/nu mice bearing KB xenograft tumor depicted as a percentage of injected dose of dendrimer recovered per gram of organ.

FIG. 28 shows confocal microscopy analysis of cryosectioned tumor samples from SCID mice that were injected with 10 nmol of either (A) nontargeted G5-6-TAMRA or (B) targeted G5-FA-6-TAMRA conjugate (B) 15 hours or (D) 4 days before tumor isolation. Specific uptake by tumor cells of G5-FA-6-TAMRA versus G5-6-TAMRA is shown in (C).

FIG. 29 depicts tumor growth in SCID mice bearing KB xenografts during treatment with G5-FI-FA-MTX conjugate and free methotrexate (MTX).

FIG. 30 depicts survival rate of SCID mice bearing KB tumors.

FIG. 31 depicts a synthesis scheme for G5-Ac-AF-RGD.

FIG. 32 shows binding of G5-Ac-AF-RGD to HUVEC cells.

FIG. 33 shows binding of G5-Ac-AF-RGD to various cell lines.

FIG. 34 shows the dose dependent binding of G5-Ac-AF-RGD to HUVEC cells determined by confocal microscopy.

FIG. 35 shows the inhibition of uptake of G5-Ac-AF-RGD by HUVEC cells with addition of free peptide.

FIG. 36 depicts a synthesis scheme for a G5-PMPA dendrimer.

FIG. 37 shows a schematic representation of the fabrication of targeted iron oxide nanoparticles (Fe₃O₄ NPs).

FIG. 38 shows transmission electron microscope (TEM) images of PSS/G5.NHAc-FI-FA-coated Fe₃O₄ NPs (a) without and (b) with phosphotungstic acid negative staining.

FIG. 39 shows cytometric analysis of binding of Fe₃O₄ NPs with KB cells: Binding of Fe₃O₄ NPs modified with PSS/G5.NHAc-FI and PSS/G5.NHAc-FI-FA bilayers with KB cells expressing (a) high and (b) low level folic acid receptor (FAR) 1. PBS control; 2. PSS/G5.NHAc-FI-modified Fe₃O₄ NPs; 3. PSS/G5.NHAc-FI-FA-modified Fe₃O₄ NPs; and dose-dependent binding of functionalized Fe₃O₄ NPs with KB cells expressing (c) high and (d) low level FAR.

FIG. 40 shows confocal microscopic imaging of KB cells with high-level FAR treated with (a) PBS buffer, (b) Fe₃O₄/PSS/G5.NHAc-FI, or (c) Fe₃O₄/PSS/G5.NHAc-FI-FA for 2 h, respectively.

FIG. 41 shows transmission electron microscope (TEM) micrographs of KB-HFAR cells treated with Fe₃O₄/PSS/G5.NHAc-FI-FA (a and b) and Fe₃O₄/PSS/G5.NHAc-FI (c) for 2 h. (b) shows a magnified area of a typical vacuolar structure of a KB-HFAR cell.

FIG. 42 shows magnetic resonance (MR) imaging of KB-HFAR cell pellets incubated with functionalized Fe₃O₄ NPs. (a) T2 weighted spin-echo images of cells incubated with functionalized Fe₃O₄ NPs with Fe concentrations of 0, 22.5, 45, 90 μg/mL. (b) The percentage of signal intensity compared to the cells in PBS was plotted to the Fe concentration for both Fe₃O₄/PSS/G5.NHAc-FI-FA and Fe₃O₄/PSS/G5.NHAc-FI NPs.

FIG. 43 shows (a) a TEM micrograph and (b) the size distribution histogram of pristine Fe₃O₄ nanocrystals synthesized by controlled co-precipitation of Fe(II) and Fe(III) ions.

FIG. 44 shows an MTT assay of KB cell viability after treatment with Fe₃O₄/PSS/G5.NHAc-FI (non-targeted) and Fe₃O₄/PSS/G5.NHAc-FI-FA (targeted) NPs with Fe concentration of 45 μg/mL for 24 h.

FIG. 45 shows phase-contrast photomicrographs of control KB cells (a) without treatment, (b) KB cells treated with Fe₃O₄/PSS/G5.NHAc-FI-FA NPs with Fe concentrations of 225 μg/mL, (c) KB cells treated with Fe₃O₄/PSS/G5.NHAc-FI-FA NPs with Fe concentrations of 360 μg/mL, (d) KB cells treated with Fe₃O₄/PSS/G5.NHAc-FI NPs with Fe concentrations of 225 μg/mL, and (e) KB cells treated with Fe₃O₄/PSS/G5.NHAc-FI NPs with Fe concentrations of 360 μg/mL for 96 h.

FIG. 46 shows a TEM image of the minimal uptake of Fe₃O₄/PSS/G5.NHAc-FI NPs in the vacuoles of KB-HFAR cells.

FIG. 47 depicts a schematic representation of the procedure for fabricating multifunctional shell-crosslinked iron oxide (SCIO) nanoparticles (NPs).

FIG. 48 shows the zeta potential data of iron oxide NPs after each step of assembly, crosslinking, and chemical modification.

FIG. 49 shows TEM characterization. (a) TEM image of SCIO-FA NPs; (b) A negatively phosphotungstic acid-stained TEM images of SCIO-FA NPs. TEM images of (PGA/PLL)₂/PGA/G5.NH₂-FI-FA hollow polymer capsules after iron oxide core removal are also shown. (c) (PGA/PLL)₂/PGA/G5.NH₂-FI-FA intact hollow polymer capsules before EDC crosslinking; (d) A magnified image of the capsule indicated by a vertical arrow; (e) A magnified image of the capsule indicated by a horizontal arrow; (f) (PGA/PLL)₂/PGA/G5.NH₂-FI-FA broken hollow polymer capsules after EDC crosslinking.

FIG. 50 shows FTIR spectra of (PGA/PLL)₂/PGA/G5.NH₂-FI-FA-modified Fe₃O₄ NPs before and after EDC crosslinking.

FIG. 51 shows linear fitting of inverse T₂ relaxation times of uncoated Fe₃O₄ NPs, SCIO-FA NPs, and SCIO-NonFA NPs.

FIG. 52 shows KB cell viability measured by staining with FDA and PI after incubation with the NPs for 24 h. Fluorescence was analyzed by flow cytometry. Ten thousand cells per sample were acquired for analysis. The percentage of FDA positive stained cells after treatment of unmodified Fe₃O₄ NPs, SCIO-NonFA NPs, and SCIO-FA NPs were compared.

FIG. 53 shows in vitro flow cytometric analysis of binding of SCIO NPs with KB cells and MRI of cell pellets. Dose-dependent binding of SCIO-FA and SCIO-NonFA NPs with KB cells expressing high—(a) and low—(b) level FAR. T₂ weighted spin-echo images of KB-HFAR cells incubated with functionalized SCIO-FA and SCIO-NonFA NPs with Fe concentrations of 0, 6.3, 12.5, 25.0 μg/mL (c).

FIG. 54 shows the T₂ of KB-HFAR cells treated with functionalized Fe₃O₄ NPs.

FIG. 55 shows in vivo MRI of tumor. In vivo color maps (a) of T₂-weighted MR images of a mouse implanted with cancer cell line KB cells, at different time points after injection of SCIO-NonFA and SCIO-FA NPs, respectively. The color bar (from red to blue) indicates the MRI signal intensity changes from high to low. Comparison of statistically normalized histograms of the voxel intensities (whole tumor) from targeted SCIO-FA (green histogram) and non-targeted SCIO-NonFA (red histogram) NPs at the time points of 1 h (b), 4 h (c), 8 h (d), 24 h (e), 48 h (f), and 7 days (g).

FIG. 56 shows the biodistribution of SCIO NPs. MRI signal intensity of tumor, liver, kidney, and muscle for control mice, mice treated with SCIO-FA NPs, and mice treated with SCIO-NonFA NPs.

### DEFINITIONS

To facilitate an understanding of the present invention, a number of terms and phrases are defined below:

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the terms “epithelial tissue” or “epithelium” refer to the cellular covering of internal and external surfaces of the body, including the lining of vessels and other small cavities. It consists of cells joined by small amounts of cementing substances. Epithelium is classified into types on the basis of the number of layers deep and the shape of the superficial cells.

As used herein, the term “normal epithelium of prostate” refers to prostate epithelium that does not show any detectable indication of cancerous or pre-cancerous conditions.

As used herein, the term “cancerous epithelium of prostate” refers to prostate epithelium that shows a detectable indication of cancerous or pre-cancerous conditions.

As used herein, the term “subject suspected of having cancer” refers to a subject that presents one or more symptoms indicative of a cancer (e.g., a noticeable lump or mass) or is being screened for a cancer (e.g., during a routine physical). A subject suspected of having cancer may also have one or more risk factors. A subject suspected of having cancer has generally not been tested for cancer. However, a “subject suspected of having cancer” encompasses an individual who has received a preliminary diagnosis (e.g., a CT scan showing a mass) but for whom a confirmatory test (e.g., biopsy and/or histology) has not been done or for whom the stage of cancer is not known. The term further includes people who once had cancer (e.g., an individual in remission). A “subject suspected of having cancer” is sometimes diagnosed with cancer and is sometimes found to not have cancer.

As used herein, the terms “prostate specific membrane antigent” or “PSMA” refer to a membrane-bound epitope, originally identified by Horoszewicz et al. (See, e.g., Horoszewicz et al., Anticancer Res 7, 927, (1987); van Steenbrugge et al., Urol Res 17, 71 (1989); Carter et al., Proc Natl Acad Sci USA. 93(2): 749 (1996)), selectively expressed in epithelial cells of prostatic origin. Small amounts of PSMA expression have been detected in a variety of tumors (See, e.g., Chang et al., Clin Cancer Res 5, 2674 (1999)).

As used herein, the term “subject diagnosed with a cancer” refers to a subject who has been tested and found to have cancerous cells. The cancer may be diagnosed using any suitable method, including but not limited to, biopsy, x-ray, blood test, and the diagnostic methods of the present invention. A “preliminary diagnosis” is one based only on visual (e.g., CT scan or the presence of a lump) and antigen tests (e.g., PSMA).

As used herein, the term “initial diagnosis” refers to a test result of initial cancer diagnosis that reveals the presence or absence of cancerous cells (e.g., using a biopsy and histology).

As used herein, the term “prostate tumor tissue” refers to cancerous tissue of the prostate. In some embodiments, the prostate tumor tissue is “post surgical prostate tumor tissue.”

As used herein, the term “post surgical tumor tissue” refers to cancerous tissue (e.g., prostate tissue) that has been removed from a subject (e.g., during surgery).

As used herein, the term “identifying the risk of said tumor metastasizing” refers to the relative risk (e.g., the percent chance or a relative score) of a tumor (e.g., prostate tumor tissue) metastasizing.

As used herein, the term “identifying the risk of said tumor recurring” refers to the relative risk (e.g., the percent chance or a relative score) of a tumor (e.g., prostate tumor tissue) recurring in the same organ as the original tumor (e.g., prostate).

As used herein, the term “subject at risk for cancer” refers to a subject with one or more risk factors for developing a specific cancer. Risk factors include, but are not limited to, gender, age, genetic predisposition, environmental expose, and previous incidents of cancer, preexisting non-cancer diseases, and lifestyle.

As used herein, the term “characterizing cancer in subject” refers to the identification of one or more properties of a cancer sample in a subject, including but not limited to, the presence of benign, pre-cancerous or cancerous tissue and the stage of the cancer. Cancers may be characterized by identifying cancer cells with the compositions and methods of the present invention. For example, cancers may be characterized by detecting expression of PSMA with the compositions and methods of the present invention.

As used herein, the term “stage of cancer” refers to a qualitative or quantitative assessment of the level of advancement of a cancer. Criteria used to determine the stage of a cancer include, but are not limited to, the size of the tumor, whether the tumor has spread to other parts of the body and where the cancer has spread (e.g., within the same organ or region of the body or to another organ).

Several staging methods are commonly used for cancer (e.g., prostate cancer). A common classification of the spread of prostate cancer was developed by the American Urological Association (AUA). The AUA system divides prostate tumors into four stages, A to D. Stage A, microscopic cancer within prostate, is further subdivided into stages A1 and A2. Sub-stage A1 is a well-differentiated cancer confined to one site within the prostate. Treatment is generally observation, radical prostatectomy, or radiation. Sub-stage A2 is a moderately to poorly differentiated cancer at multiple sites within the prostate. Treatment is radical prostatectomy or radiation. Stage B, palpable lump within the prostate, is also further subdivided into sub-stages B1 and B2. In sub-stage B1, the cancer forms a small nodule in one lobe of the prostate. In sub-stage B2, the cancer forms large or multiple nodules, or occurs in both lobes of the prostate. Treatment for sub-stages B1 and B2 is either radical prostatectomy or radiation. Stage C is a large cancer mass involving most or all of the prostate and is also further subdivided into two sub-stages. In sub-stage C₁, the cancer forms a continuous mass that may have extended beyond the prostate. In sub-stage C2, the cancer forms a continuous mass that invades the surrounding tissue. Treatment for both these sub-stages is radiation with or without drugs to address the cancer. The fourth stage, Stage D is metastatic cancer and is also subdivided into two sub-stages. In sub-stage D1, the cancer appears in the lymph nodes of the pelvis. In sub-stage D2, the cancer involves tissues beyond lymph nodes. Treatment for both of these sub-stages is systemic drugs to address the cancer as well as pain.

As used herein, the term “GLEASON score” refers to a histologic grade that refers to the microscopic characteristics of malignant prostatic tumor. Individual areas receive a grade from 1 to 5. Cells that are well differentiated are given a low grade; poorly differentiated cells are given a high grade. A primary grade is assigned to the pattern occupying the greatest area of the specimen and a secondary grade is assigned to the second-largest affected area. These two grades are then added together for an overall Gleason score (or sum). The most well-differentiated cancer would receive a Gleason score of 2 (1+1), while the most poorly differentiated cancer would receive a Gleason score of 10 (5+5).

Staging of prostate cancer can also be based on the revised criteria of TNM staging by the American Joint Committee for Cancer (AJCC) published in 1988. Staging is the process of describing the extent to which cancer has spread from the site of its origin. It is used to assess a patient's prognosis and to determine the choice of therapy. The stage of a cancer is determined by the size and location in the body of the primary tumor, and whether it has spread to other areas of the body. Staging involves using the letters T, N and M to assess tumors by the size of the primary tumor (T); the degree to which regional lymph nodes (N) are involved; and the absence or presence of distant metastases (M)—cancer that has spread from the original (primary) tumor to distant organs or distant lymph nodes. Each of these categories is further classified with a number 1 through 4 to give the total stage. Once the T, N and M are determined, a “stage” of I, II, III or IV is assigned. Stage I cancers are small, localized and usually curable. Stage II and III cancers typically are locally advanced and/or have spread to local lymph nodes. Stage 1V cancers usually are metastatic (have spread to distant parts of the body) and generally are considered inoperable.

As used herein, the term “characterizing tissue in a subject” refers to the identification of one or more properties of a tissue sample (e.g., including but not limited to, the presence of cancerous tissue, the presence of pre-cancerous tissue that is likely to become cancerous, and the presence of cancerous tissue that is likely to metastasize). In some embodiments, tissues are characterized detecting expression of PSMA with the compositions and methods of the present invention.

As used herein, the term “reagent(s) capable of specifically detecting PSMA expression” refers to reagents used to detect the expression and location of PSMA. Examples of suitable reagents include but are not limited to, the dendrimers of the present invention

As used herein, the term “instructions for using said kit for detecting cancer in said subject” includes instructions for using the reagents contained in the kit for the detection and characterization of cancer in a sample from a subject.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the terms “processor” and “central processing unit” or “CPU” are used interchangeably and refer to a device that is able to read a program from a computer memory (e.g., ROM or other computer memory) and perform a set of steps according to the program.

As used herein, the term “providing a prognosis” refers to providing information regarding the impact of the presence of cancer (e.g., as determined by the diagnostic methods of the present invention) on a subject's future health (e.g., expected morbidity or mortality, the likelihood of getting cancer, and the risk of metastasis).

As used herein, the term “non-human animals” refers to all non-human animals including, but not limited to, vertebrates such as rodents, non-human primates, ovines, bovines, ruminants, lagomorphs, porcines, caprines, equines, canines, felines, aves, etc.

As used herein, the term “gene transfer system” refers to any means of delivering a composition comprising a nucleic acid sequence to a cell or tissue. For example, gene transfer systems include, but are not limited to, vectors (e.g., retroviral, adenoviral, adeno-associated viral, and other nucleic acid-based delivery systems), microinjection of naked nucleic acid, dendrimers, polymer-based delivery systems (e.g., liposome-based and metallic particle-based systems), biolistic injection, and the like. As used herein, the term “viral gene transfer system” refers to gene transfer systems comprising viral elements (e.g., intact viruses, modified viruses and viral components such as nucleic acids or proteins) to facilitate delivery of the sample to a desired cell or tissue. As used herein, the term “adenovirus gene transfer system” refers to gene transfer systems comprising intact or altered viruses belonging to the family Adenoviridae.

As used herein, the term “site-specific recombination target sequences” refers to nucleic acid sequences that provide recognition sequences for recombination factors and the location where recombination takes place.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N-6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N-6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “transgene” refers to a heterologous gene that is integrated into the genome of an organism (e.g., a non-human animal) and that is transmitted to progeny of the organism during sexual reproduction.

As used herein, the term “transgenic organism” refers to an organism (e.g., a non-human animal) that has a transgene integrated into its genome and that transmits the transgene to its progeny during sexual reproduction.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the terms “an oligonucleotide having a nucleotide sequence encoding a gene” and “polynucleotide having a nucleotide sequence encoding a gene,” means a nucleic acid sequence comprising the coding region of a gene or in other words the nucleic acid sequence that encodes a gene product. The coding region may be present in a cDNA, genomic DNA or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, for the sequence “5′-A-G-T-3′,” is complementary to the sequence “5′-T-C-A-3′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

When used in reference to a single-stranded nucleic acid sequence, the term “substantially homologous” refers to any probe that can hybridize (i.e., it is the complement of) the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.” As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization (1985)). Other references include more sophisticated computations that take structural as well as sequence characteristics into account for the calculation of Tₘ.

“Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

Template specificity is achieved in most amplification techniques by the choice of enzyme. Amplification enzymes are enzymes that, under conditions they are used, will process only specific sequences of nucleic acid in a heterogeneous mixture of nucleic acid. For example, in the case of Qβ replicase, MDV-1 RNA is the specific template for the replicase (Kacian et al., Proc. Natl. Acad. Sci. USA 69:3038 (1972)). Other nucleic acids will not be replicated by this amplification enzyme. Similarly, in the case of T7 RNA polymerase, this amplification enzyme has a stringent specificity for its own promoters (Chamberlin et al., Nature 228:227 (1970)). In the case of T4 DNA ligase, the enzyme will not ligate the two oligonucleotides or polynucleotides, where there is a mismatch between the oligonucleotide or polynucleotide substrate and the template at the ligation junction (Wu and Wallace, Genomics 4:560 (1989)). Finally, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.), PCR Technology, Stockton Press (1989)).

As used herein, the term “amplifiable nucleic acid” is used in reference to nucleic acids that may be amplified by any amplification method. It is contemplated that “amplifiable nucleic acid” will usually comprise “sample template.”

As used herein, the term “sample template” refers to nucleic acid originating from a sample that is analyzed for the presence of “target.” In contrast, “background template” is used in reference to nucleic acid other than sample template that may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the term “target,” refers to the region of nucleic acid bounded by the primers. Thus, the “target” is sought to be sorted out from other nucleic acid sequences. A “segment” is defined as a region of nucleic acid within the target sequence.

As used herein, the term “amplification reagents” refers to those reagents (deoxyribonucleotide triphosphates, buffer, etc.), needed for amplification except for primers, nucleic acid template and the amplification enzyme. Typically, amplification reagents along with other reaction components are placed and contained in a reaction vessel (test tube, microwell, etc.).

As used herein, the terms “restriction endonucleases” and “restriction enzymes” refer to bacterial enzymes, each of which cut double-stranded DNA at or near a specific nucleotide sequence.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

“Amino acid sequence” and terms such as “polypeptide” or “protein” are not meant to limit the amino acid sequence to the complete, native amino acid sequence associated with the recited protein molecule.

The term “native protein” as used herein to indicate that a protein does not contain amino acid residues encoded by vector sequences; that is, the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four amino acid residues to the entire amino acid sequence minus one amino acid.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al., Molecular Cloning: A Laboratory Manual, Cold Spring Harbor Press, NY, pp 9.31-9.58 (1989)).

The term “Northern blot,” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (J. Sambrook, et al., supra, pp 7.39-7.52 (1989)).

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabeled antibodies.

As used herein, the term “vector” is used in reference to nucleic acid molecules that transfer DNA segment(s) from one cell to another. The term “vehicle” is sometimes used interchangeably with “vector.” Vectors are often derived from plasmids, bacteriophages, or plant or animal viruses.

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The terms “overexpression” and “overexpressing” and grammatical equivalents, are used in reference to levels of mRNA to indicate a level of expression approximately 3-fold higher (or greater) than that observed in a given tissue in a control or non-transgenic animal. Levels of mRNA are measured using any of a number of techniques known to those skilled in the art including, but not limited to Northern blot analysis. Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the mRNA-specific signal observed on Northern blots). The amount of mRNA present in the band corresponding in size to the correctly spliced transgene RNA is quantified; other minor species of RNA which hybridize to the transgene probe are not considered in the quantification of the expression of the transgenic mRNA.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “calcium phosphate co-precipitation” refers to a technique for the introduction of nucleic acids into a cell. The uptake of nucleic acids by cells is enhanced when the nucleic acid is presented as a calcium phosphate-nucleic acid co-precipitate. The original technique of Graham and van der Eb (Graham and van der Eb, Virol., 52:456 (1973)), has been modified by several groups to optimize conditions for particular types of cells. The art is well aware of these numerous modifications.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

As used herein, the term “selectable marker” refers to the use of a gene that encodes an enzymatic activity that confers the ability to grow in medium lacking what would otherwise be an essential nutrient (e.g. the HIS3 gene in yeast cells); in addition, a selectable marker may confer resistance to an antibiotic or drug upon the cell in which the selectable marker is expressed. Selectable markers may be “dominant”; a dominant selectable marker encodes an enzymatic activity that can be detected in any eukaryotic cell line. Examples of dominant selectable markers include the bacterial aminoglycoside 3′ phosphotransferase gene (also referred to as the neo gene) that confers resistance to the drug G418 in mammalian cells, the bacterial hygromycin G phosphotransferase (hyg) gene that confers resistance to the antibiotic hygromycin and the bacterial xanthine-guanine phosphoribosyl transferase gene (also referred to as the gpt gene) that confers the ability to grow in the presence of mycophenolic acid. Other selectable markers are not dominant in that their use must be in conjunction with a cell line that lacks the relevant enzyme activity. Examples of non-dominant selectable markers include the thymidine kinase (tk) gene that is used in conjunction with tk⁻ cell lines, the CAD gene that is used in conjunction with CAD-deficient cells and the mammalian hypoxanthine-guanine phosphoribosyl transferase (hprt) gene that is used in conjunction with hprt⁻ cell lines. A review of the use of selectable markers in mammalian cell lines is provided in Sambrook, J. et al., Molecular Cloning: A Laboratory Manual, 2nd ed., Cold Spring Harbor Laboratory Press, New York (1989) pp. 16.9-16.15.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used herein, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., cancer). Test compounds comprise both known and potential therapeutic compounds. A test compound can be determined to be therapeutic by screening using the screening methods of the present invention.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

As used herein, the term “NAALADase inhibitor” refers to any one of a multitude of inhibitors for the neuropeptidase NAALADase (N-acetylated-alpha linked acidic dipeptidase). Such inhibitors of NAALADase have been well characterized. For example, an inhibitor can be selected from the group comprising, but not limited to, those found in U.S. Pat. No. 6,011,021, herein incorporated by reference in its entirety.

As used herein, the term “nanodevice” or “nanodevices” refer, generally, to compositions comprising dendrimers of the present invention. As such, a nanodevice may refer to a composition comprising a dendrimer and metal nanoparticles (e.g., iron oxide nanoparticles (e.g., poly(styrene sulfonate) (PSS)-coated iron oxide nanoparticles)) of the present invention that may contain one or more functional groups (e.g., a therapeutic agent) conjugated to the dendrimer. A nanodevice may also refer to a composition comprising two or more different dendrimers of the present invention.

### DETAILED DESCRIPTION OF THE INVENTION

The present invention provides novel systems and compositions for the treatment and monitoring (e.g., imaging) of diseases (e.g., cancer). For example, the present invention provides systems and compositions that target, image, and/or sense pathophysiological defects, provide the appropriate therapeutic based on the diseased state, monitor the response to the delivered therapeutic, and/or identify residual disease. In preferred embodiments, the compositions of the present invention are small enough to readily enter a patient's or subjects cells and to be cleared from the body with little to no toxicity at therapeutic or functional (e.g., imaging) doses.

Accordingly, in some embodiments, the present invention provides methods of synthesizing dendrimer conjugates (e.g., PAMAM dendrimers) comprising metal nanoparticles (NPs) (e.g., iron oxide (Fe₃O₄) NPs), compositions comprising the same, and methods of using the same in the diagnosis, imaging and treatment of cancer (e.g., prostate cancer).

In some embodiments, the present invention provides iron oxide NPs conjugated to a dendrimer and methods of synthesizing the same (See, e.g., Examples 18 and 19). In some embodiments, a dendrimer comprising iron oxide NPs comprises one or more functional groups selected from the group comprising, but not limited to, a therapeutic agent, an imaging agent, a targeting agent, and a biological monitoring agent. In some embodiments, dendrimers comprising iron oxide NPs are utilized for imaging cancerous tissue (e.g., a tumor or metastasis). In some embodiments, dendrimers of the present invention (e.g., dendrimers comprising iron oxide NPs and one or more functional groups) target cells (e.g., tumor cells possessing a targeting moiety (e.g., a folic acid receptor)) for delivery of one or more functional groups (e.g., therapeutic agent, imaging agent, or biological monitoring agent) to the cell.

In some embodiments, a layer-by-layer (LbL) self-assembly method is utilized in combination with dendrimer synthesis chemistry in order to generate dendrimers comprising iron oxide NPs of the present invention (e.g., dendrimers comprising functionalized Fe₃O₄ nanoparticles (NPs) that further comprise folic acid (FA) and fluorescein isothiocyanate (FI) (e.g., for targeting, imaging and/or treatment of cancer cells) (See, e.g., Examples 18 and 19)).

Fe₃O₄ NPs generated via processes that combine a layer-by-layer (LbL) self-assembly techniques and dendrimer chemistry can specifically target to tumors cells overexpressing folic acid receptor (FAR) in vitro (See, e.g., Example 18). For example, a bilayer composed of polystyrene sulfonate sodium salt (PSS) and FA- and FI (fluorescein isothiocyanate)-functionalized poly(amidoamine) (PAMAM) dendrimers of generation 5 (G5.NH₂-FI-FA) can be assembled onto Fe₃O₄ NPs through electrostatic LbL assembly, followed by acetylation of the remaining surface amine groups of the assembled G5 dendrimers, and are able to target tumor cells expressing FAR in vitro. Unfortunately, in vivo data generated using these NPs demonstrated that most of the bilayer-modified Fe₃O₄ NPs accumulate in the liver of mice, suggesting that the particles lack in vivo stability. Although an understanding of the mechanism is not necessary to practice the present invention and the present invention is not limited to any particular mechanism of action, in some embodiments, the bilayer-modified Fe₃O₄ NPs lack in vivo stability due to components of and/or the thickness of the polymer shell, weak mechanical stability, and/or the polymer not being biocompatible. Thus, experiments were conducted to determine whether multifunctional dendrimers (e.g., comprising targeting, imaging and/or therapeutic moieties) could be generated with iron oxide NPs in such a way so as to be biologically useful (e.g., for successful in vivo MRI of a tumor (See, e.g., Example 19)).

For example, in some embodiments, the present invention provides metal (e.g., iron oxide) NPs assembled with macromolecule (e.g., polymer) pairs (e.g., biocompatible macromolecule pairs) comprising a first polymer (e.g., positively charged polymer) comprising free amino groups and a second polymer (e.g., a negatively charged polymer) comprising free carboxyl groups (See, e.g., Example 19). The present invention is not limited by the type of macromolecule polymer pairs utilized. Indeed, a variety of macromolecule polymer pairs are contemplated to be useful in the present invention including, but not limited to, hyaluronic acid and poly-arginine; alginate and chitosan; poly-lactic acid and polylysine; etc. In some embodiments, the macromolecule polymer pair is poly(glutamic acid) (PGA) and poly-L-Lysine (PLL). Biocompatible polymers appear important as assembly of NPs with non-biocompatible polymers (non-biopolymers (e.g., polyacrylic acid and polyallyamine hydrochloride pairs)) display instability in vivo (e.g., multilayers of polyacrylic acid and polyallyamine hydrochloride on iron oxide NPs even after shell crosslinking are unstable in vivo and predominantly end up in the liver).

Accordingly, in some embodiments, the present invention provides synthesis methods that combine layer-by-layer (LbL) self-assembly methods with dendrimer chemistry for fabricating shell-crosslinked iron oxide (SCIO) nanoparticles (NPs) (e.g., that can be specifically targeted (e.g., to cells (e.g., for MRI of tumor cells and/or tissue))) (See, e.g., Example 19). Fabricated SCIO NPs are water-soluble, stable, and biocompatible. Both in vitro and in vivo MRI studies show that the SCIO NPs modified with a targeting moiety (e.g., folic acid (FA) modification (SCIO-FA NPs)) can specifically target to tumor cells (e.g., cells overexpressing FA receptor (FAR) or FAR-expressing tumors (e.g., tumors with a volume as small as 0.60±0.15 cm³) (See Example 19). The present invention is not limited to functionalizing metal nanoparticles. Indeed, shell crosslinking methods and compositions of the present invention may be applied to other small targeting molecules (e.g., peptides, growth factors, etc. (e.g., thereby providing an approach for MRI detection of various biological systems)).

In some embodiments, the present invention provides SCIO NPs assembled with poly(glutamic acid) (PGA) and poly-L-lysine (PLL), followed by assembly with dendrimers (e.g., G5.NH₂-FI-FA dendrimers (See, e.g., Example 19)). In some embodiments, the interlayers are crosslinked using 1-Ethyl-3-[3-dimethylaminopropyl]carbodiimide hydrochloride (EDC) chemistry to covalently link the hydroxyl groups of iron oxide, the carboxyl groups of PGA, and the amino groups of PLL and the dendrimers. The present invention is not limited to crosslinking with EDC chemistry. Indeed, a variety of methods can be used to crosslink NPs assembled with dendrimers (e.g., a dendrimer described herein (e.g., G5.NH₂-FI-FA)), including, but not limited to, Click chemistry, glutaraldehyde crosslinking, physical crosslinking (e.g., thermal crosslinking (e.g., to form amide bonds)), and/or UV irradiation crosslinking (e.g., of a carboxyl polymer assembled with a polycationic nitro-containing diazoresin). In some embodiments, the SCIO NPs (e.g., comprising dendrimers) undergo a surface neutralization reaction. For example, in some embodiments, free amino groups (e.g., remaining on the dendrimers post assembly and/or cross-linking) are acetylated (e.g., to neutralize surface charge). The present invention is not limited by the type of surface neutralization reaction utilized. In some embodiments, the surface neutralization reaction comprises an acetylation reaction. In some embodiments, the surface neutralization reactions comprises conjugating SCIO NPs (e.g., comprising dendrimers) to a water soluble polymer. The present invention is not limited by the type of water soluble polymer to which SCIO NPs are conjugated. Indeed, a variety of water soluble polymers may be utilized including, but not limited to, poly(alkylene oxides), polyoxyethylated polyols and poly(vinyl alcohols). Poly(alkylene oxides) include, but are not limited to, polyethylene glycols (PEGs), poloxamers and poloxamines. The present invention is not limited by the type of conjugation utilized (e.g., to connect SCIO NPs to one or more water-soluble polymers (e.g. PEG)). In some embodiments, a poly(alkylene oxide) is conjugated to a free amino group via an amide linkage (e.g., formed from an active ester (e.g., the N-hydroxysuccinimide ester)) of the poly(alkylene oxide). In some embodiments, an ester linkage remains in the conjugate after conjugation. In some embodiments, conjugation occurs through a short-acting, degradable linkage. The present invention is not limited by the type of degradable linkage utilized. Indeed, a variety of linkages are contemplated to be useful in the present invention including, but not limited to, physiologically cleavable linkages including ester, carbonate ester, carbamate, sulfate, phosphate, acyloxyalkyl ether, acetal, and ketal linkages. In some embodiments, SCIO NPs are conjugated to PEG utilizing any of the methods, reagents and/or linkages described in U.S. Pat. Nos. 4,424,311; 5,672,662; 6,515,100; 6,664,331; 6,737,505; 6,894,025; 6,864,350; 6,864,327; 6,610,281; 6,541,543; 6,515,100; 6,448,369; 6,437,025; 6,432,397; 6,362,276; 6,362,254; 6,348,558; 6,214,966; 5,990,237; 5,932,462; 5,900,461; 5,739,208; 5,446,090 and 6,828,401; and WO 02/02630 and WO 03/031581, and U.S. Pat. App. No. 60/786,188, each of which is herein incorporated by reference in its entirety. In some embodiments, the conjugate comprises a cleavable linkage present in the linkage between the polymer and SCIO NPs (e.g., such that when cleaved, no portion of the polymer or linkage remains on the SCIO NPs). In some embodiments, the conjugate comprises a cleavable linkage present in the polymer itself (e.g., such that when cleaved, a small portion of the polymer or linkage remains on the SCIO NPs.

The present invention also provides methods of characterizing shell-crosslinked iron oxide (SCIO) NPs including, but not limited to, characterizing using the zeta potential, Fourier transform infrared (FTIR) spectroscopy, transmission electron microscopy (TEM), and relativity measurements.

Electrostatic layer-by-layer self-assembly techniques have been described (See, e.g., Decher, Science 277, 1232-1237 (1997); Caruso et al., Science 282, 1111-1114 (1998)) that allows the creation of ultra-thin functional films (See, e.g., Schneider and Decher, Nano Lett. 4, 1833-1839 (2004); Schneider et al., Nano Lett. 6, 530-536 (2006); Gittins and Caruso, Adv. Mater. 12, 1947 (2000); Gittins and Caruso, J. Phys. Chem. B 105, 6846-6852 (2001); Thunemann et al., Langmuir 22, 2351-2357 (2006). In some embodiments, the biofunctionality of the films may be altered by deposition of functional polyelectrolytes or biomacromolecules on film surfaces (See, e.g., Wang et al., Nano Lett. 2, 857-861 (2002); Kato and Caruso, J. Phys. Chem. B 109, 19604-19612 (2005).

In some embodiments, synthesis of dendrimers of the present invention utilizes positively charged iron oxide NPs (e.g., about 8.4 nm in diameter, although, as described herein, smaller or larger NPs may be utilized) synthesized by controlled co-precipitation of Fe(II) and Fe(III) ions modified with a bilayer composed of polystyrene sulfonate sodium salt (PSS) and FA- and FI-functionalized poly(amidoamine) (PAMAM) dendrimers of generation 5 (G5.NH₂-FI-FA) through electrostatic LbL assembly. The present invention is not limited by the size of the iron oxide NPs associated with (e.g., conjugated to) a dendrimer of the present invention. In some embodiments, the iron oxide NPs are smaller than 8.4 nm in diameter (e.g., less than 8 nm, less than 7 nm, less than 6 nm, less than 5 nm, less than 4 nm, less than 3 nm, less than 2 nm, or smaller) or larger than 8.4 nm in diameter (e.g., greater than 9 nm, greater than 10 nm, greater than 12.5 nm, greater than 15 nm, or larger). In some embodiments, iron oxid NPs (e.g., SCIO NPs) assembled with dendrimers of the present invention are biocompatible at iron concentrations at about 0-10 μg/ml, at about 10-50 μg/ml, at about 50-75 μg/ml, at about 75-100 μg/ml (See, e.g., Example 19) or at high iron concentrations.

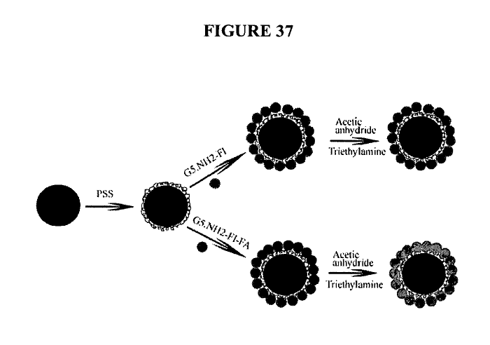

In some embodiments, bilayer-modified iron oxide NPs associated with (e.g., conjugated to) a dendrimer of the present invention are subjected to an acetylation reaction to neutralize remaining (e.g., non-reacted) terminal amine groups of G5 dendrimers (See, e.g., FIG. 37). Functionality of the dendrimers of the present invention can be characterized using a number of techniques including, but not limited to, flow cytometry, confocal microscopy, transmission electron microscopy (TEM), and magnetic resonance (MR) imaging studies. In preferred embodiments, a dendrimer of the present invention (e.g., Fe₃O₄/PSS/G5.NHAc-FI-FA NPs) specifically binds cancer cells (e.g., expressing a targeting moiety (e.g., FA receptor), See, e.g., Example 18). Dendrimers of the present invention find use in various settings and applications including, but not limited to, biological sensing and disease treatment (e.g., prophylactic and/or therapeutic) applications. For example, functionalizing iron oxide NPs using LbL self-assembly and dendrimer chemistries provides the ability to utilize various NPs and targeting ligands (e.g., sugars, peptides, antibodies or antibody fragments, hormones and the like), thereby providing the ability to generate specific strategies to fabricate various NPs for a range of biological sensing and therapeutics applications (e.g., to treat and/or prevent cancer).

In some embodiments, altering (e.g., increasing or decreasing) the polymer layer thickness of the iron oxide nanoparticles is utilized to increase or decrease stability (e.g., of the nanoparticles and/or the dendrimers associated with (e.g., conjugated to) the nanoparticles). For example, in some embodiments, increasing the polymer layer thickness increases the stability of the iron oxide nanoparticles (e.g., thereby stabilizing dendrimers associated with (e.g., conjugated to) the same). In some embodiments, the polymer layer thickness is altered by increasing the number of polymer layers through a versatile LbL self-assembly technique (e.g., described in Example 18). In some embodiments, the mechanical stability of the polymer coating is altered (e.g., improved) through shell cross-linking.

In some embodiments, detection of a targeting moiety (e.g. levels and location of FA receptor expression), as determined using compositions and methods of the present invention, are correlated with cancer stage and/or tumor volume. For example, in some embodiments, dendrimers associated with (e.g., conjugated to) iron oxide NPs of the present invention are used to determine the stage of cancer (e.g., using a GLEASON grade or TNM staging). In other embodiments, the present invention provides targeting and identification of cancer cells (e.g., cancerous prostate cells) or tissues that permits the detection of the cancer cells and tissue in any region (e.g., in breast tissue, colon tissue, prostate, epithelium, lung, etc.) of the subject. For example, in some embodiments, the present invention detects and/or targets cancerous cells or tissue in a region outside of the primary site of cancer including, but not limited to, the vasculature and lymph nodes (e.g., the periprostatic, obturator, external iliac, hypogastric, common iliac and periaortic nodes). In some embodiments, detection (e.g., using a dendrimer of the present invention associated with (e.g., conjugated to) iron oxide NPs) of a cancerous cell outside of a primary cancer site is indicative of metastasis. Thus, in some embodiments, the present invention provides diagnostic information regarding metastasis and progression of a primary cancer. In preferred embodiments, dendrimers comprising (e.g., associated with (e.g., conjugated to)) iron oxide NPs, that target and identify cancer cells (e.g., metastatic and solid tissue cancer cells) further comprise a therapeutic agent for treatment of and/or eradication of the cancer cells. In some embodiments, a targeting moiety (e.g., folic acid, NAALADase inhibitor ligand, RGD peptide, or other targeting moiety described herein) present on the dendrimers comprising (e.g., associated with (e.g., conjugated to)) iron oxide NPs of the present invention targets cells expressing a ligand for the targeting moiety (e.g., folic acid receptor, PSMA, etc.) thereby permitting targeting, identification and treatment with little to no toxicity to surrounding healthy cells and tissue.

For example, following radical prostatectomy, PSMA levels become undetectable and rise when the tumor recurs. Thus, in some embodiments, the present invention provides compositions (e.g., dendrimers comprising iron oxide NPs) and methods for identifying cancer cells (e.g., prostate cancer cells) or tissue (e.g., prostate tumors) in the circulation and/or in the bone marrow of patients with all stages of cancer (e.g., prostate cancer, lung cancer, colon cancer, epithelial cancer, etc.). In some embodiments, dendrimers comprising iron oxide NPs further comprise a targeting agent, an imaging agent and/or a therapeutic agent.

The present invention is not limited by the type of therapeutic agent delivered via a dendrimer of the present invention. For example, a therapeutic may be any agent selected from the group comprising, but not limited to, a chemotherapeutic agent, an anti-oncogenic agent, an anti-angiogenic agent, a tumor suppressor agent, an anti-microbial agent, or an expression construct comprising a nucleic acid encoding a therapeutic protein. Illustrative examples of these types of agents are described herein.

The dendrimers of the present invention find use in the detection and treatment of a variety of cancers. Indeed, the present invention is not limited by the type of cancer to be treated. Thus, in some embodiments, the present invention provides compositions comprising dendrimers comprising iron oxide NPs for the targeting and identification of angiogenesis associated with cancers (e.g., carcinomas). For example, in some embodiments, a dendrimer comprising iron oxide NPs of the present invention further comprises a targeting agent (e.g., folic acid moiety) that associates with high affinity to a targeting agent ligand (e.g., receptor) on a cancer cell (e.g., carcinoma cells and/or solid tumor cells). In some embodiments, dendrimers comprising iron oxide NPs and a targeting agent, that target and identify cancer cells and/or angiogenesis associated with cancer, further comprise a therapeutic agent that inhibits angiogenesis thereby treating the cancer. In some embodiments, treatment with dendrimers comprising iron oxide NPs and an anti-angiogenic agent are used in combination with other dendrimers of the present invention, with other chemotherapeutic treatments, and/or as a treatment following surgical removal of a tumor or cancerous tissue. In preferred embodiments, a targeting moiety (e.g., folic acid or other targeting moiety described herein) possesses a high affinity for ligands (e.g., receptors or other types of proteins or molecules) present on cancer cell possessing such ligands thereby permitting the targeting, identification and treatment of disease (e.g., cancer) with little to no toxicity to surrounding healthy cells and tissue.

Dendrimers comprising iron oxide NPs of the present invention are not limited by the type of anti-angiogenic agent used. Indeed, a variety of anti-angiogenic agents are contemplated to be useful in the compositions of the present invention including, but not limited to, Batimastat, Marimastat, AG3340, Neovastat, PEX, TIMP-1, -2, -3, -4, PAI-1, -2, uPA Ab, uPAR Ab, Amiloride, Minocycline, tetracyclines, steroids, cartilage-derived TIMP, αᵥβ₃ Ab: LM609 and Vitaxin, RGD containing peptides, αvβ5 Ab, Endostatin, Angiostatin, aaAT, IFN-α, IFN-γ, IL-12, nitric oxide synthase inhibitors, TSP-1, TNP-470, Combretastatin A4, Thalidomide, Linomide, IFN-α, PF-4, prolactin fragment, Suramin and analogues, PPS, distamycin A analogues, FGF-2 Ab, antisense-FGF-2, Protamine, SU5416, soluble Flt-1, dominant-negative Flk-1, VEGF receptor ribosymes, VEGF Ab, Aspirin, NS-398, 6-AT, 6A5BU, 7-DX, Genistein, Lavendustin A, Ang-2, batimastat, marimastat, anti-αvβ3 monoclonal antibody (LM609) thrombospondin-1 (TSP-1) Angiostatin, endostatin, TNP-470, Combretastatin A-4, Anti-VEGF antibodies, soluble Flk-1, Flt-1 receptors, inhibitors of tyrosine kinase receptors, SU5416, heparin-binding growth factors, pentosan polysulfate, platelet-derived endothelial cell growth factor/Thymidine phosphorylase (PD-ECGF/TP), cox (e.g., cox-1 an cox-2) inhibitors (e.g., Celebrex and Vioxx), DT385, Tissue inhibitor of metalloprotease (TIMP-1, TIMP-2), Zinc, Plasminogen activator-inhibitor-1 (PAI-1), p53 Rb, Interleukin-10 Interleukin-12, Angiopoietin-2, Angiotensin, Angiotensin II (AT2 receptor), Caveolin-1, caveolin-2, Angiopoietin-2, Angiotensin, Angiotensin II (AT2 receptor), Caveolin-1, caveolin-2, Endostatin, Interferon-alpha, Isoflavones, Platelet factor-4, Prolactin (16 Kd fragment), Thrombospondin, Troponin-1, Bay 12-9566, AG3340, CGS 27023A, CGS 27023A, COL-3, (Neovastat), BMS-275291, Penicillamine, TNP-470 (fumagillin derivative), Squalamine, Combretastatin, Endostatin, Penicillamine, Farnesyl Transferase Inhibitor (FTI), -L-778,123, —SCH66336, -R115777, anti-VEGF antibody, Thalidomide, SU5416, Ribozyme, Angiozyme, SU6668, PTK787/ZK22584, Interferon-alpha, Interferon-alpha, Suramin, Vitaxin, EMD121974, Penicillamine, Tetrathiomolybdate, Captopril, serine protease inhibitors, CAI, ABT-627, CM101/ZDO101, Interleukin-12, IM862, PNU-145156E, those described in U.S. Patent App. No. 20050123605, herein incorporated by reference in its entirety, and fragments or portions of the above that retain anti-angiogenic (e.g., angiostatic or inhibitory properties).

In preferred embodiments, the compositions and methods of the present invention are used in treatment and/or monitoring during cancer therapy. However, the systems and compositions of the present invention find use in the treatment and monitoring of a variety of disease states or other physiological conditions, and the present invention is not limited to use with any particular disease state or condition. Other disease states that find particular use with the present invention include, but are not limited to, cardiovascular disease, viral disease, inflammatory disease, and other proliferative disorders.

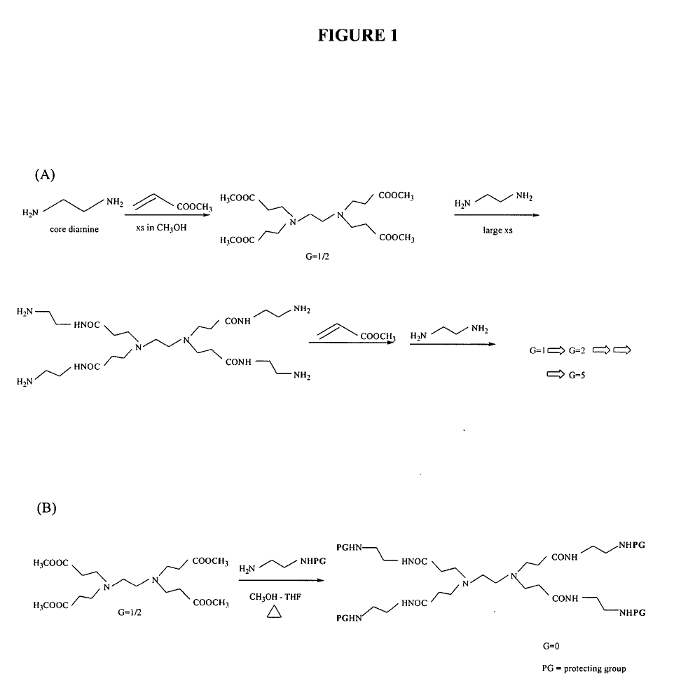

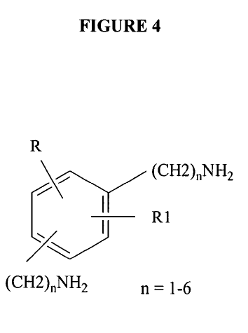

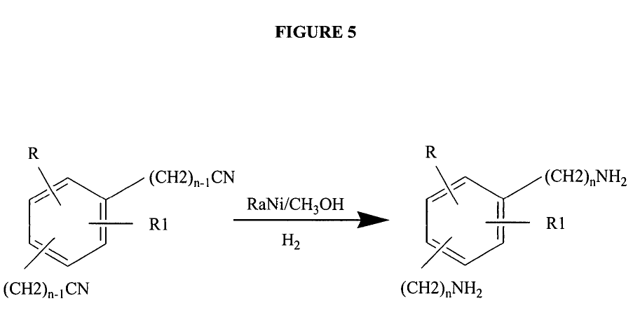

In some embodiments, the present invention provides a partially acetylated generation 5 (G5) polyamideamine (PAMAM), dendrimer comprising iron oxide NPs (See, e.g., Examples 18 and 19). In other preferred embodiments, the present invention provides methods of manufacturing a G5 dendrimer (See, e.g., Examples 2) comprising iron oxide NPs and a method of manufacturing a dendrimer comprising iron oxide NPs comprising a protected core diamine (See, e.g., FIGS. 1-5 and Examples 18 and 19).

Some embodiments of the present invention provide compositions comprising dendrimers and iron oxide NPs further comprising one or more functional groups, the functional groups including, but not limited to, therapeutic agents, biological monitoring components, biological imaging components, targeting components, and components to identify the specific signature of cellular abnormalities. As such, in some embodiments, a therapeutic nanodevice (e.g., a composition comprising a dendrimer) of the present invention is made up of individual dendrimers, each with one or more functional groups being specifically conjugated with or covalently linked to the dendrimer (See, e.g., Examples 2 and 6). In preferred embodiment, at least one of the functional groups is conjugated to the dendrimer via an ester bond (See, e.g., Example 7).

The following discussion describes individual component parts of the dendrimer and methods of making and using the same in some embodiments of the present invention. To illustrate the design and use of the systems and compositions of the present invention, the discussion focuses on specific embodiments of the use of the compositions in the treatment and monitoring of cancer. These specific embodiments are intended only to illustrate certain preferred embodiments of the present invention and are not intended to limit the scope thereof. In these embodiments, the dendrimers comprising (e.g., associated with (e.g., conjugated to)) iron oxide NPs of the present invention target the neoplastic cells through cell-surface moieties (e.g., folic acid receptor) and are taken up by the tumor cell (e.g., through receptor mediated endocytosis) (See, e.g., Examples 18 and 19). Although an understanding of the mechanism is not necessary to practice the present invention and the present invention is not limited to any particular mechanism of action, in some embodiments, the iron oxide NP component of the dendrimer allows cancerous cells (e.g., neoplastic cells (e.g., KB cells)) to be imaged (e.g., through the use of MRI).

In some embodiments, the release of a therapeutic agent is facilitated by the therapeutic component being attached to a labile protecting group, such as, for example, cisplatin or methotrexate being attached to a photolabile protecting group that becomes released by laser light directed at cells emitting a color of fluorescence (e.g., cells that have taken up dendrimers comprising iron oxide NPs and an targeting agent e.g., folic acid or other targeting agent described herein). In some embodiments, the therapeutic device also may have a component to monitor the response of the tumor to therapy. For example, where a therapeutic agent of the dendrimer induces apoptosis of a target cell (e.g., a cancer cell (e.g., a prostate cancer cell)), the caspase activity of the cells may be used to activate a green fluorescence. This allows apoptotic cells to turn orange, (combination of red and green) while residual cells remain red. Any normal cells that are induced to undergo apoptosis in collateral damage fluoresce green.

As is clear from the above example, the use of the compositions of the present invention facilitates non-intrusive sensing, signaling, and intervention for cancer and other diseases and conditions. Since specific protocols of molecular alterations in cancer cells are identified using this technique, non-intrusive sensing through the dendrimers is achieved and may then be employed automatically against various tumor phenotypes.

### I. Dendrimers

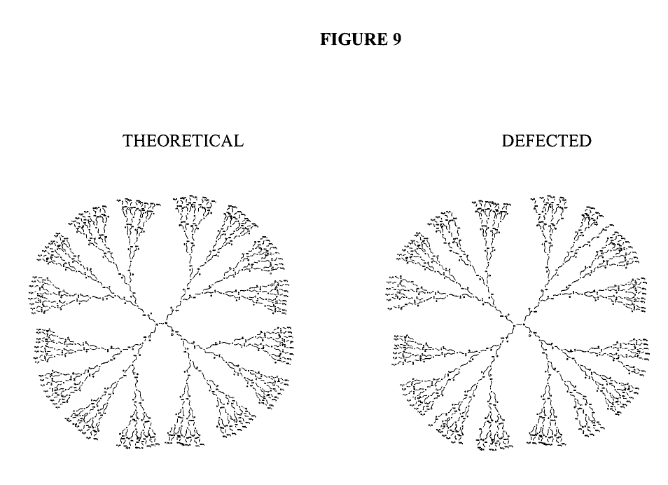

In preferred embodiments, compositions of the present invention comprise dendrimers (See, e.g., FIGS. 1-5 and Example 2) wherein the dendrimers further comprise iron oxide NPs (See, e.g., Example 18). Dendrimeric polymers have been described extensively (See, e.g., Tomalia, Advanced Materials 6:529 (1994); Angew, Chem. Int. Ed. Engl., 29:138 (1990); incorporated herein by reference in their entireties). Dendrimer polymers are synthesized as defined spherical structures typically ranging from 1 to 20 nanometers in diameter. Methods for manufacturing a G5 PAMAM dendrimer with a protected core is shown (FIGS. 1-5). In preferred embodiments, the protected core diamine is NH₂—CH2-CH2-NHPG. Molecular weight and the number of terminal groups increase exponentially as a function of generation (the number of layers) of the polymer (See, e.g., FIG. 9). Different types of dendrimers can be synthesized based on the core structure that initiates the polymerization process (See e.g., FIGS. 1-5).

The dendrimer core structures dictate several characteristics of the molecule such as the overall shape, density and surface functionality (See, e.g., Tomalia et al., Chem. Int. Ed. Engl., 29:5305 (1990)). Spherical dendrimers can have ammonia as a trivalent initiator core or ethylenediamine (EDA) as a tetravalent initiator core (See, e.g., FIG. 9). Recently described rod-shaped dendrimers (See, e.g., Yin et al., J. Am. Chem. Soc., 120:2678 (1998)) use polyethyleneimine linear cores of varying lengths; the longer the core, the longer the rod. Dendritic macromolecules are available commercially in kilogram quantities and are produced under current good manufacturing processes (GMP) for biotechnology applications.

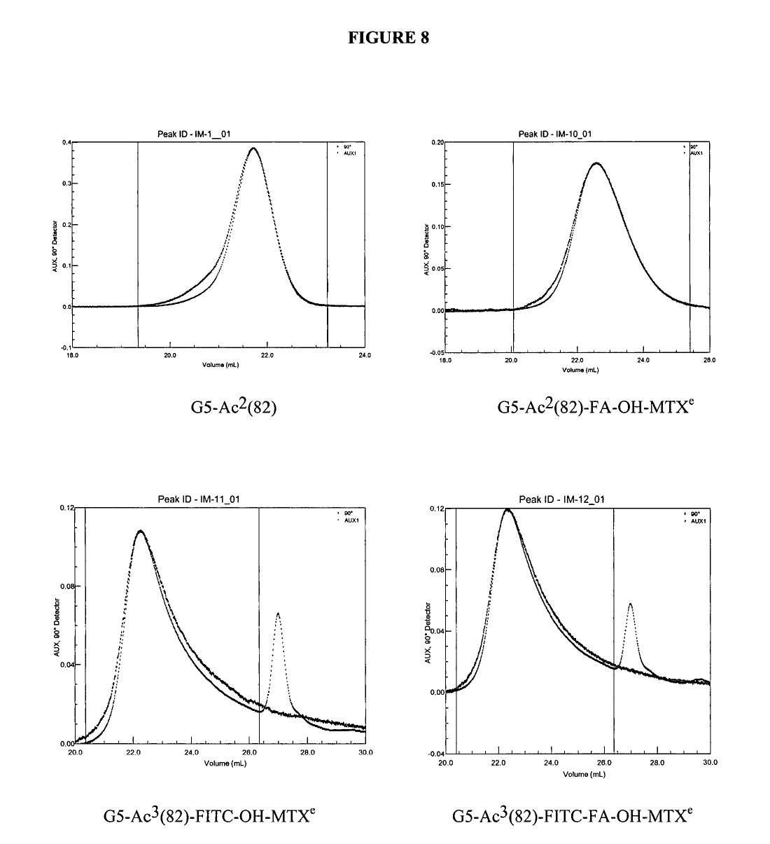

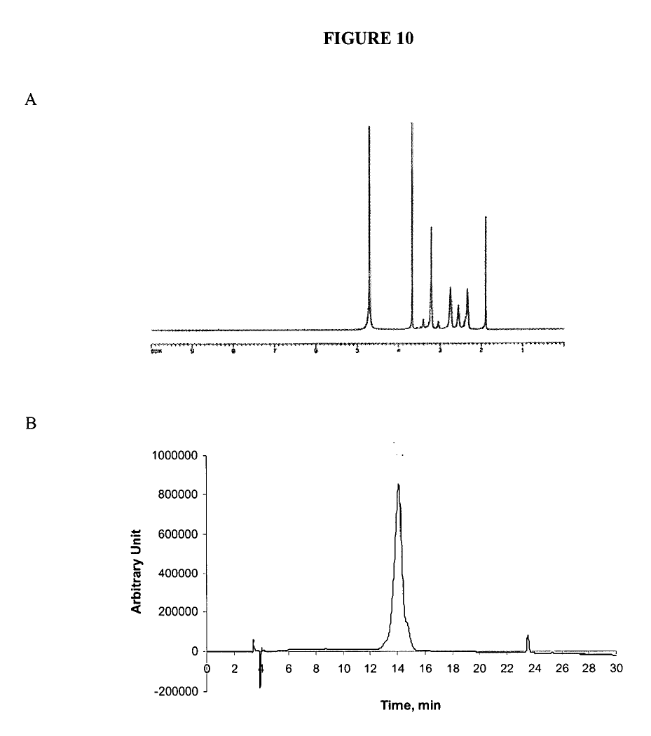

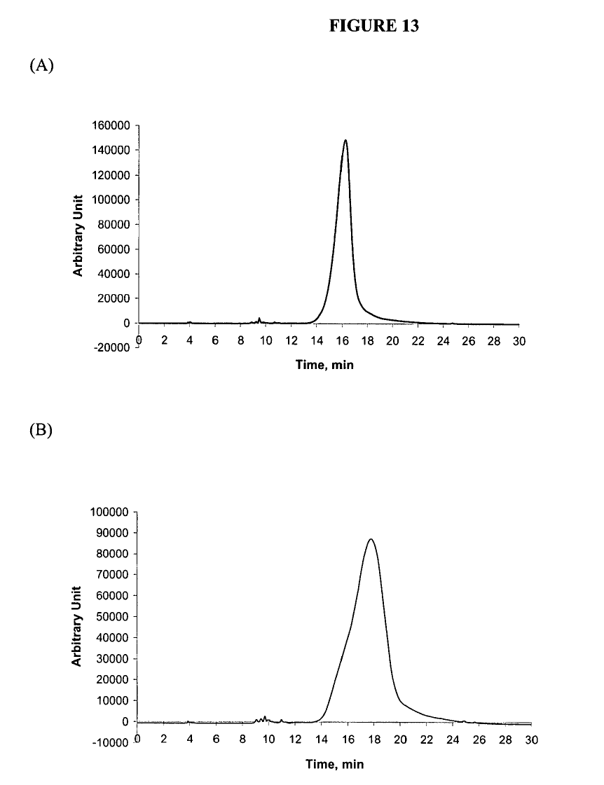

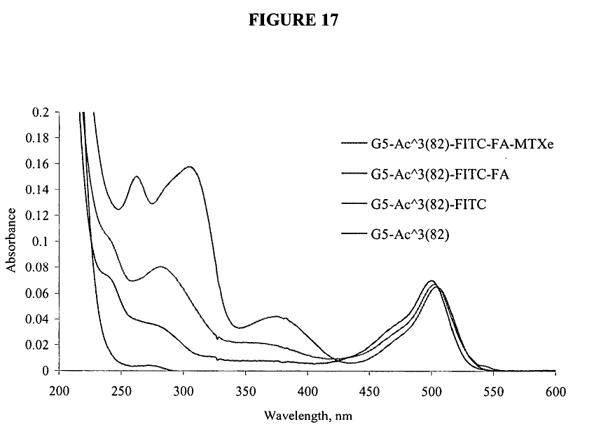

Dendrimers may be characterized by a number of techniques including, but not limited to, electrospray-ionization mass spectroscopy, ¹³C nuclear magnetic resonance spectroscopy, ¹H nuclear magnetic resonance spectroscopy (See, e.g., Example 5, FIG. 10(A) and Example 7, FIG. 14), high performance liquid chromatography (See, e.g., Example 5, FIG. 10(B); and Example 6, FIG. 13), size exclusion chromatography with multi-angle laser light scattering (See, e.g., Example 4, FIG. 8), ultraviolet spectrophotometry (See, e.g., Example 8, FIG. 17), capillary electrophoresis and gel electrophoresis. These tests assure the uniformity of the polymer population and are important for monitoring quality control of dendrimer manufacture for GMP applications and in vivo usage.

Numerous U.S. patents describe methods and compositions for producing dendrimers. Examples of some of these patents are given below in order to provide a description of some dendrimer compositions that may be useful in the present invention, however it should be understood that these are merely illustrative examples and numerous other similar dendrimer compositions could be used in the present invention.

U.S. Pat. No. 4,507,466, U.S. Pat. No. 4,558,120, U.S. Pat. No. 4,568,737, and U.S. Pat. No. 4,587,329 each describe methods of making dense star polymers with terminal densities greater than conventional star polymers. These polymers have greater/more uniform reactivity than conventional star polymers, i.e. 3rd generation dense star polymers. These patents further describe the nature of the amidoamine dendrimers and the 3-dimensional molecular diameter of the dendrimers.

U.S. Pat. No. 4,631,337 describes hydrolytically stable polymers. U.S. Pat. No. 4,694,064 describes rod-shaped dendrimers. U.S. Pat. No. 4,713,975 describes dense star polymers and their use to characterize surfaces of viruses, bacteria and proteins including enzymes. Bridged dense star polymers are described in U.S. Pat. No. 4,737,550. U.S. Pat. No. 4,857,599 and U.S. Pat. No. 4,871,779 describe dense star polymers on immobilized cores useful as ion-exchange resins, chelation resins and methods of making such polymers.

U.S. Pat. No. 5,338,532 is directed to starburst conjugates of dendrimer(s) in association with at least one unit of carried agricultural, pharmaceutical or other material. This patent describes the use of dendrimers to provide means of delivery of high concentrations of carried materials per unit polymer, controlled delivery, targeted delivery and/or multiple species such as e.g., drugs antibiotics, general and specific toxins, metal ions, radionuclides, signal generators, antibodies, interleukins, hormones, interferons, viruses, viral fragments, pesticides, and antimicrobials.

U.S. Pat. No. 6,471,968 describes a dendrimer complex comprising covalently linked first and second dendrimers, with the first dendrimer comprising a first agent and the second dendrimer comprising a second agent, wherein the first dendrimer is different from the second dendrimer, and where the first agent is different than the second agent.

Other useful dendrimer type compositions are described in U.S. Pat. No. 5,387,617, U.S. Pat. No. 5,393,797, and U.S. Pat. No. 5,393,795 in which dense star polymers are modified by capping with a hydrophobic group capable of providing a hydrophobic outer shell. U.S. Pat. No. 5,527,524 discloses the use of amino terminated dendrimers in antibody conjugates.

The use of dendrimers as metal ion carriers is described in U.S. Pat. No. 5,560,929. U.S. Pat. No. 5,773,527 discloses non-crosslinked polybranched polymers having a comb-burst configuration and methods of making the same. U.S. Pat. No. 5,631,329 describes a process to produce polybranched polymer of high molecular weight by forming a first set of branched polymers protected from branching; grafting to a core; deprotecting first set branched polymer, then forming a second set of branched polymers protected from branching and grafting to the core having the first set of branched polymers, etc.

U.S. Pat. No. 5,902,863 describes dendrimer networks containing lipophilic organosilicone and hydrophilic polyanicloamine nanscopic domains. The networks are prepared from copolydendrimer precursors having PAMAM (hydrophilic) or polyproyleneimine interiors and organosilicon outer layers. These dendrimers have a controllable size, shape and spatial distribution. They are hydrophobic dendrimers with an organosilicon outer layer that can be used for specialty membrane, protective coating, composites containing organic organometallic or inorganic additives, skin patch delivery, absorbants, chromatography personal care products and agricultural products.

U.S. Pat. No. 5,795,582 describes the use of dendrimers as adjuvants for influenza antigen. Use of the dendrimers produces antibody titer levels with reduced antigen dose. U.S. Pat. No. 5,898,005 and U.S. Pat. No. 5,861,319 describe specific immunobinding assays for determining concentration of an analyte. U.S. Pat. No. 5,661,025 provides details of a self-assembling polynucleotide delivery system comprising dendrimer polycation to aid in delivery of nucleotides to target site. This patent provides methods of introducing a polynucleotide into a eukaryotic cell in vitro comprising contacting the cell with a composition comprising a polynucleotide and a dendrimer polycation non-covalently coupled to the polynucleotide.

Dendrimer-antibody conjugates for use in in vitro diagnostic applications has previously been demonstrated (See, e.g., Singh et al., Clin. Chem., 40:1845 (1994)), for the production of dendrimer-chelant-antibody constructs, and for the development of boronated dendrimer-antibody conjugates (for neutron capture therapy); each of these latter compounds may be used as a cancer therapeutic (See, e.g., Wu et al., Bioorg. Med. Chem. Lett., 4:449 (1994); Wiener et al., Magn. Reson. Med. 31:1 (1994); Barth et al., Bioconjugate Chem. 5:58 (1994); and Barth et al.).

Some of these conjugates have also been employed in the magnetic resonance imaging of tumors (See, e.g., Wu et al., (1994) and Wiener et al., (1994), supra). Results from this work have documented that, when administered in vivo, antibodies can direct dendrimer-associated therapeutic agents to antigen-bearing tumors. Dendrimers also have been shown to specifically enter cells and carry either chemotherapeutic agents or genetic therapeutics. In particular, studies show that cisplatin encapsulated in dendrimer polymers has increased efficacy and is less toxic than cisplatin delivered by other means (See, e.g., Duncan and Malik, Control R^{e1}. Bioact. Mater. 23:105 (1996)).

Dendrimers have also been conjugated to fluorochromes or molecular beacons and shown to enter cells. They can then be detected within the cell in a manner compatible with sensing apparatus for evaluation of physiologic changes within cells (See, e.g., Baker et al., Anal. Chem. 69:990 (1997)). Finally, dendrimers have been constructed as differentiated block copolymers where the outer portions of the molecule may be digested with either enzyme or light-induced catalysis (See, e.g., Urdea and Hom, Science 261:534 (1993)). This allows the controlled degradation of the polymer to release therapeutics at the disease site and provides a mechanism for an external trigger to release the therapeutic agents.

The present invention provides dendrimers comprising iron oxide NPs wherein one or more functional groups, each with a specific functionality, are provided in a single dendrimer (See, e.g., Examples 7, 8 and 18). For example, a preferred composition of the present invention comprises a partially acetylated generation 5 (G5) PAMAM dendrimer comprising iron oxide NPs further comprising a targeting agent, and an imaging agent, the targeting agent comprises folic acid, and the imaging agent comprises fluorescein isothiocyanate (See, e.g., Examples 18). In some embodiments, the dendrimer further comprises a therapeutic agent (e.g., methotrexate or cisplatin). Hence, the present invention provides a single, multifunction dendrimer. In some embodiments, any one of the above functional groups (e.g., therapeutic agents) is provided in multiple copies on a single dendrimer. For example, in some embodiments, a single dendrimer comprises 2-100 copies of a single functional group (e.g., a therapeutic agent such as methotrexate or a targeting agent such as folic acid).

### II. Therapeutic Agents

A wide range of therapeutic agents find use with the present invention. Any therapeutic agent that can be associated with a dendrimer may be delivered using the methods, systems, and compositions of the present invention. To illustrate delivery of therapeutic agents, the following discussion focuses mainly on the delivery of methotrexate, cisplatin and taxol for the treatment of cancer. Also discussed are various photodynamic therapy compounds. However, the present invention is not limited to solely to the use of these exemplary agents. Indeed, a wide variety of agents (e.g., therapeutic agents) find use with the dendrimers of the present invention (e.g., as described herein).

i. Methotrexate, Cisplatin and Taxol

The cytotoxicity of methotrexate depends on the duration for which a threshold intracellular level is maintained (Levasseur et al., Cancer Res 58, 5749 (1998); Goldman & Matherly, Pharmacol Ther 28, 77 (1985)). Cells contain high concentrations of DHFR, and, to shut off the DHFR activity completely, anti-folate levels six orders of magnitude higher than the Ki for DHFR is required (Sierrra & Goldman, Seminars in Oncology 26, 11 (1999)). Furthermore, less than 5% of the enzyme activity is sufficient for full cellular enzymatic function (White & Goldman, Biol Chem 256, 5722 (1981)). Cisplatin and Taxol have a well-defined action of inducing apoptosis in tumor cells (See e.g., Lanni et al., Proc. Natl. Acad. Sci., 94:9679 (1997); Tortora et al., Cancer Research 57:5107 (1997); and Zaffaroni et al., Brit. J. Cancer 77:1378 (1998)). However, treatment with these and other chemotherapeutic agents is difficult to accomplish without incurring significant toxicity. The agents currently in use are generally poorly water soluble, quite toxic, and given at doses that affect normal cells as wells as diseased cells. For example, paclitaxel (Taxol), one of the most promising anticancer compounds discovered, is poorly soluble in water.

Paclitaxel has shown excellent antitumor activity in a wide variety of tumor models such as the B16 melanoma, L1210 leukemias, MX-1 mammary tumors, and CS-1 colon tumor xenografts. However, the poor aqueous solubility of paclitaxel presents a problem for human administration. Accordingly, currently used paclitaxel formulations require a cremaphor to solubilize the drug. The human clinical dose range is 200-500 mg. This dose is dissolved in a 1:1 solution of ethanol:cremaphor and diluted to one liter of fluid given intravenously. The cremaphor currently used is polyethoxylated castor oil. It is given by infusion by dissolving in the cremaphor mixture and diluting with large volumes of an aqueous vehicle. Direct administration (e.g., subcutaneous) results in local toxicity and low levels of activity. Thus, there is a need for more efficient and effective delivery systems for these chemotherapeutic agents.

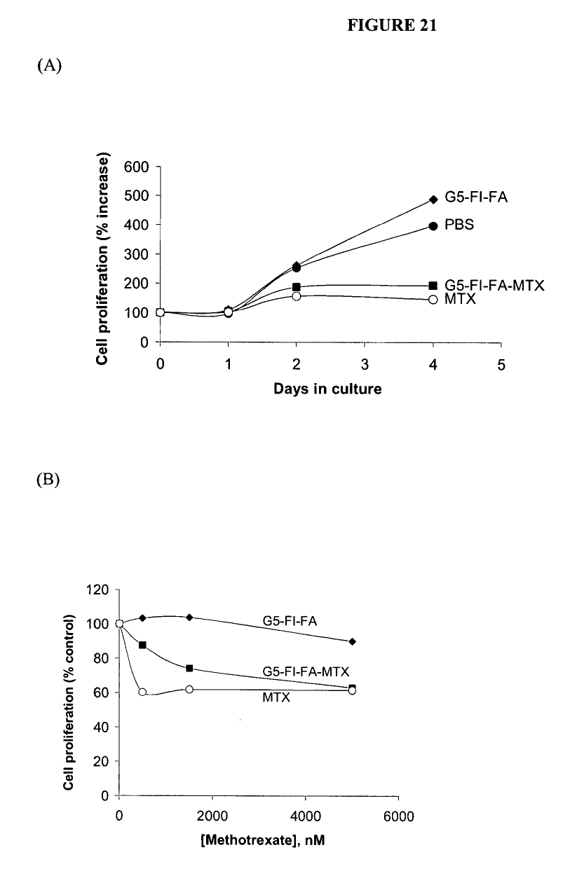

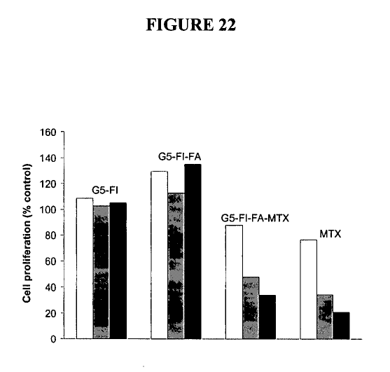

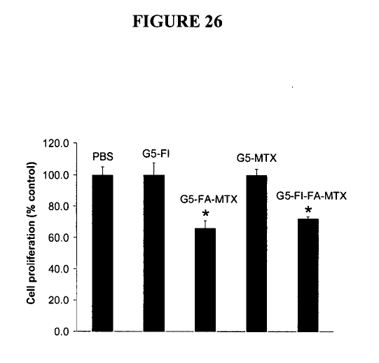

The present invention overcomes these problems by providing methods and compositions for specific drug delivery. The present invention also provides the ability to administer combinations of agents (e.g., two or more different therapeutic agents) to produce an additive effect. The use of multiple agent may be used to counter disease resistance to any single agent. For example, resistance of some cancers to single drugs (taxol) has been reported (Yu et al., Molecular Cell. 2:581 (1998)). Experiments conducted during the development of the present invention have demonstrated that methotrexate, conjugated to dendrimers, is able to efficiently kill cancer cells (See, Example 10, FIGS. 21 and 22, and Example 12, FIG. 26). Thus, in some embodiments, the present invention provides a dendrimer comprising iron oxide NPs further comprising a chemotherapeutic agent (e.g., the therapeutic agent methotrexate). In some embodiments, a dendrimer comprising iron oxide NPs and methotrexate is used to target and treat (e.g., kill) cancer cells (e.g., prostate cancer cells) within a subject. The present invention is contemplated to be useful for treating a subject with any stage of cancer (e.g., prostate cancer). In some embodiments, compositions of the present invention can be used prophylactically.

The present invention also provides the opportunity to monitor therapeutic success following delivery of a therapeutic agent (e.g., methotrexate, cisplatin and/or Taxol) to a subject. For example, measuring the ability of these drugs to induce apoptosis in vitro is reported to be a marker for in vivo efficacy (Gibb, Gynecologic Oncology 65:13 (1997)). Therefore, in addition to the targeted delivery of a therapeutic agent (e.g., either one, two or all of the above mentioned drugs) to provide effective anti-tumor therapy and reduction of toxicity, the effectiveness of the therapy can be gauged by a biological monitoring agent of the present invention (e.g., that monitor the induction of apoptosis). It is contemplated that dendrimers comprising iron oxide NPs further comprising a therapeutic agent and/or imaging agents and/or biological imaging agents are active against a wide-range of tumor types including, but not limited to, prostate cancer, breast cancer, colon cancer, lung cancer, epithelial cancer, etc.

Although the above discussion describes the specific therapeutic agents methotrexate, cisplatin and Taxol, any pharmaceutical that is routinely used in a cancer therapy context finds use in the present invention. In treating cancer according to the invention, the therapeutic component of the dendrimer may comprise compounds including, but not limited to, adriamycin, 5-fluorouracil, etoposide, camptothecin, actinomycin-D, mitomycin C, or more preferably, cisplatin. The agent may be prepared and used as a combined therapeutic composition, or kit, by combining it with an immunotherapeutic agent, as described herein.

In some embodiments of the present invention, the dendrimer is contemplated to comprise one or more agents that directly cross-link nucleic acids (e.g., DNA) to facilitate DNA damage leading to a synergistic, antineoplastic agents of the present invention. Agents such as cisplatin, and other DNA alkylating agents may be used. Cisplatin has been widely used to treat cancer, with efficacious doses used in clinical applications of 20 mg/m² for 5 days every three weeks for a total of three courses. The dendrimers may be delivered via any suitable method, including, but not limited to, injection intravenously, subcutaneously, intratumorally, intraperitoneally, or topically (e.g., to mucosal surfaces).

Agents that damage DNA also include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 Mg/M² at 21 day intervals for adriamycin, to 35-50 Mg/M² for etoposide intravenously or double the intravenous dose orally.

Agents that disrupt the synthesis and fidelity of nucleic acid precursors and subunits also lead to DNA damage and find use as chemotherapeutic agents in the present invention. A number of nucleic acid precursors have been developed. Particularly useful are agents that have undergone extensive testing and are readily available. As such, agents such as 5-fluorouracil (5-FU) are preferentially used by neoplastic tissue, making this agent particularly useful for targeting to neoplastic cells. The doses delivered may range from 3 to 15 mg/kg/day, although other doses may vary considerably according to various factors including stage of disease, amenability of the cells to the therapy, amount of resistance to the agents and the like.

The anti-cancer therapeutic agents that find use in the present invention are those that are amenable to incorporation into dendrimer structures or are otherwise associated with dendrimer structures such that they can be delivered into a subject, tissue, or cell without loss of fidelity of its anticancer effect. For a more detailed description of cancer therapeutic agents such as a platinum complex, verapamil, podophyllotoxin, carboplatin, procarbazine, mechlorethamine, cyclophosphamide, camptothecin, ifosfamide, melphalan, chlorambucil, bisulfan, nitrosurea, adriamycin, dactinomycin, daunorubicin, doxorubicin, bleomycin, plicomycin, mitomycin, etoposide (VP16), tamoxifen, taxol, transplatinum, 5-fluorouracil, vincristin, vinblastin and methotrexate and other similar anti-cancer agents, those of skill in the art are referred to any number of instructive manuals including, but not limited to, the Physician's Desk reference and to Goodman and Gilman's “Pharmaceutical Basis of Therapeutics” ninth edition, Eds. Hardman et al., 1996.

In some embodiments, the drugs are preferably attached to the dendrimers with photocleavable linkers. For example, several heterobifunctional, photocleavable linkers that find use with the present invention are described by Ottl et al. (Ottl et al., Bioconjugate Chem., 9:143 (1998)). These linkers can be either water or organic soluble. They contain an activated ester that can react with amines or alcohols and an epoxide that can react with a thiol group. In between the two groups is a 3,4-dimethoxy-6-nitrophenyl photoisomerization group, which, when exposed to near-ultraviolet light (365 nm), releases the amine or alcohol in intact form. Thus, the therapeutic agent, when linked to the compositions of the present invention using such linkers, may be released in biologically active or activatable form through exposure of the target area to near-ultraviolet light.

In a preferred embodiment, methotrexate is conjugated to the dendrimer via an ester bond (See, e.g., Example 7). In an exemplary embodiment, the alcohol group of taxol is reacted with the activated ester of the organic-soluble linker. This product in turn is reacted with the partially-thiolated surface of appropriate dendrimers (the primary amines of the dendrimers can be partially converted to thiol-containing groups by reaction with a sub-stoichiometric amount of 2-iminothiolano). In the case of cisplatin, the amino groups of the drug are reacted with the water-soluble form of the linker. If the amino groups are not reactive enough, a primary amino-containing active analog of cisplatin, such as Pt(II) sulfadiazine dichloride (Pasani et al., Inorg. Chim. Acta 80:99 (1983) and Abel et al, Eur. J. Cancer 9:4 (1973)) can be used. Thus conjugated, the drug is inactive and will not harm normal cells. When the conjugate is localized within tumor cells, it is exposed to laser light of the appropriate near-UV wavelength, causing the active drug to be released into the cell.

Similarly, in other embodiments of the present invention, the amino groups of cisplatin (or an analog thereof) is linked with a very hydrophobic photocleavable protecting group, such as the 2-nitrobenzyloxycarbonyl group (Pillai, V. N. R. Synthesis: 1-26 (1980)). With this hydrophobic group attached, the drug is loaded into and very preferentially retained by the hydrophobic cavities within the PAMAM dendrimer (See e.g., Esfand et al., Pharm. Sci., 2:157 (1996)), insulated from the aqueous environment. When exposed to near-LV light (about 365 nm), the hydrophobic group is cleaved, leaving the intact drug. Since the drug itself is hydrophilic, it diffuses out of the dendrimer and into the tumor cell, where it initiates apoptosis.

An alternative to photocleavable linkers are enzyme cleavable linkers. A number of photocleavable linkers have been demonstrated as effective anti-tumor conjugates and can be prepared by attaching cancer therapeutics, such as doxorubicin, to water-soluble polymers with appropriate short peptide linkers (See e.g., Vasey et al., Clin. Cancer Res., 5:83 (1999)). The linkers are stable outside of the cell, but are cleaved by thiol proteases once within the cell. In a preferred embodiment, the conjugate PK1 is used. As an alternative to the photocleavable linker strategy, enzyme-degradable linkers, such as Gly-Phe-Leu-Gly may be used.

The present invention is not limited by the nature of the therapeutic technique. For example, other conjugates that find use with the present invention include, but are not limited to, using conjugated boron dusters for BNCT (Capala et al., Bioconjugate Chem., 7:7 (1996)), the use of radioisotopes, and conjugation of toxins such as ricin to the nanodevice.

ii. Photodynamic Therapy

Photodynamic therapeutic agents may also be used as therapeutic agents in the present invention. In some embodiments, the dendrimeric compositions of the present invention containing photodynamic compounds are illuminated, resulting in the production of singlet oxygen and free radicals that diffuse out of the fiberless radiative effector to act on the biological target (e.g., tumor cells or bacterial cells). Some preferred photodynamic compounds include, but are not limited to, those that can participate in a type II photochemical reaction:

PS+hv PS*(1)

PS*(1)PS*(3)

PS*(3)+O₂ [symbol] PS+*O₂

*O₂+T [symbol] cytotoxity

where PS=photosensitizer, PS*(1)=excited singlet state of PS, PS*(3)=excited triplet state of PS, hv=light quantum, *O₂=excited singlet state of oxygen, and T=biological target. Other photodynamic compounds useful in the present invention include those that cause cytotoxity by a different mechanism than singlet oxygen production (e.g., copper benzochlorin, Selman, et al., Photochem. Photobiol., 57:681-85 (1993), incorporated herein by reference). Examples of photodynamic compounds that find use in the present invention include, but are not limited to Photofrin 2, phtalocyanins (See e.g., Brasseur et al., Photochem. Photobiol., 47:705-11 (1988)), benzoporphyrin, tetrahydroxyphenylporphyrins, naphtalocyanines (See e.g., Firey and Rodgers, Photochem. Photobiol., 45:535-38 (1987)), sapphyrins (See, e.g., Sessler et al., Proc. SPIE, 1426:318-29 (1991)), porphinones (See, e.g., Chang et al., Proc. SPIE, 1203:281-86 (1990)), tin etiopurpurin, ether substituted porphyrins (See, e.g., Pandey et al., Photochem. Photobiol., 53:65-72 (1991)), and cationic dyes such as the phenoxazines (See e.g., Cincotta et al., SPIE Proc., 1203:202-10 (1990)).

### III. Signature Identifying Agents

In certain embodiments, the nano-devices of the present invention contain one or more signature identifying agents that are activated by, or are able to interact with, a signature component (“signature”). In preferred embodiments, the signature identifying agent is an antibody, preferably a monoclonal antibody, that specifically binds the signature (e.g., cell surface molecule specific to a cell to be targeted).

In some embodiments of the present invention, tumor cells are identified. Tumor cells have a wide variety of signatures, including the defined expression of cancer-specific antigens such as Muc1, HER-2 and mutated p53 in breast cancer. These act as specific signatures for the cancer, being present in 30% (HER-2) to 70% (mutated p53) of breast cancers. In some embodiments, a dendrimer of the present invention comprises a monoclonal antibody that specifically binds to a mutated version of p53 that is present in breast cancer. In some embodiments, a dendrimer of the present invention comprises an antibody (e.g., monoclonal antibody) with high affinity for a signature including, but not limited to, Muc1 and HER-2.

In some embodiments of the present invention, cancer cells expressing susceptibility genes are identified. For example, in some embodiments, there are two breast cancer susceptibility genes that are used as specific signatures for breast cancer: BRCA1 on chromosome 17 and BRCA2 on chromosome 13. When an individual carries a mutation in either BRCA1 or BRCA2, they are at an increased risk of being diagnosed with breast or ovarian cancer at some point in their lives. These genes participate in repairing radiation-induced breaks in double-stranded DNA. It is thought that mutations in BRCA1 or BRCA2 might disable this mechanism, leading to more errors in DNA replication and ultimately to cancerous growth.

In addition, the expression of a number of different cell surface receptors find use as targets for the binding and uptake of the nano-device. Such receptors include, but are not limited to, EGF receptor, folate receptor, FGR receptor 2, and the like.

In some embodiments of the present invention, changes in gene expression associated with chromosomal abborations are the signature component. For example, Burkitt lymphoma results from chromosome translocations that involve the Myc gene. A chromosome translocation means that a chromosome is broken, which allows it to associate with parts of other chromosomes. The classic chromosome translocation in Burkitt lymophoma involves chromosome 8, the site of the Myc gene. This changes the pattern of Myc expression, thereby disrupting its usual function in controlling cell growth and proliferation.

In other embodiments, gene expression associated with colon cancer are identified as the signature component. Two key genes are known to be involved in colon cancer: MSH2 on chromosome 2 and MLH1 on chromosome 3. Normally, the protein products of these genes help to repair mistakes made in DNA replication. If the MSH2 and MLH1 proteins are mutated, the mistakes in replication remain unrepaired, leading to damaged DNA and colon cancer. MENI gene, involved in multiple endocrine neoplasia, has been known for several years to be found on chromosome 11, was more finely mapped in 1997, and serves as a signature for such cancers. In preferred embodiments of the present invention, an antibody specific for the altered protein or for the expressed gene to be detected is complexed with nanodevices of the present invention.

In yet another embodiment, adenocarcinoma of the colon has defined expression of CEA and mutated p53, both well-documented tumor signatures. The mutations of p53 in some of these cell lines are similar to that observed in some of the breast cancer cells and allows for the sharing of a p53 sensing component between the two nanodevices for each of these cancers (i.e., in assembling the nanodevice, dendrimers comprising the same signature identifying agent may be used for each cancer type). Both colon and breast cancer cells may be reliably studied using cell lines to produce tumors in nude mice, allowing for optimization and characterization in animals.

From the discussion above it is clear that there are many different tumor signatures that find use with the present invention, some of which are specific to a particular type of cancer and others which are promiscuous in their origin. The present invention is not limited to any particular tumor signature or any other disease-specific signature. For example, tumor suppressors that find use as signatures in the present invention include, but are not limited to, p53, Muc1, CEA, p16, p21, p27, CCAM, RB, APC, DCC, NF-1, NF-2, WT-1, MEN-1, MEN-II, p73, VHL, FCC and MCC.

### IV. Biological Imaging Component

In some embodiments of the present invention, the nanodevice comprises at least one dendrimer-based nanoscopic building block that can be readily imaged. The present invention is not limited by the nature of the imaging component used. In some embodiments of the present invention, imaging modules comprise surface modifications of quantum dots (See e.g., Chan and Nie, Science 281:2016 (1998)) such as zinc sulfide-capped cadmium selenide coupled to biomolecules (Sooklal, Adv. Mater., 10:1083 (1998)).

In some embodiments, the imaging module comprises dendrimers produced according to the “nanocomposite” concept (See, e.g., Balogh et al., Proc. of ACS PMSE 77:118 (1997) and Balogh and Tomalia, J. Am. Che. Soc., 120:7355 (1998)). In these embodiments, dendrimers are produced by reactive encapsulation, where a reactant is preorganized by the dendrimer template and is then subsequently immobilized in/on the polymer molecule by a second reactant. Size, shape, size distribution and surface functionality of these nanoparticles are determined and controlled by the dendritic macromolecules. These materials have the solubility and compatibility of the host and have the optical or physiological properties of the guest molecule (i.e., the molecule that permits imaging). While the dendrimer host may vary according to the medium, it is possible to load the dendrimer hosts with different compounds and at various guest concentration levels. Complexes and composites may involve the use of a variety of metals or other inorganic materials. The high electron density of these materials considerably simplifies the imaging by electron microscopy and related scattering techniques. In addition, properties of inorganic atoms introduce new and measurable properties for imaging in either the presence or absence of interfering biological materials. In some embodiments of the present invention, encapsulation of gold, silver, cobalt, iron atoms/molecules and/or organic dye molecules such as fluorescein are encapsulated into dendrimers for use as nanoscopi composite labels/tracers, although any material that facilitates imaging or detection may be employed. In a preferred embodiment, the imaging agent is fluorescein isothiocyanate

In some embodiments of the present invention, imaging is based on the passive or active observation of local differences in density of selected physical properties of the investigated complex matter. These differences may be due to a different shape (e.g., mass density detected by atomic force microscopy), altered composition (e.g. radiopaques detected by X-ray), distinct light emission (e.g., fluorochromes detected by spectrophotometry), different diffraction (e.g., electron-beam detected by TEM), contrasted absorption (e.g., light detected by optical methods), or special radiation emission (e.g., isotope methods), etc. Thus, quality and sensitivity of imaging depend on the property observed and on the technique used. The imaging techniques for cancerous cells have to provide sufficient levels of sensitivity to is observe small, local concentrations of selected cells. The earliest identification of cancer signatures requires high selectivity (i.e., highly specific recognition provided by appropriate targeting) and the highest possible sensitivity.

### A. Magnetic Resonance Imaging

Once the targeted nanodevice has attached to (or been internalized into) tumor cells, one or more modules on the device serve to image its location. Dendrimers have already been employed as biomedical imaging agents, perhaps most notably for magnetic resonance imaging (MRI) contrast enhancement agents (See e.g., Wiener et al., Mag. Reson. Med. 31:1 (1994); an example using PAMAM dendrimers). These agents are typically constructed by conjugating chelated paramagnetic ions, such as Gd(III)-diethylenetriaminepentaacetic acid (Gd(III)-DTPA), to water-soluble dendrimers. Other paramagnetic ions that may be useful in this context of the include, but are not limited to, gadolinium, manganese, copper, chromium, iron, cobalt, erbium, nickel, europium, technetium, indium, samarium, dysprosium, ruthenium, ytterbium, yttrium, and holmium ions and combinations thereof. In some embodiments of the present invention, the dendrimer is also conjugated to a targeting group, such as epidermal growth factor (EGF), to make the conjugate specifically bind to the desired cell type (e.g., in the case of EGF, EGFR-expressing tumor cells). In a preferred embodiment of the present invention, DTPA is attached to dendrimers via the isothiocyanate of DTPA as described by Wiener (Wiener et al., Mag. Reson. Med. 31:1 (1994)).

Dendrimeric MRI agents are particularly effective due to the polyvalency, size and architecture of dendrimers, which results in molecules with large proton relaxation enhancements, high molecular relaxivity, and a high effective concentration of paramagnetic ions at the target site. Dendrimeric gadolinium contrast agents have even been used to differentiate between benign and malignant breast tumors using dynamic MRI, based on how the vasculature for the latter type of tumor images more densely (Adam et al., Ivest. Rad. 31:26 (1996)). Thus, MRI provides a particularly useful imaging system of the present invention.

### B. Microscopic Imaging

Static structural microscopic imaging of cancerous cells and tissues has traditionally been performed outside of the patient. Classical histology of tissue biopsies provides a fine illustrative example, and has proven a powerful adjunct to cancer diagnosis and treatment. After removal, a specimen is sliced thin (e.g., less than 40 microns), stained, fixed, and examined by a pathologist. If images are obtained, they are most often 2-D transmission bright-field projection images. Specialized dyes are employed to provide selective contrast, which is almost absent from the unstained tissue, and to also provide for the identification of aberrant cellular constituents. Quantifying sub-cellular structural features by using computer-assisted analysis, such as in nuclear ploidy determination, is often confounded by the loss of histologic context owing to the thinness of the specimen and the overall lack of 3-D information. Despite the limitations of the static imaging approach, it has been invaluable to allow for the identification of neoplasia in biopsied tissue. Furthermore, its use is often the crucial factor in the decision to perform invasive and risky combinations of chemotherapy, surgical procedures, and radiation treatments, which are often accompanied by severe collateral tissue damage, complications, and even patient death.

The nanodevices of the present invention allow functional microscopic imaging of tumors and provide improved methods for imaging. The methods find use in vivo, in vitro, and ex vivo. For example, in one embodiment of the present invention, dendrimers of the present invention are designed to emit light or other detectable signals upon exposure to light. Although the labeled dendrimers may be physically smaller than the optical resolution limit of the microscopy technique, they become self-luminous objects when excited and are readily observable and measurable using optical techniques. In some embodiments of the present invention, sensing fluorescent biosensors in a microscope involves the use of tunable excitation and emission filters and multiwavelength sources (Farkas et al., SPEI 2678:200 (1997)). In embodiments where the imaging agents are present in deeper tissue, longer wavelengths in the Near-infrared (NMR) are used (See e.g., Lester et al., Cell Mol. Biol. 44:29 (1998)). Dendrimeric biosensing in the Near-IR has been demonstrated with dendrimeric biosensing antenna-like architectures (Shortreed et al., J. Phys. Chem., 101:6318 (1997)). Biosensors that find use with the present invention include, but are not limited to, fluorescent dyes and molecular beacons.

In some embodiments of the present invention, in vivo imaging is accomplished using functional imaging techniques. Functional imaging is a complementary and potentially more powerful techniques as compared to static structural imaging. Functional imaging is best known for its application at the macroscopic scale, with examples including functional Magnetic Resonance Imaging (fMRI) and Positron Emission Tomography (PET). However, functional microscopic imaging may also be conducted and find use in in vivo and ex vivo analysis of living tissue. Functional microscopic imaging is an efficient combination of 3-D imaging, 3-D spatial multispectral volumetric assignment, and temporal sampling: in short a type of 3-D spectral microscopic movie loop. Interestingly, cells and tissues autofluoresce. When excited by several wavelengths, providing much of the basic 3-D structure needed to characterize several cellular components (e.g., the nucleus) without specific labeling. Oblique light illumination is also useful to collect structural information and is used routinely. As opposed to structural spectral microimaging, functional spectral microimaging may be used with biosensors, which act to localize physiologic signals within the cell or tissue. For example, in some embodiments of the present invention, biosensor-comprising dendrimers of the present invention are used to image upregulated receptor families such as the folate or EGF classes. In such embodiments, functional biosensing therefore involves the detection of physiological abnormalities relevant to carcinogenesis or malignancy, even at early stages. A number of physiological conditions may be imaged using the compositions and methods of the present invention including, but not limited to, detection of nanoscopic dendrimeric biosensors for pH, oxygen concentration, Ca²+ concentration, and other physiologically relevant analytes.

### V. Biological Monitoring Component

The biological monitoring or sensing component of the nanodevice of the present invention is one that can monitor the particular response in the tumor cell induced by an agent (e.g., a therapeutic agent provided by the therapeutic component of the nanodevice). While the present invention is not limited to any particular monitoring system, the invention is illustrated by methods and compositions for monitoring cancer treatments. In preferred embodiments of the present invention, the agent induces apoptosis in cells and monitoring involves the detection of apoptosis. In particular embodiments, the monitoring component is an agent that fluoresces at a particular wavelength when apoptosis occurs. For example, in a preferred embodiment, caspase activity activates green fluorescence in the monitoring component. Apoptotic cancer cells, which have turned red as a result of being targeted by a particular signature with a red label, turn orange while residual cancer cells remain red. Normal cells induced to undergo apoptosis (e.g., through collateral damage), if present, will fluoresce green.

In these embodiments, fluorescent groups such as fluorescein are employed in the monitoring component. Fluorescein is easily attached to the dendrimer surface via the isothiocyanate derivatives, available from Molecular Probes, Inc. This allows the nanodevices to be imaged with the cells via confocal microscopy. Sensing of the effectiveness of the nanodevices is preferably achieved by using fluorogenic peptide enzyme substrates. For example, apoptosis caused by the therapeutic agents results in the production of the peptidase caspase-1 (ICE). Calbiochem sells a number of peptide substrates for this enzyme that release a fluorescent moiety. A particularly useful peptide for use in the present invention is: MCA-Tyr-Glu-Val-Asp-Gly-Trp-Lys-(DNP)-NH₂ (SEQ ID NO: 1) where MCA is the (7-methoxycoumarin-4-yl)acetyl and DNP is the 2,4-dinitrophenyl group (Talanian et al., J. Biol. Chem., 272: 9677 (1997)). In this peptide, the MCA group has greatly attenuated fluorescence, due to fluorogenic resonance energy transfer (FRET) to the DNP group. When the enzyme cleaves the peptide between the aspartic acid and glycine residues, the MCA and DNP are separated, and the MCA group strongly fluoresces green (excitation maximum at 325 nm and emission maximum at 392 nm).

In preferred embodiments of the present invention, the lysine end of the peptide is linked to the nanodevice, so that the MCA group is released into the cytosol when it is cleaved. The lysine end of the peptide is a useful synthetic handle for conjugation because, for example, it can react with the activated ester group of a bifunctional linker such as Mal-PEG-OSu. Thus the appearance of green fluorescence in the target cells produced using these methods provides a clear indication that apoptosis has begun (if the cell already has a red color from the presence of aggregated quantum dots, the cell turns orange from the combined colors).

Additional fluorescent dyes that find use with the present invention include, but are not limited to, acridine orange, reported as sensitive to DNA changes in apoptotic cells (Abrams et al., Development 117:29 (1993)) and cis-parinaric acid, sensitive to the lipid peroxidation that accompanies apoptosis (Hockenbery et al., Cell 75:241 (1993)). It should be noted that the peptide and the fluorescent dyes are merely exemplary. It is contemplated that any peptide that effectively acts as a substrate for a caspase produced as a result of apoptosis finds use with the present invention.

### VI. Targeting Agents

As described above, another component of the present invention is that the nanodevice compositions are able to specifically target a particular cell type (e.g., tumor cell). In some embodiments, the nanodevice targets neoplastic cells through a cell surface moiety and is taken into the cell through receptor mediated endocytosis.

In some embodiments of the present invention, targeting groups are conjugated to dendrimers with either short (e.g., direct coupling), medium (e.g. using small-molecule bifunctional linkers such as SPDP, sold by Pierce Chemical Company), or long (e.g., PEG bifunctional linkers, sold by Nektar, Inc.) linkages. Since dendrimers have surfaces with a large number of functional groups, more than one targeting group may be attached to each dendrimer. As a result, there are multiple binding events between the dendrimer and the target cell. In these embodiments, the dendrimers have a very high affinity for their target cells via this “cooperative binding” or polyvalent interaction effect.

For steric reasons, the smaller the ligands, the more can be attached to the surface of a dendrimer. Recently, Wiener reported that dendrimers with attached folic acid would specifically accumulate on the surface and within tumor cells expressing the high-affinity folate receptor (hFR) (See, e.g., Wiener et al., Invest. Radiol., 32:748 (1997)). The hFR receptor is expressed or upregulated on epithelial tumors, including breast cancers. Control cells lacking hFR showed no significant accumulation of folate-derivatized dendrimers. Folic acid can be attached to full generation PAMAM dendrimers via a carbodiimide coupling reaction. Folic acid is a good targeting candidate for the dendrimers, with its small size and a simple conjugation procedure.

Antibodies can be generated to allow for the targeting of antigens or immunogens (e.g., tumor, tissue or pathogen specific antigens) on various biological targets (e.g., pathogens, tumor cells, normal tissue). Such antibodies include, but are not limited to polyclonal, monoclonal, chimeric, single chain, Fab fragments, and an Fab expression library.

In some preferred embodiments, the antibodies recognize tumor specific epitopes (e.g., TAG-72 (Kjeldsen et al., Cancer Res. 48:2214-2220 (1988); U.S. Pat. Nos. 5,892,020; 5,892,019; and 5,512,443); human carcinoma antigen (U.S. Pat. Nos. 5,693,763; 5,545,530; and 5,808,005); TP1 and TP3 antigens from osteocarcinoma cells (U.S. Pat. No. 5,855,866); Thomsen-Friedenreich (TF) antigen from adenocarcinoma cells (U.S. Pat. No. 5,110,911); “KC-4 antigen” from human prostrate adenocarcinoma (U.S. Pat. Nos. 4,708,930 and 4,743,543); a human colorectal cancer antigen (U.S. Pat. No. 4,921,789); CA125 antigen from cystadenocarcinoma (U.S. Pat. No. 4,921,790); DF3 antigen from human breast carcinoma (U.S. Pat. Nos. 4,963,484 and 5,053,489); a human breast tumor antigen (U.S. Pat. No. 4,939,240); p97 antigen of human melanoma (U.S. Pat. No. 4,918,164); carcinoma or orosomucoid-related antigen (CORA) (U.S. Pat. No. 4,914,021); a human pulmonary carcinoma antigen that reacts with human squamous cell lung carcinoma but not with human small cell lung carcinoma (U.S. Pat. No. 4,892,935); T and Tn haptens in glycoproteins of human breast carcinoma (Springer et al., Carbohydr. Res. 178:271-292 (1988)), MSA breast carcinoma glycoprotein termed (Tjandra et al., Br. J. Surg. 75:811-817 (1988)); MFGM breast carcinoma antigen (Ishida et al., Tumor Biol. 10:12-22 (1989)); DU-PAN-2 pancreatic carcinoma antigen (Lan et al., Cancer Res. 45:305-310 (1985)); CA125 ovarian carcinoma antigen (Hanisch et al., Carbohydr. Res. 178:29-47 (1988)); YH206 lung carcinoma antigen (Hinoda et al., (1988) Cancer J. 42:653-658 (1988)). Each of the foregoing references are specifically incorporated herein by reference.

Various procedures known in the art are used for the production of polyclonal antibodies. For the production of antibody, various host animals can be immunized by injection with the peptide corresponding to the desired epitope including but not limited to rabbits, mice, rats, sheep, goats, etc. In a preferred embodiment, the peptide is conjugated to an immunogenic carrier (e.g., diphtheria toxoid, bovine serum albumin (BSA), or keyhole limpet hemocyanin (KLH)). Various adjuvants are used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum.

For preparation of monoclonal antibodies, any technique that provides for the production of antibody molecules by continuous cell lines in culture may be used (See e.g., Harlow and Lane, Antibodies: A Laboratory Manual, Cold Spring Harbor Laboratory Press, Cold Spring Harbor, N.Y.). These include, but are not limited to, the hybridoma technique originally developed by Kohler and Milstein (Kohler and Milstein, Nature 256:495-497 (1975)), as well as the trioma technique, the human B-cell hybridoma technique (See e.g., Kozbor et al. Immunol. Today 4:72 (1983)), and the EBV-hybridoma technique to produce human monoclonal antibodies (Cole et al., in Monoclonal Antibodies and Cancer Therapy, Alan R. Liss, Inc., pp. 77-96 (1985)).

In an additional embodiment of the invention, monoclonal antibodies can be produced in germn-free animals utilizing recent technology (See e.g., PCT/US90/02545). According to the invention, human antibodies may be used and can be obtained by using human hybridomas (Cote et al., Proc. Natl. Acad. Sci. U.S.A. 80:2026-2030 (1983)) or by transforming human B cells with EBV virus in vitro (Cole et al., in Monoclonal Antibodies and Cancer Therapy, Alan R. Liss, pp. 77-96 (1985)).

According to the invention, techniques described for the production of single chain antibodies (U.S. Pat. No. 4,946,778; herein incorporated by reference) can be adapted to produce specific single chain antibodies. An additional embodiment of the invention utilizes the techniques described for the construction of Fab expression libraries (Huse et al., Science 246:1275-1281 (1989)) to allow rapid and easy identification of monoclonal Fab fragments with the desired specificity.

Antibody fragments that contain the idiotype (antigen binding region) of the antibody molecule can be generated by known techniques. For example, such fragments include but are not limited to: the F(ab′)2 fragment that can be produced by pepsin digestion of the antibody molecule; the Fab′ fragments that can be generated by reducing the disulfide bridges of the F(ab′)2 fragment, and the Fab fragments that can be generated by treating the antibody molecule with papain and a reducing agent.

In the production of antibodies, screening for the desired antibody can be accomplished by techniques known in the art (e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), Western Blots, precipitation reactions, agglutination assays (e.g., gel agglutination assays, hemagglutination assays, etc.), complement fixation assays, immunofluorescence assays, protein A assays, and immunoelectrophoresis assays, etc.).

The dendrimer systems of the present invention have many advantages over liposomes, such as their greater stability, better control of their size and polydispersity, and generally lower toxicity and immunogenicity (See e.g., Duncan et al, Polymer Preprints 39:180 (1998)). Thus, in some embodiments of the present invention, anti-HER2 antibody fragments, as well as other targeting antibodies are conjugated to dendrimers, as targeting agents for the nanodevices of the present invention.

The bifunctional linkers SPDP and SMCC and the longer Mal-PEG-OSu linkers are particularly useful for antibody-dendrimer conjugation. In addition, many tumor cells contain surface lectins that bind to oligosaccharides, with specific recognition arising chiefly from the terminal carbohydrate residues of the latter (See, e.g., Sharon and L is, Science 246:227 (1989)). Attaching appropriate monosaccharides to nonglycosylated proteins such as BSA provides a conjugate that binds to tumor lectin much more tightly than the free monosaccharide (See, e.g., Monsigny et al., Biochemie 70:1633 (1988)).

Mannosylated PAMAM dendrimers bind mannoside-binding lectin up to 400 more avidly than monomeric mannosides (See, e.g., Page and Roy, Bioconjugate Chem., 8:714 (1997)). Sialylated dendrimers and other dendritic polymers bind to and inhibit a variety of sialate-binding viruses both in vitro and in vivo. By conjugating multiple monosaccharide residues (e.g., α-galactoside, for galactose-binding cells) to dendrimers, polyvalent conjugates are created with a high affinity for the corresponding type of tumor cell. The attachment reaction are easily carried out via reaction of the terminal amines with commercially-available .alpha.-galactosidyl-phenylisothiocyanate. The small size of the carbohydrates allows a high concentration to be present on the dendrimer surface.

Related to the targeting approaches described above is the “pretargeting” approach (See e.g., Goodwin and Meares, Cancer (suppl.) 80:2675 (1997)). An example of this strategy involves initial treatment of the patient with conjugates of tumor-specific monoclonal antibodies and streptavidin. Remaining soluble conjugate is removed from the bloodstream with an appropriate biotinylated clearing agent. When the tumor-localized conjugate is all that remains, a radiolabeled, biotinylated agent is introduced, which in turn localizes at the tumor sites by the strong and specific biotin-streptavidin interaction. Thus, the radioactive dose is maximized in dose proximity to the cancer cells and minimized in the rest of the body where it can harm healthy cells.

It has been shown that if streptavidin molecules bound to a polystyrene well are first treated with a biotinylated dendrimer, and then radiolabeled streptavidinis introduced, up to four of the labeled streptavidin molecules are bound per polystyrene-bound streptavidin (See, e.g., Wilbur et al., Bioconjugate Chem., 9:813 (1998)). Thus, biotinylated dendrimers may be used in the methods of the present invention, acting as a polyvalent receptor for the radiolabel in vivo, with a resulting amplification of the radioactive dosage per bound antibody conjugate. In the preferred embodiments of the present invention, one or more multiply-biotinylated module(s) on the clustered dendrimer presents a polyvalent target for radiolabeled or boronated (See, e.g., Barth et al., Cancer Investigation 14:534 (1996)) avidin or streptavidin, again resulting in an amplified dose of radiation for the tumor cells.

Dendrimers may also be used as clearing agents by, for example, partially biotinylating a dendrimer that has a polyvalent galactose or mannose surface. The conjugate-clearing agent complex would then have a very strong affinity for the corresponding hepatocyte receptors.

In other embodiments of the present invention, an enhanced permeability and retention (EPR) method is used in targeting. The enhanced permeability and retention (EPR) effect is a more “passive” way of targeting tumors (See, Duncan and Sat, Ann. Oncol., 9:39 (1998)). The EPR effect is the selective concentration of macromolecules and small particles in the tumor microenvironment, caused by the hyperpermeable vasculature and poor lymphatic drainage of tumors. The dendrimer compositions of the present invention provide ideal polymers for this application, in that they are relatively rigid, of narrow polydispersity, of controlled size and surface chemistry, and have interior “cargo” space that can carry and then release antitumor drugs. In fact, PAMAM dendrimer-platinates have been shown to accumulate in solid tumors (Pt levels about 50 times higher than those obtained with cisplatin) and have in vivo activity in solid tumor models for which cisplatin has no effect (See, e.g., Malik et al., Proc. Int'l. Symp. Control. Rel. Bioact. Mater., 24:107 (1997) and Duncan et al., Polymer Preprints 39:180 (1998)).

### VII. Synthesis and Conjugation

The present section provides a description of the synthesis and formation of the individual dendrimers comprising iron oxide NPs and further comprising one or more of the functional groups described above and the conjugation of such groups to the dendrimer (See, e.g., Examples 1, 2, and 18).

In preferred embodiments of the present invention, the preparation of PAMAM dendrimers is performed according to a typical divergent (building up the macromolecule from an initiator core) synthesis. It involves a two-step growth sequence that consists of a Michael addition of amino groups to the double bond of methyl acrylate (MA) followed by the amidation of the resulting terminal carbomethoxy, —(CO₂ CH₃) group, with ethylenediamine (EDA).

In the first step of this process, ammonia is allowed to react under an inert nitrogen atmosphere with MA (molar ratio: 1:4.25) at 47° C. for 48 hours. The resulting compound is referred to as generation=0, the star-branched PAMAM tri-ester. The next step involves reacting the tri-ester with an excess of EDA to produce the star-branched PAMAM tri-amine (G=O). This reaction is performed under an inert atmosphere (nitrogen) in methanol and requires 48 hours at 0° C. for completion. Reiteration of this Michael addition and amidation sequence produces generation=1.

Preparation of this tri-amine completes the first full cycle of the divergent synthesis of PAMAM dendrimers. Repetition of this reaction sequence results in the synthesis of larger generation (G=1-5) dendrimers (i.e., ester- and amine-terminated molecules, respectively). For example, the second iteration of this sequence produces generation 1, with an hexa-ester and hexa-amine surface, respectively. The same reactions are performed in the same way as for all subsequent generations from 1 to 9, building up layers of branch cells giving a core-shell architecture with precise molecular weights and numbers of terminal groups as shown above. Carboxylate-surfaced dendrimers can be produced by hydrolysis of ester-terminated PAMAM dendrimers, or reaction of succinic anhydride with amine-surfaced dendrimers (e.g., full generation PAMAM, POPAM or POPAM-PAMAM hybrid dendrimers).

Various dendrimers can be synthesized based on the core structure that initiates the polymerization process. These core structures dictate several important characteristics of the dendrimer molecule such as the overall shape, density, and surface functionality (See, e.g., Tomalia et al., Angew. Chem. Int. Ed. Engl., 29:5305 (1990)). Spherical dendrimers derived from ammonia possess trivalent initiator cores, whereas EDA is a tetra-valent initiator core. Recently, rod-shaped dendrimers have been reported which are based upon linear poly(ethyleneimine) cores of varying lengths the longer the core, the longer the rod (See, e.g., Yin et al., J. Am. Chem. Soc., 120:2678 (1998)).

In some embodiments, dendrimers of the present invention comprise a protected core diamine. In some embodiments, the protected initiator core diamine is NH2-(CH2)ₙ-NHPG, (n=1-10). In other embodiments, the intitor core is selected from the group comprising, but not limited to, NH2-(CH2)ₙ-NH2 (n=1-10), NH2-((CH2)ₙNH2)₃ (n=1-10), or unsubstituted or substituted 1,2-; 1,3-; or 1,4-phenylenedi-n-alkylamine, with a monoprotected diamine (e.g., NH2-(CH2)ₙ-NHPG) used during the amide formation of each generation. In these approaches, the protected diamine allows for the large scale production of dendrimers without the production of non-uniform nanostructures that can make characterization and analysis difficult. By limiting the reactivity of the diamine to only one terminus, the opportunities of dimmer/polymer formation and intramolecular reactions are obviated without the need of employing large excesses of diamine. The terminus monoprotected intermediates can be readily purified since the protecting groups provide suitable handle for productive purifications by classical techniques like crystallization and or chromatography.

The protected intermediates can be deprotected in a deprotection step, and the resulting generation of the dendrimer subjected to the next iterative chemical reaction without the need for purification. The invention is not limited to a particular protecting group. Indeed a variety of protecting groups are contemplated including, but not limited to, t-butoxycarbamate (N-t-Boc), allyloxycarbamate (N-Alloc), benzylcarbamate (N-Cbz), 9-fluorenylmethylcarbamate (FMOC), or phthalimide (Phth). In preferred embodiments of the present invention, the protecting group is benzylcarbamate (N-Cbz). N-Cbz is ideal for the present invention since it alone can be easily cleaved under “neutral” conditions by catalytic hydrogenation (Pd/C) without resorting to strongly acidic or basic conditions needed to remove an F-MOC group. The use of protected monomers finds particular use in high through-put production runs because a lower amount of monomer can be used, reducing production costs.

The dendrimers may be characterized for size and uniformity by any suitable analytical techniques. These include, but are not limited to, atomic force microscopy (AFM), electrospray-ionization mass spectroscopy, MALDI-TOF mass spectroscopy, ¹³C nuclear magnetic resonance spectroscopy, high performance liquid chromatography (HPLC) size exclusion chromatography (SEC) (equipped with multi-angle laser light scattering, dual UV and refractive index detectors), capillary electrophoresis and get electrophoresis. These analytical methods assure the uniformity of the dendrimer population and are important in the quality control of dendrimer production for eventual use in in vivo applications. Most importantly, extensive work has been performed with dendrimers showing no evidence of toxicity when administered intravenously (Roberts et al., J. Biomed. Mater. Res., 30:53 (1996) and Boume et al., J. Magnetic Resonance Imaging, 6:305 (1996)).

### VIII. Evaluation of Anti-Tumor Efficacy and Toxicity of Dendrimers

The anti-tumor effects of various therapeutic agents on cancer cell lines and primary cell cultures may be evaluated using the nanodevices of the present invention. For example, in preferred embodiments, assays are conducted, in vitro, using established tumor cell line models or primary culture cells (See, e.g., Examples 10-13).

### A. Quantifying the Induction of Apoptosis of Human Tumor Cells In Vitro

In an exemplary embodiment of the present invention, the nanodevices of the present invention are used to assay apoptosis of human tumor cells in vitro. Testing for apoptosis in the cells determines the efficacy of the therapeutic agent. Multiple aspects of apoptosis can and should be measured. These aspects include those described above, as well as aspects including, but are not limited to, measurement of phosphatidylserine (PS) translocation from the inner to outer surface of plasma membrane, measurement of DNA fragmentation, detection of apoptosis related proteins, and measurement of Caspase-3 activity.

### B. In Vitro Toxicology

In some embodiments of the present invention, to gain a general perspective into the safety of a particular nanodevice platform or component of that system, toxicity testing is performed. Toxicological information may be derived from numerous sources including, but not limited to, historical databases, in vitro testing, and in vivo animal studies.

In vitro toxicological methods have gained popularity in recent years due to increasing desires for alternatives to animal experimentation and an increased perception to the potential ethical, commercial, and scientific value. In vitro toxicity testing systems have numerous advantages including improved efficiency, reduced cost, and reduced variability between experiments. These systems also reduce animal usage, eliminate confounding systemic effects (e.g., immunity), and control environmental conditions.

Although any in vitro testing system may be used with the present invention, the most common approach utilized for in vitro examination is the use of cultured cell models. These systems include freshly isolated cells, primary cells, or transformed cell cultures. Cell culture as the primary means of studying in vitro toxicology is advantageous due to rapid screening of multiple cultures, usefulness in identifying and assessing toxic effects at the cellular, subcellular, or molecular level. In vitro cell culture methods commonly indicate basic cellular toxicity through measurement of membrane integrity, metabolic activities, and subcellular perturbations. Commonly used indicators for membrane integrity include cell viability (cell count), clonal expansion tests, trypan blue exclusion, intracellular enzyme release (e.g. lactate dehydrogenase), membrane permeability of small ions (K¹, Ca²⁺), and intracellular Ala accumulation of small molecules (e.g., ⁵¹Cr, succinate). Subcellular perturbations include monitoring mitochondrial enzyme activity levels via, for example, the MTT test, determining cellular adenine triphosphate (ATP) levels, neutral red uptake into lysosomes, and quantification of total protein synthesis. Metabolic activity indicators include glutathione content, lipid peroxiidation, and lactate/pyruvate ratio.

### C. MTT Assay

The MTT assay is a fast, accurate, and reliable methodology for obtaining cell viability measurements. The MTT assay was first developed by Mosmann (Mosmann, J. Immunol. Meth., 65:55 (1983)). It is a simple colorimetric assay numerous laboratories have utilized for obtaining toxicity results (See e.g., Kuhlmann et al., Arch. Toxicol., 72:536 (1998)). Briefly, the mitochondria produce ATP to provide sufficient energy for the cell. In order to do this, the mitochondria metabolize pyruvate to produce acetyl CoA. Within the mitochondria, acetyl CoA reacts with various enzymes in the tricarboxylic acid cycle resulting in subsequent production of ATP. One of the enzymes particularly useful in the MTT assay is succinate dehydrogenase. MTT (3-(4,5-dimethylthiazol-2-yl)-2 diphenyl tetrazolium bromide) is a yellow substrate that is cleaved by succinate dehydrogenase forming a purple formazan product. The alteration in pigment identifies changes in mitochondria function. Nonviable cells are unable to produce formazan, and therefore, the amount produced directly correlates to the quantity of viable cells. Absorbance at 540 nm is utilized to measure the amount of formazan product.

The results of the in vitro tests can be compared to in vivo toxicity tests in order to extrapolate to live animal conditions. Typically, acute toxicity from a single dose of the substance is assessed. Animals are monitored over 14 days for any signs of toxicity (increased temperature, breathing difficulty, death, etc). Traditionally, the standard of acute toxicity is the median lethal dose (LD₅₀), which is the predicted dose at which half of the treated population would be killed. The determination of this dose occurs by exposing test animals to a geometric series of doses under controlled conditions. Other tests include subacute toxicity testing, which measures the animal's response to repeated doses of the nanodevice for no longer than 14 days. Subchronic toxicity testing involves testing of a repeated dose for 90 days. Chronic toxicity testing is similar to subchronic testing but may last for over a 90-day period. In vivo testing can also be conducted to determine toxicity with respect to certain tissues. For example, in some embodiments of the present invention tumor toxicity (i.e., effect of the compositions of the present invention on the survival of tumor tissue) is determined (e.g., by detecting changes in the size and/or growth of tumor tissues).

### IX. Gene Therapy Vectors

In particular embodiments of the present invention, the dendrimer compositions comprise transgenes for delivery and expression to a target cell or tissue, in vitro, ex vivo, or in vivo. In such embodiments, rather than containing the actual protein, the dendrimer complex comprises an expression vector construct containing, for example, a heterologous DNA encoding a gene of interest and the various regulatory elements that facilitate the production of the particular protein of interest in the target cells.

In some embodiments, the gene is a therapeutic gene that is used, for example, to treat cancer, to replace a defective gene, or a marker or reporter gene that is used for selection or monitoring purposes. In the context of a gene therapy vector, the gene may be a heterologous piece of DNA. The heterologous DNA may be derived from more than one source (i.e., a multigene construct or a fusion protein). Further, the heterologous DNA may include a regulatory sequence derived from one source and the gene derived from a different source.

Tissue-specific promoters may be used to effect transcription in specific tissues or cells so as to reduce potential toxicity or undesirable effects to non-targeted tissues. For example, promoters such as the PSA, probasin, prostatic acid phosphatase or prostate-specific glandular kallikrein (hK2) may be used to target gene expression in the prostate. Similarly, promoters may be used to target gene expression in other tissues (e.g., insulin, elastin amylase, pdr-1, pdx-1 and glucokinase promoters target to the pancreas; albumin PEPCK, HBV enhancer, alpha fetoproteinapolipoprotein C, alpha-1 antitrypsin, vitellogenin, NF-AB and transthyretin promoters target to the liver; myosin H chain, muscle creatine kinase, dystrophin, calpain p94, skeletal alpha-actin, fast troponin 1 promoters target to skeletal muscle; keratin promoters target the skin; sm22 alpha; SM-.alpha.-actin promoters target smooth muscle; CFTR; human cytokeratin 18 (K₁₈); pulmonary surfactant proteins A, B and Q CC-10; P1 promoters target lung tissue; endothelin-1; E-selectin; von Willebrand factor; KDR/flk-1 target the endothelium; tyrosinase targets melanocytes).

The nucleic acid may be either cDNA or genomic DNA. The nucleic acid can encode any suitable therapeutic protein. Preferably, the nucleic acid encodes a tumor suppressor, cytokine, receptor, inducer of apoptosis, or differentiating agent. The nucleic acid may be an antisense nucleic acid. In such embodiments, the antisense nucleic acid may be incorporated into the nanodevice of the present invention outside of the context of an expression vector.

In preferred embodiments, the nucleic acid encodes a tumor suppressor, cytokines, receptors, or inducers of apoptosis. Suitable tumor suppressors include BRCA1, BRCA2, C-CAM, p16, p211 p53, p73, or Rb. Suitable cytokines include GMCSF, IL-1, IL-2, IL-3, IL-4, IL-5, IL6, IL-7, IL-8, IL-9, IL-10, IL-11, IL-12, IL-13, IL-14, IL-15, β-interferon, γ-interferon, or TNF. Suitable receptors include CFTR, EGFR, estrogen receptor, IL-2 receptor, or VEGFR. Suitable inducers of apoptosis include AdE1B, Bad, Bak, Bax, Bid, Bik, Bim, Harakiri, or ICE-CED3 protease.

### X. Methods of Combined Therapy

Tumor cell resistance to DNA damaging agents represents a major problem in clinical oncology. The nanodevices of the present invention provide means of ameliorating this problem by effectively administering a combined therapy approach. However, it should be noted that traditional combination therapy may be employed in combination with the nanodevices of the present invention. For example, in some embodiments of the present invention, nanodevices may be used before, after, or in combination with the traditional therapies.

To kill cells, inhibit cell growth, or metastasis, or angiogenesis, or otherwise reverse or reduce the malignant phenotype of tumor cells using the methods and compositions of the present invention in combination therapy, one contacts a “target” cell with the nanodevices compositions described herein and at least one other agent. These compositions are provided in a combined amount effective to kill or inhibit proliferation of the cell. This process may involve contacting the cells with the immunotherapeutic agent and the agent(s) or factor(s) at the same time. This may be achieved by contacting the cell with a single composition or pharmacological formulation that includes both agents, or by contacting the cell with two distinct compositions or formulations, at the same time, wherein one composition includes, for example, an expression construct and the other includes a therapeutic agent.

Alternatively, the nanodevice treatment may precede or follow the other agent treatment by intervals ranging from minutes to weeks. In embodiments where the other agent and immunotherapy are applied separately to the cell, one would generally ensure that a significant period of time did not expire between the time of each delivery, such that the agent and nanodevice would still be able to exert an advantageously combined effect on the cell. In such instances, it is contemplated that cells are contacted with both modalities within about 12-24 hours of each other and, more preferably, within about 6-12 hours of each other, with a delay time of only about 12 hours being most preferred. In some situations, it may be desirable to extend the time period for treatment significantly, however, where several days (2 to 7) to several weeks (1 to 8) lapse between the respective administrations.

In some embodiments, more than one administration of the immunotherapeutic composition of the present invention or the other agent are utilized. Various combinations may be employed, where the dendrimer is “A” and the other agent is “B”, as exemplified below:

A/B/A, B/A/B, B/B/A, A/A/B, B/A/A, A/B/B, B/B/B/A, B/B/A/B,
A/A/B/B, A/B/A/B, A/B/B/A, B/B/A/A, B/A/B/A, B/A/A/B, B/B/B/A,
A/A/A/B, B/A/A/A, A/B/A/A, A/A/B/A, A/B/B/B, B/A/B/B, B/B/A/B.

Other combinations are contemplated. Again, to achieve cell killing, both agents are delivered to a cell in a combined amount effective to kill or disable the cell.

Other factors that may be used in combination therapy with the nanodevices of the present invention include, but are not limited to, factors that cause DNA damage such as .gamma.-rays, X-rays, and/or the directed delivery of radioisotopes to tumor cells. Other forms of DNA damaging factors are also contemplated such as microwaves and UV-irradiation. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells. The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

In preferred embodiments of the present invention, the regional delivery of the nanodevice to patients with cancers is utilized to maximize the therapeutic effectiveness of the delivered agent. Similarly, the chemo- or radiotherapy may be directed to particular, affected region of the subjects body. Alternatively, systemic delivery of the immunotherapeutic composition and/or the agent may be appropriate in certain circumstances, for example, where extensive metastasis has occurred.

In addition to combining the nanodevice with chemo- and radiotherapies, it also is contemplated that traditional gene therapies are used. For example, targeting of p53 or p16 mutations along with treatment of the nanodevices provides an improved anti-cancer treatment. The present invention contemplates the co-treatment with other tumor-related genes including, but not limited to, p21, Rb, APC, DCC, NF-I, NF-2, BCRA2, p16, FHIT, WT-I, MEN-I, MEN-II, BRCA1, VHL, FCC, MCC, ras, myc, neu, raf erb, src, fms, jun, trk, ret, gsp, hst, bcl, and abl.

In vivo and ex vivo treatments are applied using the appropriate methods worked out for the gene delivery of a particular construct for a particular subject. For example, for viral vectors, one typically delivers 1×10⁴, 1×10⁵, 1×10⁶, 1×10⁷, 1×10⁸, 1×10⁹, 1×10¹⁰, 1×10¹¹ or 1×10¹² infectious particles to the patient. Similar figures may be extrapolated for liposomal or other non-viral formulations by comparing relative uptake efficiencies.

An attractive feature of the present invention is that the therapeutic compositions may be delivered to local sites in a patient by a medical device. Medical devices that are suitable for use in the present invention include known devices for the localized delivery of therapeutic agents. Such devices include, but are not limited to, catheters such as injection catheters, balloon catheters, double balloon catheters, microporous balloon catheters, channel balloon catheters, infusion catheters, perfusion catheters, etc., which are, for example, coated with the therapeutic agents or through which the agents are administered; needle injection devices such as hypodermic needles and needle injection catheters; needleless injection devices such as jet injectors; coated stents, bifurcated stents, vascular grafts, stent grafts, etc.; and coated vaso-occlusive devices such as wire coils.

Exemplary devices are described in U.S. Pat. Nos. 5,935,114; 5,908,413; 5,792,105; 5,693,014; 5,674,192; 5,876,445; 5,913,894; 5,868,719; 5,851,228; 5,843,089; 5,800,519; 5,800,508; 5,800,391; 5,354,308; 5,755,722; 5,733,303; 5,866,561; 5,857,998; 5,843,003; and 5,933,145; the entire contents of which are incorporated herein by reference. Exemplary stents that are commercially available and may be used in the present application include the RADIUS (Scimed Life Systems, Inc.), the SYMPHONY (Boston Scientific Corporation), the Wallstent (Schneider Inc.), the PRECEDENT II (Boston Scientific Corporation) and the NIR (Medinol Inc.). Such devices are delivered to and/or implanted at target locations within the body by known techniques.

### XI. Photodynamic Therapy

In some embodiments, the therapeutic complexes of the present invention comprise a photodynamic compound and a targeting agent that is administred to a patient. In some embodiments, the targeting agent is then allowed a period of time to bind the “target” cell (e.g. about 1 minute to 24 hours) resulting in the formation of a target cell-target agent complex. In some embodiments, the therapeutic complexes comprising the targeting agent and photodynamic compound are then illuminated (e.g., with a red laser, incandescent lamp, X-rays, or filtered sunlight). In some embodiments, the light is aimed at the jugular vein or some other superficial blood or lymphatic vessel. In some embodiments, the singlet oxygen and free radicals diffuse from the photodynamic compound to the target cell (e.g. cancer cell or pathogen) causing its destruction.

### XII. Pharmaceutical Formulations

Where clinical applications are contemplated, in some embodiments of the present invention, the nanodevices are prepared as part of a pharmaceutical composition in a form appropriate for the intended application. Generally, this entails preparing compositions that are essentially free of pyrogens, as well as other impurities that could be harmful to humans or animals. However, in some embodiments of the present invention, a straight dendrimer formulation may be administered using one or more of the routes described herein.

In preferred embodiments, the dendrimers are used in conjunction with appropriate salts and buffers to render delivery of the compositions in a stable manner to allow for uptake by target cells. Buffers also are employed when the nanodevices are introduced into a patient. Aqueous compositions comprise an effective amount of the nanodevice to cells dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. Except insofar as any conventional media or agent is incompatible with the vectors or cells of the present invention, its use in therapeutic compositions is contemplated. Supplementary active ingredients may also be incorporated into the compositions.

In some embodiments of the present invention, the active compositions include classic pharmaceutical preparations. Administration of these compositions according to the present invention is via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection.

The active nanodevices may also be administered parenterally or intraperitoneally or intratumorally. Solutions of the active compounds as free base or pharmacologically acceptable salts are prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

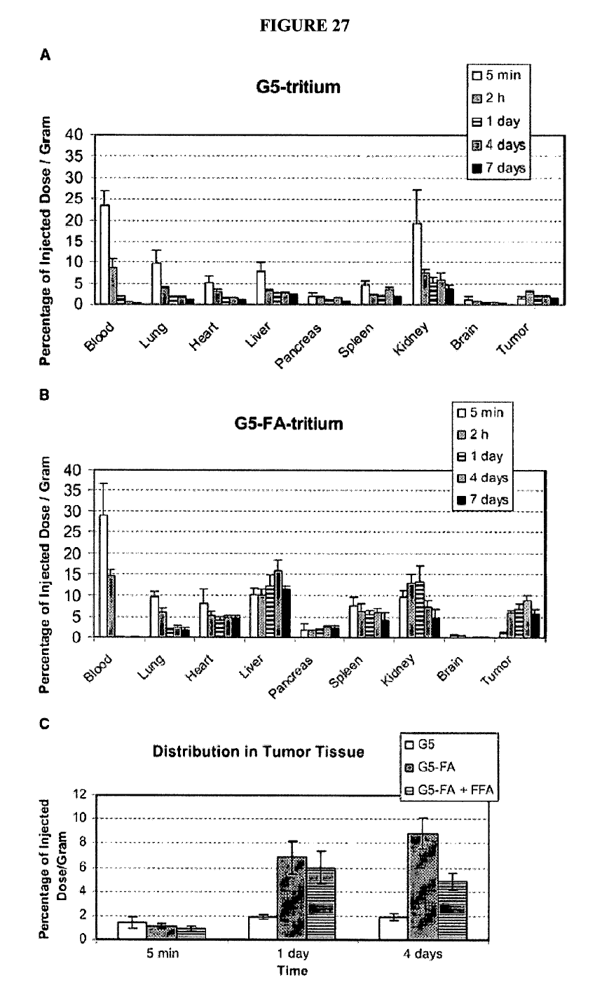

In some embodiments, the present invention provides a composition comprising a dendrimer comprising a targeting agent, a therapeutic agent and an imaging agent. In some embodiments, the dendrimer is used for delivery of a therapeutic agent (e.g., methotrexate) to tumor cells in vivo (See, e.g., Example 13, FIG. 27). In some embodiments, the therapeutic agent is conjugated to the dendrimer via an acid-labile linker. Thus, in some embodiments, the therapeutic agent is released from the dendrimer within a target cell (e.g., within an endosome). This type of intracellular release (e.g., endosomal disruption of the acid-labile linker) is contemplated to provide additional specificity for the compositions and methods of the present invention. In preferred embodiments, the dendrimers of the present invention (e.g., G5 PAMAM dendrimers) contain between 100-150 primary amines on the surface (See, e.g., Example 13). Thus, the present invention provides dendrimers with multiple (e.g., 100-150) reactive sites for the conjugation of functional groups comprising, but not limited to, therapeutic agents, targeting agents, imaging agents and biological monitoring agents.

The compositions and methods of the present invention are contemplated to be equally effective whether or not the dendrimer compositions of the present invention comprise a fluorescein (e.g. FITC) imaging agent (See, e.g., Example 13). Thus, each functional group present in a dendrimer composition is able to work independently of the other functional groups. Thus, the present invention provides a dendrimer that can comprise multiple combinations of targeting, therapeutic, imaging, and biological monitoring functional groups.

The present invention also provides a very effective and specific method of delivering molecules (e.g., therapeutic and imaging functional groups) to the interior of target cells (e.g., cancer cells). Thus, in some embodiments, the present invention provides methods of therapy that comprise or require delivery of molecules into a cell in order to function (e.g., delivery of genetic material such as siRNAs).

The pharmaceutical forms suitable for injectable use include sterile aqueous solutions or dispersions and sterile powders for the extemporaneous preparation of sterile injectable solutions or dispersions. The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it may be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, the dendrimer compositions are administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution is suitably buffered, if necessary, and the liquid diluent first rendered isotonic with sufficient saline or glucose. These particular aqueous solutions are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). In some embodiments of the present invention, the active particles or agents are formulated within a therapeutic mixture to comprise about 0.0001 to 1.0 milligrams, or about 0.001 to 0.1 milligrams, or about 0.1 to 1.0 or even about 10 milligrams per dose or so. Multiple doses may be administered.

Additional formulations that are suitable for other modes of administration include vaginal suppositories and pessaries. A rectal pessary or suppository may also be used. Suppositories are solid dosage forms of various weights and shapes, usually medicated, for insertion into the rectum, vagina or the urethra. After insertion, suppositories soften, melt or dissolve in the cavity fluids. In general, for suppositories, traditional binders and carriers may include, for example, polyalkylene glycols or triglycerides; such suppositories may be formed from mixtures containing the active ingredient in the range of 0.5% to 10%, preferably 1%-2%. Vaginal suppositories or pessaries are usually globular or oviform and weighing about 5 g each. Vaginal medications are available in a variety of physical forms, e.g., creams, gels or liquids, which depart from the classical concept of suppositories. In addition, suppositories may be used in connection with colon cancer. The nanodevices also may be formulated as inhalants for the treatment of lung cancer and such like.

### XIII. Method of Treatment or Prevention of Cancer and Pathogenic Diseases

In specific embodiments of the present invention methods and compositions are provided for the treatment of tumors in cancer therapy (See, e.g., Example 13). It is contemplated that the present therapy can be employed in the treatment of any cancer for which a specific signature has been identified or which can be targeted. Cell proliferative disorders, or cancers, contemplated to be treatable with the methods of the present invention include human sarcomas and carcinomas, including, but not limited to, fibrosarcoma, myxosarcoma, liposarcoma, chondrosarcoma, osteogenic sarcoma, chordoma, angiosarcoma, endotheliosarcoma, lymphangiosarcoma, Ewing's tumor, lymphangioendotheliosarcoma, synovioma, mesothelioma, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilns' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrbm's macroglobulinemia, and heavy chain disease.

It is contemplated that the present therapy can be employed in the treatment of any pathogenic disease for which a specific signature has been identified or which can be targeted for a given pathogen. Examples of pathogens contemplated to be treatable with the methods of the present invention include, but are not limited to, Legionella peomophilia, Mycobacterium tuberculosis, Clostridium tetani, Hemophilus influenzae, Neisseria gonorrhoeae, Treponema pallidum, Bacillus anthracis, Vibrio cholerae, Borrelia burgdorferi, Cornebacterium diphtheria, Staphylococcus aureus, human papilloma virus, human immunodeficiency virus, rubella virus, polio virus, and the like.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: g (grams); l or L (liters); μg (micrograms); μl (microliters); μm (micrometers); μM (micromolar); μmol (micromoles); mg (milligrams); ml (milliliters); mm (millimeters); mM (millimolar); mmol (millimoles); M (molar); mol (moles); ng (nanograms); nm (nanometers); nmol (nanomoles); N (normal); pmol (picomoles); Aldrich (Sigma/Aldrich, Milwaukee, Wis.); Sigma (Sigma Chemical Co., St. Louis, Mo.); Fisher Scientific (Fisher Scientific, Pittsburgh, Pa.); Millipore (Millipore, Billerica, Mass.); Mettler Toledo Mettler Toledo (Columbus, Ohio); Waters (Waters Corporation, Milford, Mass.); Wyatt Technology (Wyatt Technology Corp., Santa Barbara, Calif.); TosoHaas (TosoHaas Corp., Montgomeryville, Pa.); Perkin Elmer (Perkin Elmer, Wellesley, Mass.); Beckman Coulter (Beckman Coulter Corp., Fullerton, Calif.); Phenomenex (Phenomenex, Torrance, Calif.); GiboBRL (GibcoBRL/Life Technologies, Gaithersburg, Md.); Pierce (Pierce Chemical Company, Rockford, Ill.); Roche (F. Hoffmann-La Roche Ltd, Basel, Switzerland).

### Example 1

### Materials and Methods

The G5 PAMAM dendrimer was synthesized and characterized at the Center for Biologic Nanotechnology, University of Michigan. MeOH(HPLC grade), acetic anhydride (99%), triethylamine (99.5%), DMSO (99.9%), fluorescein isothiocyanate (98%), glycidol (racemic form, 96%), DMF (99.8%), 1-(3-(Dimethylamino)-propyl)-3-ethylcarbodiimide HCl (EDC, 98%), citric acid (99.5%), sodium azide (99.99%), D₂O, NaCl, and volumetric solutions (0.1M HCl and 0.1M NaOH) for potentiometric titration were all purchased from Aldrich and used as received. Methotrexate (99+%) and Folic Acid (98%) were from Sigma, Spectra/Por® dialysis membrane (MWCO 3,500), Millipor Centricon ultrafiltration membrane YM-10, and phosphate buffer saline (PBS, pH=7.4) were from Fisher Scientific.

Potentiometric Titration. Titration was carried out manually using a Mettler Toledo MP230 pH Meter and MicroComb pH electrode at room temperature, 23±1° C. A 10 mL solution of 0.1 M NaCl was added to precisely weighed 100 mg of PAMAM dendrimer to shield amine group interactions. Titration was performed with 0.1028 N HCl, and 0.1009 N NaOH was used for back titration. The numbers of primary and tertiary amines were determined from back titration data.

Gel Permeation Chromatography (GPC). GPC experiments were performed on an Alliance Waters 2690 Separation Module equipped with 2487 Dual Wavelength UV Absorbance Detector (Waters), a Wyatt Dawn® DSP Laser Photometer, an Optilab DSP Interferometric Refractometer (Wyatt Technology), and with TosoHaas TSK-Gel® Guard PHW 06762 (75×7.5 mm, 12 μm), G 2000 PW 05761 (300×7.5 mm, 10 μm), G 3000 PW 05762 (300×7.5 mm, 10 μm), and G 4000 PW (300×7.5 mm, 17 μm) columns. Column temperature was maintained at 25±0.1° C. by a Waters Temperature Control Module. The isocratic mobile phase was 0.1 M citric acid and 0.025 w % sodium azide, pH 2.74, at a flow rate of 1 ml/min. Sample concentration was 10 mg/5 ml with an injection volume of 100 μL. Molecular weight, and molecular weight distribution of the PAMAM dendrimer and its conjugates were determined using Astra 4.7 software (Wyatt Technology).

Nuclear Magnetic Resonance Spectroscopy: ¹H and ¹³C NMR spectra were taken in D₂O and were used to provide integration values for structural analysis by means of a Bruker AVANCE DRX 500 instrument.

UV Spectrophotometry. UV spectra were recorded using Perkin Elmer UV/VIS

Spectrometer Lambda 20 and Lambda 20 software, in PBS.

Reverse Phase High Performance Liquid Chromatography. A reverse phase ion-pairing high performance liquid chromatography (RP-HPLC) system consisted of a System GOLD™ 126 solvent module, a Model 507 auto sampler equipped with a 100 μl loop, and a Model 166 UV detector (Beckman Coulter). A Phenomenex Jupiter C5 silica based HPLC column (250×4.6 mm, 300 Å) was used for separation of analytes. Two Phenomenex Widepore C5 guard columns (4×3 mm) were also installed upstream of the HPLC column. The mobile phase for elution of PAMAM dendrimers was a linear gradient beginning with 90:10 water/acetonitrile (ACN) at a flow rate of 1 ml/min, reaching 50:50 after 30 minutes. Trifluoroacetic acid (TFA) at 0.14 w % concentration in water as well as in ACN was used as counter-ion to make the dendrimer-conjugate surfaces hydrophobic. The conjugates were dissolved in the mobile phase (90:10 water/ACN). The injection volume in each case was 50 μl with a sample concentration of approximately 1 mg/ml and the detection of eluted samples was performed at 210, or 242, or 280 nm. The analysis was performed using Beckman's System GOLD™ Nouveau software. Characterization of each device and all intermediates has been performed through the use of UV, HPLC, NMR, and GPC.

The KB cells were obtained from ATCC (CLL17; Rockville, Md.). Trypsin-EDTA, Dulbecco's phosphate-buffered saline (PBS), fetal bovine serum, cell culture antibiotics and RPMI medium were obtained from Gibco/BRL. All other reagents were from Sigma. The synthesis and characterization of the dendrimer-conjugates is reported as a separate communication. All the dendrimer preparations used in this study were synthesized at our center and have been surface neutralized by acetylation of the free surface amino groups.

Cell culture and treatment. KB cells were maintained in folate-free medium containing 10% serum (See, e.g., Quintana et al, Pharm. Res. 19, 1310 (2002)) to provide extracellular FA similar to that found in human serum. Cells were plated in 12-well plates for uptake studies, in 24-well plates for cell growth analysis, and in 96-well plates for XTT assay. Cells were rinsed with FA-free medium containing dialyzed serum and incubated at 37° C. with dendrimer-drug conjugates for the indicated time periods and concentrations. KB cells were also maintained in RPMI medium containing 2 μM FA to obtain cells which express low FAR.

Flow Cytometry and Confocal Microscopy. The standard fluorescence of the dendrimer solutions was quantified using a Beckman spectrofluorimeter. For flow cytometric analysis of the uptake of the targeted polymer, cells were trypsinized and suspended in PBS containing 0.1% bovine serum albumin (PBSB) and analyzed using a Becton Dickinson FACScan analyzer. The FL1-fluorescence of 10,000 cells was measured and the mean fluorescence of gated viable cells was quantified. Confocal microscopic analysis was performed in cells plated on a glass cover-slip, using a Carl Ziess confocal microscope. Fluorescence and differential interference contrast (DIC) images were collected simultaneously using an argon laser, using the appropriate filters for FITC.

Evaluation of dendrimer cytotoxicity. Cell growth was determined by assay of the total protein in lysates of treated cells using a bicinchoninic acid reagent (PIERCE, and by XTT assay, using a kit from Roche.

### Example 2

### Syntheses of Dendrimer

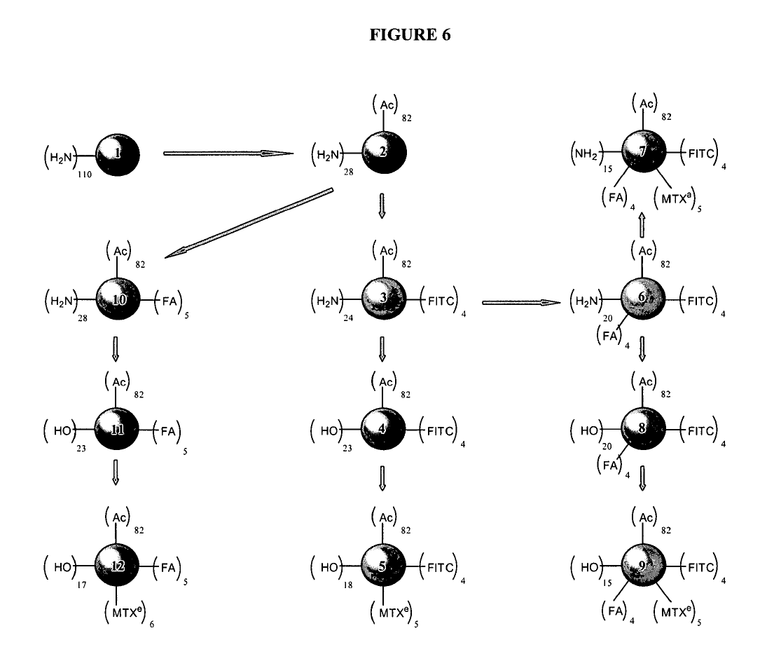

Dendrimers were synthesized according to the following process (See, e.g., FIG. 6):

```
  1. G5 carrier
  2. G5-Ac³(82)
  3. G5-Ac³(82)-FITC
  4. G5-Ac³(82)-FITC-OH
  5. G5-Ac³(82)-FITC-OH-MTX^{e}
  6. G5-Ac³(82)-FITC-FA
  7. G5-Ac¹(82)-FITC-FA-MTX^{a}
  8. G5-Ac³(82)-FITC-FA-OH
  9. G5-AC³(82)-FITC-FA-OH-MTX^{e}
10. G5-Ac²(82)-FA
11. G5-Ac²(82)-FA-OH
12. G5-Ac²(82)-FA-OH-MTX^{e}
(Note: The superscripts indicated in Ac¹, Ac², Ac³are utilized to differentiate different sets of acetylation reactions).
```

1. G5 carrier. The PAMAM G5 dendrimer was synthesized and characterized at the Center for Biologic Nanotechnology, University of Michigan. PAMAM dendrimers are composed of an ethylenediamine (EDA) initiator core with four radiating dendron arms, and are synthesized using repetitive reaction sequences comprised of exhaustive Michael addition of methyl acrylate (MA) and condensation (amidation) of the resulting ester with large excesses of EDA to produce each successive generation. Each successive reaction therefore theoretically doubles the number of surface amino groups, which can be activated for functionalization. The synthesized dendrimer has been analyzed and the molecular weight has been found to be 26,380 g/mol by GPC and the average number of primary amino groups has been determined by potentiometric titration to be 110.

2. G5-Ac³(82). 2.38696 g (8.997*10⁻⁵ Mol) of G5 PAMAM dendrimer (MW=26,380 g/mol by GPC, number of primary amines=110 by potentiometric titration) in 160 ml of abs. MeOH was allowed to react with 679.1 μl (7.198*10⁻³ mol) of acetic anhydride in the presence of 1.254 ml (8.997*10⁻³ mol, 25% molar excess) triethylamine. After intensive dialysis and lyophilization 2.51147 g (93.4%) G5-Ac³(82) product was yielded. The average number of acetyl groups (82) has been determined based on ¹H NMR calibration (Majoros, I. J., Keszler, B., Woehler, S., Bull, T., and Baker, J. R., Jr. (2003)).

3. G5-Ac³(82) —FITC. 1.16106 g (3.884*10⁻⁵ mol) of G5-Ac³(82) partially acetylated PAMAM (MW=29,880 g/mol by GPC) in 110 ml of abs. DMSO was allowed to react with 0.08394 g (90% pure) (1.94*10⁻⁴ mol) of FITC under nitrogen overnight. After intensive dialysis, lyophilization 1.10781 g, (89.58%) G5-Ac³(82) —FITC product was yielded. Further purification was done through membrane filtration.

4. G5-Ac³(82) —FITC-OH. 0.20882 g (6.51*10⁻⁶ mol) of G5-Ac³(82) —FITC was allowed to react with 19.9 μl (2.99*10⁻⁴ mol) of glycidol (racemic) in 150 ml of DI water. Two glycidol molecules were calculated for each remaining primary amino group. The reaction mixture was stirred vigorously for 3 hrs at room temperature. After intensive dialysis for 2 days, and lyophilization, the yield of the product G5-Ac³(82) —FITC-OH was 0.18666 g (84.85%).

5. G5-Ac³(82) —FITC-OH-MTXe. 0.02354 g MTX (5.18*10⁻⁵ mol) was allowed to react with 0.13269 g (6.92*10⁻⁴ mol) EDC in 27 ml DMF and 9 ml DMSO for 1 hr at room temperature with vigorous stirring. This solution was added drop wise to 150 ml DI water solution containing 0.09112 g (2.72*10⁻⁶ mol) of G5-Ac³(82)-FITC-OH. The reaction was vigorously stirred for 3 days at room temperature. After intense dialysis, and lyophilization, the yield of the targeted molecule G5-Ac³(82) —FITC-OH-MTXe was 0.08268 g (73.5%).

6. G5-Ac³(82) —FITC-FA. 0.03756 g (8.51*10⁻⁵ mol) of FA (MW=441.4 g/mol) was allowed to react with 0.23394 g (1.22*10⁻³ mol) of EDC (1-(3-(dimethylamino)-propyl)-3-ethylcarbodiimide HCl; MW=191.71 g/mol) in 27 ml dry DMF and 9 ml dry DMSO mixture under nitrogen atmosphere for 1 hr. Then this organic reaction mixture was added drop wise to the DI water solution (100 ml) of 0.49597 g (1.55*10⁻⁵ mol; MW=32,150 g/mol) G5-Ac³(82) —FITC. The reaction mixture was vigorously stirred for 2 days. After dialysis and lyophilization G5-Ac³(82) —FITC-FA weight was 0.5202 g (98.1%). Further purification was done through membrane filtration.

7. G5-Ac¹(82)-FITC-FA-MTXa. 2.1763*10⁻⁵ mol (0.00989 g) of MTX (MW=454.45 g/mol) was allowed to react with 3.0948*10⁻⁴ mol (0.05933 g) of EDC in 66 ml dry DMF and 22 ml dry DMSO mixture under nitrogen atmosphere for 1 hr. This organic reaction mixture was added drop wise to the DI water solution (260 ml) of 0.09254 g (2.7051*10⁻⁶ mol; MW=34,710 g/mol by GPC) G5-Ac¹(82)-FITC-FA-NH₂. The solution was vigorously stirred for 2 days. After dialysis and lyophilization G5-Ac¹(82)-FITC-FA-MTX^{a} weight was 0.09503 g (96.5%). Further purification was done by membrane filtration before analysis. This three-functional device served as a control compound in drug cleavage in in vitro cytotoxicity study.

8. G5-Ac³(82) —FITC-FA-OH. 0.29597 g (8.63*10⁻⁶ mol) of G5-Ac³(82) —FITC-FA partially acetylated PAMAM dendrimer conjugate (MW=34,710 g/mol by GPC) in 200 ml of DI water was allowed to react with 20.6 μl (3.1*10⁻⁴ mol, 25% molar excess) of glycidol (MW=74.08 g/mol) for 3 hrs. After intensive dialysis, lyophilization and repeated membrane filtration 0.27787 g (90.35%) fully glycidylated G5-Ac³(82) —FITC-FA-OH product was yielded. Non-specific uptake was not observed in in vitro study (see Part II of this research for uptake study (24).

9. G5-Ac³(82) —FITC-FA-OH-MTXe. 0.03848 g (8.4674*10⁻⁵ mol) of MTX (MW=454.45 g/mol) was allowed to react with 0.22547 g (1.176*10⁻³ mol) of EDC in 54 ml dry DMF and 18 ml dry DMSO mixture under nitrogen atmosphere for 1 hr. This organic reaction mixture was added drop wise to the DI water solution (240 ml) of 0.16393 g (4.6339*10⁻⁶ mol; MW=36,820 g/mol) G5-Ac³(82) —FITC-FA-OH. The solution was vigorously stirred for 3 days. After dialysis, repeated membrane filtration and lyophilization G5-Ac³(82) —FITC-FA-MTX^{e} weight was 0.18205 g (90.88%).

10. G5-Ac²(82) —FA. FA was attached to G5-Ac²(82) in two consecutive reactions. 0.03278 g (7.426*10⁻⁵ mol) FA was allowed to react with a 14-fold excess of EDC 0.19979 g (1.042*10⁻³ mol) in a 24 ml DMF, 8 ml DMSO solvent mixture at room temperature, then this FA-active ester solution was added drop wise to an aqueous solution of the partially acetylated product G5-Ac²(82) (0.40366 g, 1.347*10⁻⁵ mol) in 90 ml water. After dialysis and lyophilization, the product weight was 0.41791 g (96.7%). The number of FA molecules was determined by UV spectroscopy. As an additional characterization, no free FA was observed by a GPC equipped with a UV detector, or by agarose gel.

11. G5-Ac²(82) —FA-OH. 0.21174 g (6.60*10⁻⁶ mol) of mono-functional dendritic device, G5-Ac²(82) —FA, was allowed to react with 20.1 μl (3.04*10⁻⁴ mol) of glycidol in 154 ml DI water under vigorous stirring for 3 hrs. After dialysis and lyophilization the glycidolated mono-functional device, having hydroxyl groups on the surface (yield: 0.20302 g, 91.05%), participated in the conjugation reaction with methotrexate.

12. G5-Ac²(82) —FA-OH-MTX^{e}. In 27 ml of DMF and 9 ml of DMSO solvent mixture, 0.02459 g (5.41*10⁻⁵ mol) of MTX and 0.14315 g (7.46*10⁻⁴ mol) of 1-(3-(dimethylamino)-propyl)-3-ethylcarbodiimide hydrochloride (EDC) was allowed to react under nitrogen at room temperature for 1 hr. The reaction mixture was vigorously stirred. The MTX-active ester solution was added drop wise to the 0.09975 g (2.95*10⁻⁶ mol) of mono-functional dendritic device, having hydroxyl groups on the surface, in 150 ml DI water, and this reaction mixture was stirred at room temperature for 3 days. After dialysis and lyophilization this bi-functional device G5-Ac²(82) —FA-OH-MTXe (yield: 0.11544 g, 93.9%) was tested by compositional and biological matter.

### Example 3

### Potentiometric Titration Curves to Analyze Terminal Primary Amino Groups of G5 PAPAM Dendrimer

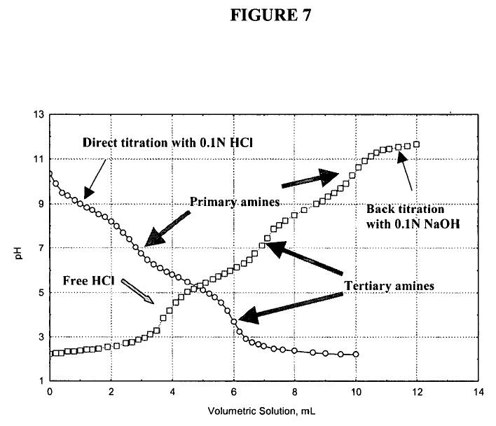

Potentiometric titration was performed to determine the number of primary and tertiary amino groups. Theoretically, the G5 PAMAM dendrimer has 128 primary amine groups on its surface, and 126 tertiary amine groups. These values can be determined through use of mathematical models. Potentiometric titration revealed that there were 110 primary amines present on the surface of the G5 PAMAM dendrimer carrier (See, e.g., FIG. 7, which shows the titration curves performed by direct titration with 0.1 M HCl volumetric solution and back-titration with 0.1 M NaOH volumetric solution). The average number of primary amino groups was calculated using back titration data performed with 0.1 M NaOH volumetric solution.

The determination of molecular weight of each conjugate structure was also necessary in order to produce a well-defined multi-functional dendrimer. GPC equipped with multi-angle laser light scattering and an RI detector as a concentration detector was used for this purpose (See, e.g., Table 1, which presents PAMAM dendrimer carrier and its mono-, bi-, and tri-functional conjugates with molecular weights and molecular weight distribution given for each. The superscript numerals 2 and 3 (ex.—G5-Ac2 and G5-Ac³) indicate that these are two independent acetylation reactions).

TABLE 1

| | M̅ₙ̅, g/mol | M̅_̅{̅w̅}̅, g/mol | M̅_̅{̅w̅}̅/ M̅ₙ̅ |
|---|---|---|---|
| G5 | 26,380 | 26,890 | 1.020 |
| G5-Ac² | 29,830 | 30,710 | 1.030 |
| G5-Ac²-FA | 32,380 | 35,470 | 1.095 |
| G5-Ac²-FA-OH | 34,460 | 40,580 | 1.178 |
| G5-Ac²-FA-OH-MTX^{e} | 36,730 | 36,960 | 1.006 |
| G5-Ac³ | 29,880 | 30,760 | 1.030 |
| G5-Ac³-FITC | 32,150 | 32,460 | 1.100 |
| G5-Ac³-FITC-OH | 34,380 | 34,790 | 1.012 |
| G5-Ac³-FITC-OH-MTX^{e} | 37,350 | 37,800 | 1.012 |
| G5-Ac³-FITC-FA | 34,710 | 35,050 | 1.010 |
| G5-Ac³-FITC-FA-OH | 36,820 | 37,390 | 1.016 |
| G5-Ac³-FITC-FA-OH-MTX^{e} | 39,550 | 39,870 | 1.008 |

### Example 4

### Dendrimer Characterization Via Gel Permeation Chromatography

The measured molecular weight of the G5 dendrimer of 26,380 g/mol is slightly lower than the theoretical one, (28,826 g/mol). These results indicate a deviation from the theoretical structure. The values in Table 1 were calculated utilizing GPC data for each conjugate (See, e.g., FIG. 8) and were calculated in order to derive the precise number of each functional group attached to the carrier. The average number of each functional molecule can be calculated by subtracting the M̅ₙ̅ value of the conjugate without the functional molecule in question from the M̅ₙ̅ value of the conjugate containing the functional molecule, and dividing by the molecular weight of the functional molecule. GPC eluograms of G5-Ac², G5-Ac²(82) —FA-OH-MTX^{e}, G5-Ac³(82) —FITC-OH-MTX^{e}, and G5-Ac2(82) —FITC-FA-OH-MTX^{e}, can be presented, with the R1 signal and laser light scattering signal overlapping at 900 (See, e.g., FIG. 8).

Based on GPC analysis, the number of conjugated FITC, FA, MTX, and glycidol molecules can be determined (See, e.g., FIG. 8: FITC: 5.8, FA: 5.7, MTX^{e}: 5-6, OH: 28-30). The number of conjugated molecules as determined by GPC was slightly higher than assumed; this is most probably due to the effect of citric acid in the eluent, which has varying effects dependent on the device in question. These values along with values obtained through analysis of NMR and UV spectra are utilized in combination to precisely determine the number of each conjugate molecules attached to the dendrimer.

Theoretical and defected chemical structures of the G5 PAMAM dendrimer are presented (See, e.g., FIG. 9). Side reactions such as bridging, as well as production of fewer arms per generation than theoretically expected, aid in producing a structure slightly different from the theoretical representation of the G5 PAMAM dendrimer. The defected chemical structure of a G5 PAMAM dendrimer exhibits missing arms from each generation, which can become problematic because they disturb the globular shape of the dendrimer, therefore affecting the number of functional molecules it is possible to attach and lessening the effects each functional molecule can have within the targeted cell(s).

### Example 5

### Characterization of Dendrimer Functional Groups

Acetylation of the dendrimer. Acetylation is the first requisite step in the synthesis of dendrimers. Partial acetylation is used to neutralize a fraction of the dendrimer surface from further reaction or intermolecular interaction within the biological system, therefore preventing non-specific interactions from occurring during synthesis and during drug delivery. Leaving a fraction of the surface amines non-acetylated allows for attachment of functional groups. Acetylation of the remaining amino groups results in increased water solubility (after FITC conjugation), allowing the dendrimer to disperse more freely within aqueous media with increased targeting specificity, giving it greater potential for use as a targeted delivery system as compared to many conventional mediums (Quintana et al., Pharm. Res. 19, 1310 (2002)).

Intensive dialysis, lyophilization and repeated membrane filtration using PBS and DI water were used to yield the purified, partially acetylated G5-Ac²(82) and G5-Ac³(82) PAMAM dendrimers (See, e.g., Majoros et al., Macromolec 36, 5526 (2003)). After conjugation of fluorescein isothiocyanate (FITC), and (FITC-FA) the dendrimer was fully acetylated again for an in vitro uptake study, following the same reaction sequence as found in (Wang, et al., Blood. 15, 3529 (2000)). Intensive dialysis, lyophilization and repeated membrane filtration were performed, yielding the fully acetylated G5-Ac¹(82)-FITC and G5-Ac¹(82)-FITC-FA PAMAM. As the degree of acetylation rises, the diameter of the dendrimer decreases, demonstrating an inverse relationship between the degree of acetylation and dendrimer diameter (See, e.g., Majoros et al., Macromolec 36, 5526 (2003)). The lower number of primary amines available for protonation (at a higher degree of acetylation, as compared to a lower degree) leads to a structure less impacted by charge-charge interactions, therefore leading to a more compact structure. The molecular weight however, has a parallel relationship to the degree of acetylation, as molecular weight increases as the degree of acetylation rises.

The PAMAM dendrimer was further characterized by H¹-NMR and HPLC (See, e.g., FIGS. 10 (A) and (B), respectively), by monitoring the eluted fractions by UV detection at 210 nm. H¹-NMR spectrum for G5-Ac displays the following: the peak appearing at 4.71 ppm is representative of D₂0, the peak at 3.67 ppm is representative of the external standard dioxane, and the peak at 1.89 ppm represents the methyl protons of the acetamide. Peaks 2.34 ppm, 2.55 ppm, 2.74 ppm, 3.04 ppm, 3.21 ppm, and 3.39 ppm are representative of the protons present in the acetylated dendrimer.

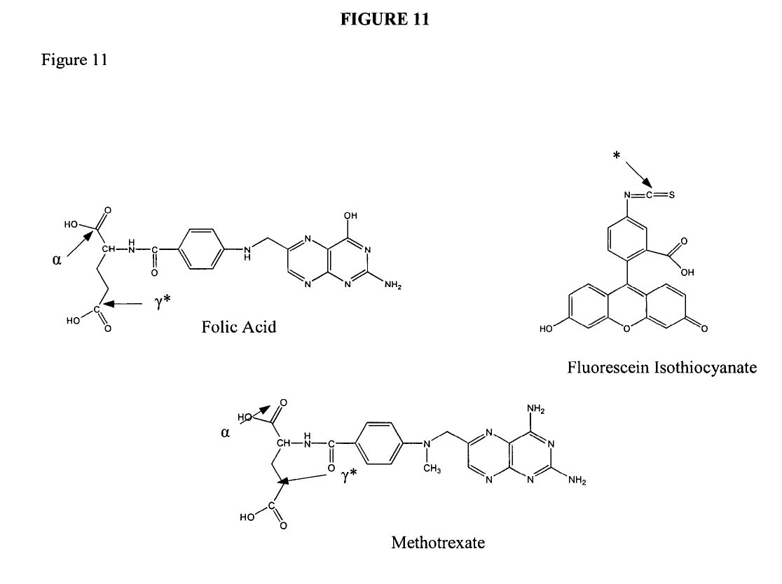

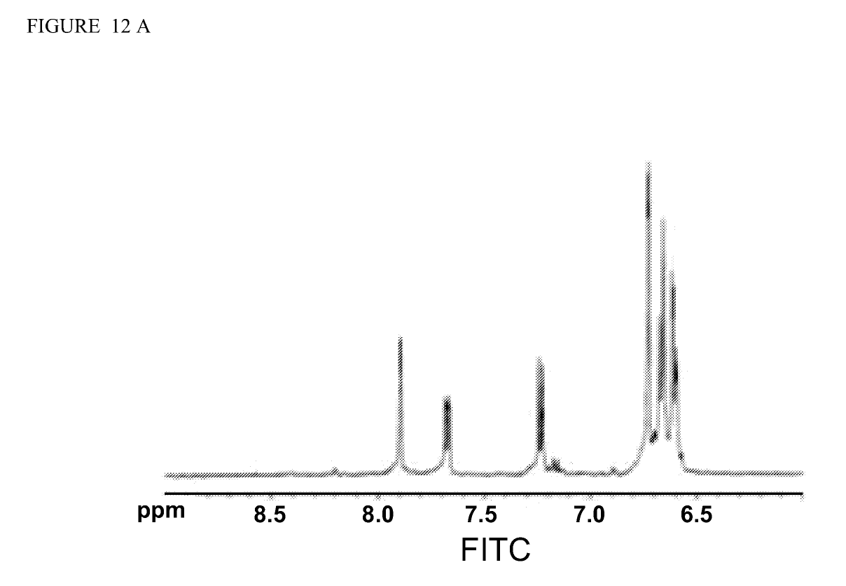

Structure of the functional groups. The structures of FITC, FA, and MTX are presented with the group to be attached to the dendrimer marked with an asterisk (See., e.g., FIG. 11, with the α- and γ-carboxyl groups labeled on both the FA and MTX molecules). When the γ-carboxylic group on FA is used for conjugation to the dendrimer, FA retains strong affinity towards its receptor, enabling FA to retain its ability to act as a targeting agent. Additionally, the γ-carboxylic group possesses higher reactivity during carboiimide mediated coupling to amino groups as compared to the α-carboxyl group (See, e.g., Quintana, et al., Pharm. Res. 19, 1310 (2002)).

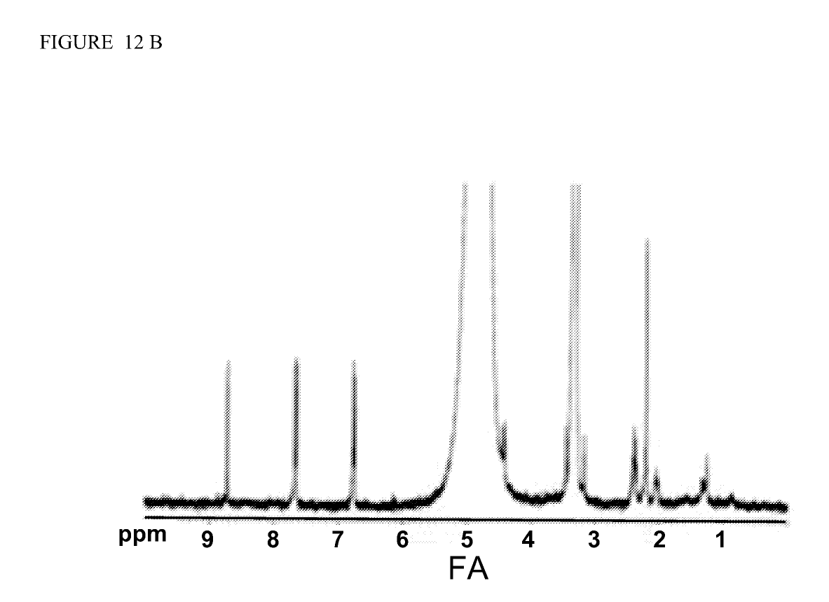

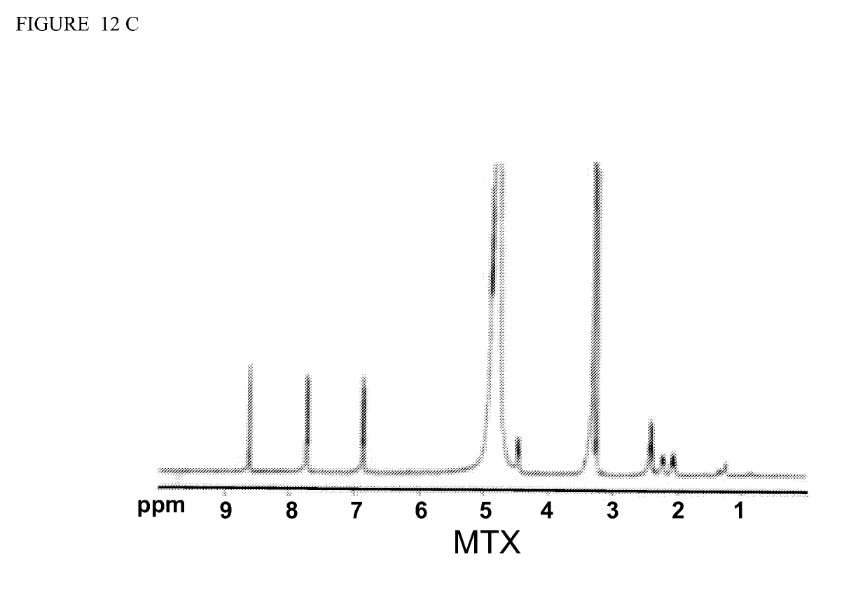

H¹-NMR of functional groups. In order to conclusively determine the numbers of each type of functional group attached to the dendrimer, the H-NMR of the functional groups themselves, and the H¹-NMR of the dendrimer conjugated to the functional groups must be compared. The H¹-NMR of the functional groups (See, for e.g., FIG. 12) the: FITC H¹-NMR-aromatic peaks: 7.9 ppm, 7.68 ppm, 7.23 ppm, 6.6 ppm, 6.65 ppm, 6.75 ppm; FA H¹-NMR-aromatic peaks: 8.73 ppm, 6.75 ppm, 7.65 ppm, D₂O at 4.85 ppm, CH₃OD at 3.3 ppm, alphatic peaks at: 2.15 ppm, 2.2 ppm, 2.4 ppm; and MTX H¹-NMR—aromatic peaks: 8.65 ppm, 8.75 ppm, 7.85 ppm, D₂0 at 4.8 ppm, CH₃OD at 3.35 ppm, aliphatic peaks at: 2.05 ppm, 2.25 ppm, 2.45 ppm.

### Example 6

### Conjugation of Functional Groups to Acetylated Dendrimer

Conjugation of fluorescein isothiocyanate to acetylated dendrimer. A partially acetylated G5-Ac³(82) PAMAM dendrimer was used for the conjugation of fluorescein isothiocyanate (FITC). The partially acetylated dendrimer was allowed to react with fluorescein isothiocyanate, and after intensive dialysis, lyophilization and repeated membrane filtration the G5-Ac³(82) —FITC product was yielded. The formed thiourea bond was stable during investigation of the devices.

Conjugation of folic acid to acetylated mono-functional dendrimer. Conjugation of folic acid to the partially acetylated mono-functional dendritic device was carried out via condensation between the γ-carboxyl group of folic acid and the primary amino groups of the dendrimer. This reaction mixture was added drop wise to a solution of DI water containing G5-Ac³(82) —FITC and was vigorously stirred for 2 days (under nitrogen atmosphere) to allow for the FA to fully conjugate to the G5-Ac³(82) —FITC. It is obvious that the a carboxyl group will participate in the condensation reaction, but its reactivity is much lower when compared to the γ carboxyl group. NMR was also used to confirm the number of FA molecules attached to the dendrimer. In the case that free FA is present within the sample, sharp peaks would appear in the spectrum. The ¹H NMR spectra of free FA (See, for e.g., FIG. 12) and G5-Ac³(82) —FITC-FA were taken. The broadening of the aromatic proton peaks in the G5-Ac³(82) —FITC-FA spectrum indicates the presence of a covalent bond between the FA and the dendrimer. Based on the integration values of the methyl protons in the acetamide groups and the aromatic protons in the FA, the number of attached FA molecules was calculated to be 4.5. The number of FA molecules (4.8), was determined by UV spectroscopy, utilizing the free FA concentration calibration curve.

Conjugation of MTX to acetylated two-functional dendrimer (via amide link). A control, MTX, tri-functional conjugate was synthesized from G5-Ac³(82) —FITC-FA. The similarity in structure of MTX, a commonly used anti-cancer drug, to FA allows for its attachment to G5-Ac³(82) —FITC-FA through the same condensation reaction used to attach FA to the primary amino groups. It was expected, from the molar ratio of the reactants, that five drug molecules would be attached per dendrimer. The ¹H NMR spectrum of the three-functional device was taken. The broadening of the aromatic proton peaks indicates the presence of a covalent bond between methotrexate and the dendrimer. Based on the integration values of the methyl protons in acetamide groups and the aromatic protons in the conjugated molecules, the number of attached methotrexate molecules was calculated to be five. MTX conjugation by an amide bond served as a control device for comparison of MTX conjugation through an ester bond. Attachment of methotrexate via an ester bond allows for relatively easier cleavage and release of the drug into the system as compared to linkage of MTX to the dendrimer by an amide bond.

Conjugation of glycidol to acetylated two-functional dendrimer. The conjugation of glycidol to the acetylated two-functional device was an important precursory step in order to attach MTX via an ester linkage and eliminate the remaining NH₂ to avoid any unwanted nonspecific targeting within the biological system. Conjugation of glycidol to the G5-Ac³(82) —FITC-FA converted all the remaining primary amino groups to alcohol groups, producing G5-Ac³(82) —FITC-FA-OH. For characterization purposes, conjugation of MTX to a glycidolated dendritic device containing FA or FITC produced G5-Ac²-FA-OH-MTX^{e 1}* and G5-Ac³-FITC-OH-MTX^{e 2}*(See, for e.g., FIGS. 13(A) and (B), the HPLC eluograms of each sample, respectively).

### Example 7

### Characterization of MTX Conjugated to Acetylated and Glycidylated Two-Functional Dendrimer Via Ester Link

The H¹-NMR for G5-Ac²-FA-OH-MTX^{e} is shown (See, for e.g., FIG. 14). The peaks representative of the aromatic protons of the conjugated device are indistinguishable from the aromatic peaks found in the H¹-NMR of free FA and MTX. Aromatic protons appear doubly 6.59 ppm, 7.53 ppm, and singly at 8.37 ppm. Comparison of the H¹-NMR of free FA and free MTX with that of the conjugated device shows that the aromatic regions overlap almost identically, therefore making it impossible to determine the location of the aromatic protons. The number of attached molecules of FA and MTX also affects the distributions of the peaks. The peak appearing at 4.70 ppm represents the solvent D₂O, the peak appearing at 3.67 ppm is representative of the external standard dioxane, and the peak appearing at 1.89 ppm is representative of the methyl protons of the acetamide groups. Peaks 2.31 ppm, 2.52 ppm, 2.71 ppm, and 3.26 ppm are representative of protons of the dendrimer.

### Example 8

### UV Spectra Characterization of Dendrimers

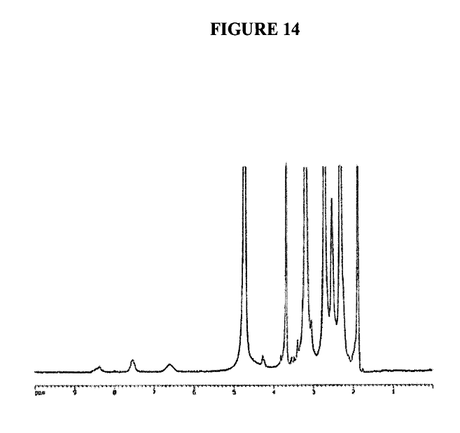

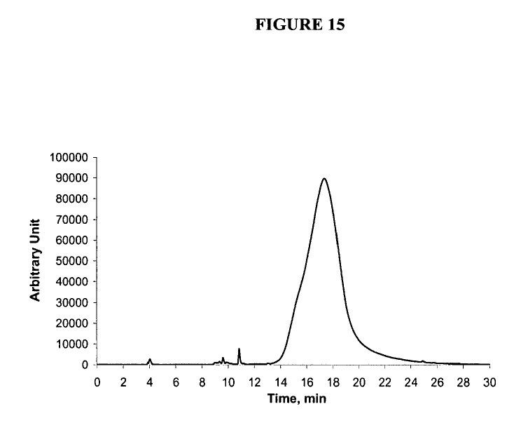

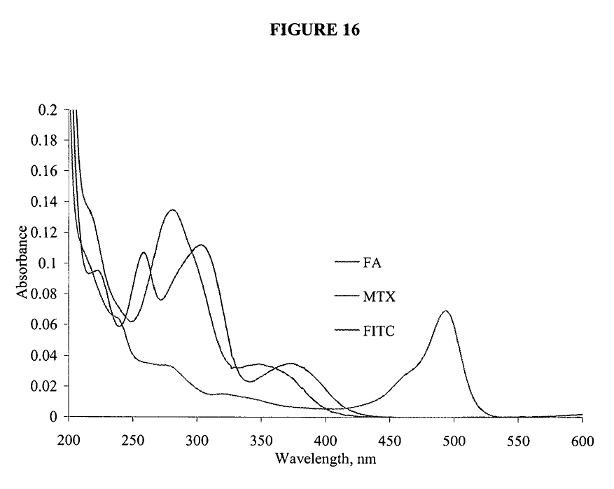

MTX conjugation via an ester linkage was tested for improved cleavage as compared to conjugation to the dendrimer via an amide linkage. The MTX is attached by use of EDC chemistry. The HPLC eluogram for G5-Ac-FITC-FA-OH-MTX^{e} at 305 nm is shown (See, for e.g., FIG. 15). The combined UV spectra for free FA, MTX and FITC can be compared to the for UV spectra of G5-Ac(82), mono-, bi- and tri-functional dendrimers (See, for e.g., FIGS. 16 and 17, respectively). UV spectra present defining peaks for FA at precisely 281 nm and 349 nm, for MTX on the order of 258 nm, 304 nm and 374 nm, and for FITC at 493 nm. The distinguishing peaks for FA, FITC and MTX visible (See, for e.g., FIG. 16) are dependent on the conjugation of each molecule to the dendrimer. Characterization of each device by comparison of UV spectra of free material and dendrimer-conjugated material was used to determine which function has been attached to the dendrimer.

### Example 9

### Cellular Uptake of Dendrimers

The fluorescence of the standard solutions of the conjugates G5-F1, G5-FITC-FA and G5-FITC-FA-MTX were measured using a spectrofluorimeter. A linear relationship between the dendrimer concentration and the fluorescence was observed at 10 to 1000 nM. The fluorescence of 100 nM solutions of G5-FITC, G5-FITC-FA and G5-FITC-FA-MTX were respectively 0.57, 0.23, and 0.11 spectrofluorimetric units. These differences in the fluorescence may be indicative of quenching due to the presence of FA and MTX on the dendrimer.

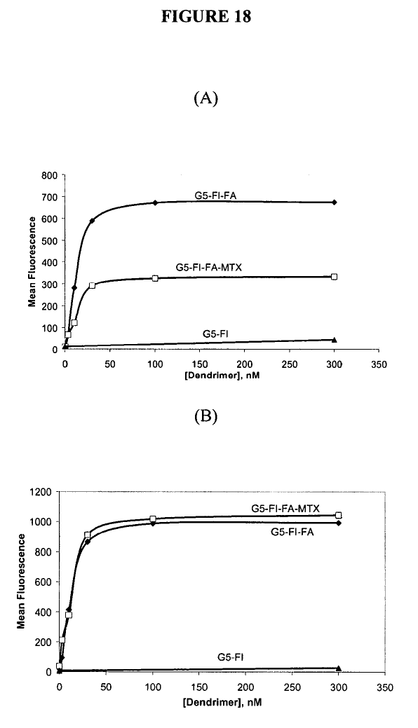

The cellular uptake of the dendrimers was measured in KB cells which express a high cell surface FA receptor (FAR). The FA-conjugated dendrimers bound to the cells in a dose-dependent fashion, with 50% binding at 10-15 nM for both the G5-FITC-FA and G5-FITC-FA-MTX, while the control dendrimer G5-FITC was not detected in the KB cells (See, e.g., FIG. 18A). Identical binding curves were obtained for the G5-FITC-FA and G5-FITC-FA-MTX when the fluorescence obtained was normalized for the quenching observed in the standard solutions of the dendrimers (See e.g., FIG. 18B). Analysis of the kinetics of the binding of the G5-FITC-FA-MTX (100 nM) showed that maximal binding was achieved within 30 minutes which is similar to reports for the binding of free folate.

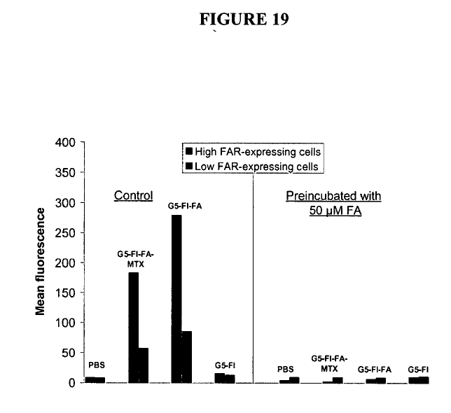

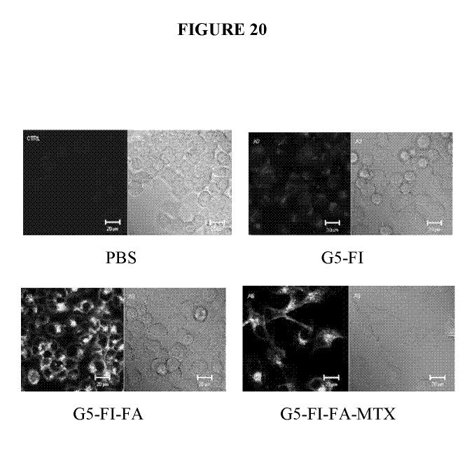

The effect of free FA on the uptake of the dendrimers was tested in KB cells that express both high and low FAR. The binding of the conjugates to the low FAR-expressing KB cells was 30% of that of the high FAR-expressing cells for both the G5-FITC-FA and G5-FITC-FA-MTX (See, for e.g., FIG. 19, left panel). 50 μM FA completely blocked the uptake of either targeted dendrimers (30 nM) in both the low- and high-FAR expressing cells (See, for e.g., FIG. 19, right panel). The binding and internalization of the dendrimers to KB cells was assessed by confocal microscopy. KB cells were incubated with 250 nM of the indicated dendrimers for 24 hours and confocal images were taken. Conjugates containing the targeting molecule FA internalized into KB cells within 24 h (See, e.g., FIG. 20). As compared to the cells treated with the control conjugates, the cells exposed to G5-FITC-FA-MTX were less adherent and rounded up, indicating cytotoxicity induced by the drug-conjugate.

### Example 10

### Functional Group Conjugated Dendrimers Inhibit Cell Growth

Because the binding of the conjugate to KB cells reaches maximal uptake within 1 h (Quintana et al, 2002. Pharmaceutical Research 19: 1310-1316), the effect of the G5-FI-FA-MTX on cell growth was initially tested by pre-incubation of cells with the conjugate for 1 h, followed by incubation in a drug-free medium for 5 d. Under such conditions, the conjugate failed to show any growth-inhibitory effect in KB cells. When the cells were pre-incubated with dendrimers for 4 h, there was a modest decrease of about 10% in cell growth as determined by XTT assay. The cytotoxicity measurements were therefore done by incubation with the dendrimer for a minimum of 24 h, a pre-incubation time period shown to induce significant cytotoxicity.

Time course and dose dependent inhibition of cell growth. Previous studies have shown that MTX-induced cytotoxicity is detectable in vitro only if the medium is completely deprived of FA (See, e.g., Sobrero & Bertino, Int. J. Cell Cloning 4, 51 (1986)). The effect of the trifunctional dendrimers on cell growth was tested in cells incubated in a FA-deficient medium. Cells were treated with 300 nM conjugates (equivalent of 1500 nM MTX) or 1500 nM free MTX for 1-4 days, and cell proliferation was determined by estimation of cellular protein content. Cells were treated for 2 days with different concentrations of the conjugates or free MTX (the conjugate concentration is given as MTX equivalents, with 5 MTX per dendrimer molecule). KB cells which express high and low FAR were incubated with 30 nM of the dendrimers for 1 hr at 37° C., rinsed, and the fluorescence of cells was determined by flow cytometric analysis (See. e.g., FIG. 21, left panel). Pre-incubation with 50 μM free FA for 30 min totally prevents cellular binding and uptake of the polymer conjugates (See. e.g., FIG. 21, left panel).

The inhibition of cell growth induced by the conjugates was also tested by XTT assay which is based on the conversion of XTT to formazan by the active mitochondria of live cells (See, e.g., Roehm et al, J Immunol Methods 142, 257 (1991)). The G5-FITC or G5-FITC-FA were not growth-inhibitory for the cells at 1, 2 or 3 days, whereas the G5-FITC-FA-MTX and free MTX showed time-dependent cytotoxicity (See e.g., FIG. 22). Hence, the G5-FITC-FA-MTX and free MTX inhibited cell growth in a time- and dose-dependent fashion, whereas the control dendrimers failed to inhibit the cell growth (See, for e.g., FIGS. 21 and 22).

### Example 11

### Folic Acid Rescues Cells from Methotrexate Induce Cytotoxicity

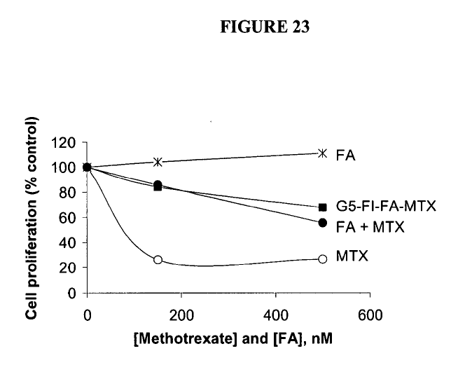

As growth inhibition induced by free MTX was higher than with the equimolar concentrations of MTX in the G5-FITC-FA-MTX below 1 μM (See, e.g., FIG. 21), it was tested whether the FA moiety in the G5-FITC-FA-MTX may be rescuing the cells from MTX-induced cytotoxicity. As the G5-FITC-FA-MTX preparation contained equimolar concentrations of MTX and FA, the effect of similar concentrations of free MTX and free FA on the inhibition of cell growth was determined. At equimolar concentrations of free FA and MTX, the FA reversed the inhibition of cell growth induced by MTX (See, e.g., FIG. 23). KB cells were treated with 150 or 500 nM MTX in the presence or absence of equimolar concentrations of free FA for 24 h. Cells were also treated with 30 and 100 nM G5-FI-FA-MTX (equivalent to 150 and 500 nM MTX) in parallel. The cells were rinsed to remove the drugs and incubated with fresh medium for an additional 6 d, and total cell protein was determined. The presence of 150 nM FA almost completely reversed the growth-arrest caused by 150 nM MTX. Moreover, the cytotoxicity induced by G5-FITC-FA-MTX (See, e.g., FIG. 23, filled square symbols) and equimolar combinations of FA and MTX (See, e.g., FIG. 23, filled circle symbols) was similar.

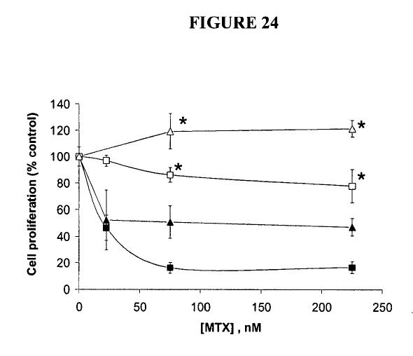

As free FA blocks the uptake of the dendrimers as well as rescues cells from MTX-induced cytotoxicity, the effect of pre-incubation of cells with excess FA on the anti-proliferative effect of G5-FITC-FA-MTX was tested. KB cells were exposed to different concentrations of the conjugate or free MTX for 24 h in the absence or presence of 50 μM FA. The incubation medium was removed, the cells were rinsed and incubated with fresh medium for 5 additional days in the absence of the drugs, and the XTT assay was performed (See, e.g., FIG. 24, □, ▪, represents cells treated with MTX; Δ, ▴, represents cells treated with G5-FITC-FA-MTX). Excess free FA not only blocked growth inhibition, but also increased cell growth 20% above that of the control cells (See, e.g., FIG. 24).

### Example 12

### Stability of Dendrimers

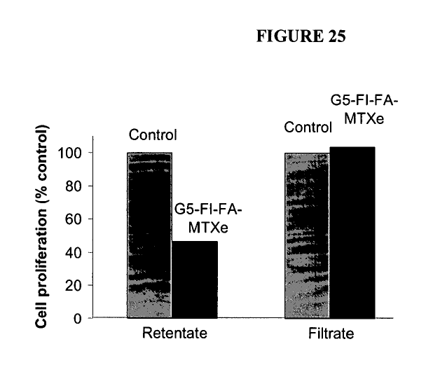

The stability of the dendrimer was tested in cell culture medium to check if MTX was released from the dendrimer prior to its entry into the cells. The G5-FITC-FA-MTX was incubated with cell culture medium for 1, 2, 4 and 24 h, and the incubation medium was filtered using a 10,000-MW cutoff ultrafiltration device. The effect of the retentate and the filtrate on the growth of the KB cells was tested. G5-FITC-FA-MTX was incubated with medium at 2 μM concentration for 24 h. The incubation medium was filtered through a Centricon 10K-MW cutoff filter. The retentate (adjusted to pre-filtration volume) and the filtrate were incubated with KB cells (at 200 nM conjugate, as determined from the concentration of the pre-filtration sample) for 2 days and the XTT assay was performed. Similar results were obtained for the retentate and filtrate obtained from the medium that had been pre-incubated with the dendrimers for 1, 2, and 4 hours. During the 24 h incubation time periods, the retentate was cytotoxic, whereas the filtrate failed to show any cytotoxicity (See, e.g., FIG. 25), indicating the lack of release of the free MTX from the conjugates. There was a slow release of the MTX after 24 h, reaching a maximum of 40-50% release in 1 week.

The anti-proliferative effect of the MTX-conjugates was compared to conjugates that lacked either the FA or the FITC molecule. KB cells were incubated with 30 nM of the conjugates (=150 nM effective MTX concentration) for 24 h and the incubation medium was removed. The cells were rinsed and incubated for an additional 5 d in fresh medium in the absence of the drugs, and the XTT assay was performed. The MTX-conjugated dendrimer that lacked FA failed to induce cytotoxicity, whereas the targeted dendrimer in the absence or presence of the dye molecule FITC induced cytotoxicity (See, e.g., FIG. 26).

### Example 13

### Use of Dendrimers to Target Tumors In Vivo

Compositions (e.g., dendrimers) and methods of the present invention were used to determine therapeutic response in an animal model of cancer (e.g., human epithelial cancer).

Materials and reagents. All reagents were obtained from commercial sources. Folic acid, penicillin/streptomycin, fetal bovine serum, collagenase type IV, TX100, bis-benzimide, FITC, methotrexate, hydrogen peroxide, acetic anhydride, ethylenediamine, methanol, dimethylformamide, and DMSO were purchased from Sigma-Aldrich (St. Louis, Mo.). Trypsin-EDTA, Dulbecco's PBS, and RPMI 1640 (with or without folic acid) were from Invitrogen (Gaithersburg, Md.). “Solvable” solution and hionic fluor were from Packard Bioscience (Downers Grove, Ill.). OCT embedding medium was from Electron Microscopy Sciences (Fort Washington, Pa.), 2-methyl butane from Fisher Scientific (Pittsburgh, Pa.), and 6-carboxytetramethylrhodamine (6-TAMRA) and Prolong were from Molecular Probes, Inc. (Eugene, Oreg.). Tritium-labeled acetic anhydride (CH₃CO)₂O (3H) (100 mCi, 3.7 GBq) was purchased from ICN Biomedicals (Irvine, Calif.). Methotrexate for injection was from Bedford Laboratories (Bedford, Ohio). Folic acid was solubilized in saline, adjusted to pH 7.0 with 1 N NaOH, and filter sterilized for injections.

Synthesis and characterization of PAMAM dendrimer conjugates. A G5 PAMAM dendrimer was synthesized and purified from low molar mass contaminants as well as higher molar mass dimers or oligomers (See, e.g., Majoros et al., Macromolecules 36, 5529 (2003)). The number average molar mass of the dendrimer was determined to be 26,530 g/mol by size exclusion chromatography using multiangle laser light scattering, UV, and refractive index detectors. The average number of surface primary amine groups in the dendrimer was determined to be 110 using potentiometric titration along with the molar mass. The polydispersity index, defined as the ratio of weight average molar mass and number average molar mass for an ideal monodisperse sample, equals 1.0. The polydispersity index of G5 dendrimer was calculated to be 1.032, indicating very narrow distribution around the mean value and confirming the high purity of the G5 dendrimer. The surface amines of G5 PAMAM dendrimers were acetylated with acetic anhydride to reduce nonspecific binding of the dendrimer. The ratio between the acetic anhydride and the dendrimer was selected to achieve different acetylation levels from 50 to 80 and 100 primary amines. After purification, the acetylated dendrimer was conjugated to an imaging agent (e.g., FITC or 6-TAMRA) for detection and imaging. The imaging-conjugated (e.g., dye-conjugated) dendrimer was then allowed to react with an activated ester of a targeting agent (e.g., folic acid), and the purified product of this reaction was analyzed by ¹H nuclear magnetic resonance (NMR) to determine the number of conjugated targeting agents (e.g., folic acid molecules). Subsequently, a therapeutic agent (e.g., methotrexate) was conjugated via an ester bond (See, e.g., Quintana et al, Pharm Res 19, 1310 (2002)).

Radiolabeled compounds were synthesized from G5-(Ac)₅₀-(FA)₆ or G5-(Ac)₅₀ using tritiated acetic anhydride (Ac-3H) (See, e.g., Malik et al., J Control Release 65, 133 (2000); Nigavekar et al., Pharm Res 21, 476 (2004); Wilbur et al., Bioconjug Chem 9, 813 (1998)). The tritiated conjugates, G5-³H-FA and G5-³H, were fully acetylated. The specific activity of the G5-NHCOC-3H and G5-FA-NHCOC-3H conjugates were 10.27 and 38.63 mCi/g, respectively. The residual free tritium was <0.3% of the total activity.

The quality of the PAMAM dendrimer conjugates was tested using PAGE, ¹H NMR, ³C NMR, and mass spectroscopy. Capillary electrophoresis was used to confirm the purity and homogeneity of the final products.

The folic acid-targeted conjugates specifically contain the following molecules: G5-(Ac)₈₂-(FITC)₅-(FA)₅, G5-(Ac)₈₂-(6-TAMRA)₃-(FA)₄, G5-(Ac)₈₂-(FITC)₅-(FA)-5-MTX₅, and G5-(Ac)₅₀-(Ac-3H)₅₄—(FA)₆, which were identified with the acronyms G5-FI-FA, G5-6T-FA, G5-FI-FA-MTX, and G5-3H-FA, respectively. The nontargeted controls contained the following molecules: G5-(Ac)₈₂-(FITC)₅, G5-(Ac)₈₂-(6-TAMRA)₃, G5-(Ac)₈₂-(FITC)-5-MTX₅, and G5-(Ac)₅₀-(Ac-3H)₅₄, which were identified with the acronyms G5-FI, G5-6T, G5-FI-MTX, and G5-3H, respectively.

Recipient animal and tumor model. Immunodeficient, 6- to 8-weekold athymic nude female mice (Sim:(NCr) nu/nu fisol) were purchased from Simonsen Laboratories, Inc. (Gilroy, Calif.). Five- to 6-week-old Fox Chase severe combined immunodeficient (SCID; CB-17/lcrCrl-scidBR) female mice were purchased from the Charles River Laboratories (Wilmington, Mass.) and housed in a specific pathogen-free animal facility at the University of Michigan Medical Center in accordance with the regulations of the University's Committee on the Use and Care of Animals as well as with federal guidelines, including the Principles of Laboratory Animal Care. Animals were fed ad libitum with Laboratory Autoclavable Rodent Diet 5010 (PMI Nutrition International, St. Louis, Mo.). Three weeks before tumor cell injection, the food was changed to a folate-deficient diet (TestDiet, Richmond, Ind.). For urine and feces collection, animals were housed in metabolic rodent cages (Nalgene, Rochester, N.Y.).

Tumor cell line. The KB human cell line, which overexpresses the folate receptor (See, e.g., Turek et al, J Cell Sci 106, 423 (1993)), was purchased from the American Type Tissue Collection (Manassas, Va.) and maintained in vitro at 37° C., 5% CO₂ in folate-deficient RPMI 1640 supplemented with penicillin (100 units/mL), streptomycin (100 μg/mL), and 10% heat-inactivated fetal bovine serum. Before injection in the mice, the cells were harvested with trypsin-EDTA solution, washed, and resuspended in PBS. The cell suspension (5×10⁶ cells in 0.2 mL) was injected s.c. into one flank of each mouse using a 30-gauge needle. In the biodistribution studies, the tumors were allowed to grow for 2 weeks until reaching ˜0.9 cm³ in volume. The formula chosen to compute tumor volume was for a standard volume of an ellipsoid, where V=4/3π (½ length×½ width×½ depth). With an assumption that width equals depth and k equals 3, the formula used was V=½×length×width². Targeted drug delivery using conjugate injections was started on the fourth day after implantation of the KB cells.

Biodistribution and excretion of tritiated dendrimer. Animals were injected via lateral tail vein with 0.5 mL PBS solution containing 174 μg G5-NHCOC-3H (1.8 ACi) or 200 μg G5-FA-NHCOC-3H (7.7 CCi). Both tritiumlabeled conjugates were delivered at equimolar concentrations of the modified dendrimer. At 5 minutes, 2 hours, 1 day, 4 days, and 7 days postinjection, the animals were euthanized and samples of tumor, heart, lung, liver, spleen, pancreas, kidney, and brain were taken. A third group of mice received a bolus of 80 μg free folic acid 5 minutes before injection with 200 μg G5-³H-FA. This 181 nmol concentration of free folic acid yields ˜150 μmol/L concentration in the blood compared with radiolabeled targeted dendrimer (G5-³H-FA), which yields ˜5 μmol/L concentration in the blood and is based on the 1.2 mL blood volume of a 20 g mouse. The mice were euthanized at 5 minutes, 1 day, and 4 days following injection, and tissues were harvested as above. Blood was collected at each time point via cardiac puncture. Each group included three to five mice. Urine and feces samples were collected at 2, 4, 8 and 12 hours and 1, 2, 3, and 4 days.

Radioactive tissue samples were prepared as described in Nigavekar et al, Pharrn Res 21, 476 (2004). The tritium content was measured in a liquid scintillation counter (LS 6500, Beckman Coulter, Fullerton, Calif.). The values of measured radioactivity were adjusted for the counting efficiency of the instrument and used to derive radioactivity (1 μCi=2.22×10⁶ dpm) per sample. These values were then normalized by tissue weight and the specific radioactivity of the conjugates was reported as a percentage of the injected dosage (% ID/g). The excreted radioactivity (dendrimer) via urine and feces was reported as a percentage of the injected dosage (% ID).

Biodistribution of fluorescent dendrimer conjugates. Mice were injected via lateral tail vein with 0.5 mL saline solution containing 0.2 mg G5-6T or G5-6T-FA conjugates. At 15 hours and up to 4 days postinjection, the animals were euthanized and samples of tumor were taken and immediately frozen for sectioning and imaging. Flow cytometry analysis was done with single-cell suspension isolated from tumor. Tumor was crushed, cell suspension filtered through 70 μm nylon mesh (Becton Dickinson, Franklin Lakes, N.J.), and washed with in PBS. Samples were analyzed using an EPICS XL flow cytometer (Coulter, Miami, Fla.). As determined by prior propidium iodine staining, only live cells were gated for analysis. Data were reported as the mean channel fluorescence of the cell population.

For confocal microscope imaging, tissue was dissected, embedded in OCT, and frozen in 2-methyl-butane in a dry ice bath. Sections (15 μm) were cut on a cryostat, thaw mounted onto slides, and stored at −80° C. until stained. After staining, the slides were fixed in 4% paraformaldehyde, rinsed in phosphate buffer (0.1 mol/L; pH 7.2), and mounted in Prolong. The images were acquired using a Zeiss 510 metalaser scanning confocal microscope equipped with a ×40 Plan-Apo 1.2 numerical aperture (water immersion) objective with a correction collar. The confocal image was recorded as 512×512×48 pixels with a scale of 0.45×0.45×0.37 μm per pixel. Each image cube was optically cut into 48 sections, and the sections that cut through the nucleus and cytoplasm were presented.

Delivery of targeted nanoparticle therapeutic. Twice weekly, SCID mice with s.c. KB xenografts, starting on day 4 after tumor implantation, received via the tail vein an injection of either targeted or nontargeted conjugate containing methotrexate, a conjugate without methotrexate, free methotrexate, or saline as a control. The compounds were delivered in a 0.2 mL volume of saline per 20 g of mouse. The single dose of methotrexate delivered each time equaled 0.33 mg/kg. The higher doses of 1.67 and 3.33 mg/kg free methotrexate were also tested. The conjugates were delivered at equimolar concentration of methotrexate calculated based on the number of methotrexate molecules present in a nanoparticle. The conjugate without methotrexate was delivered at equimolar concentration of dendrimer. In the initial trial, six groups of mice with five mice in each group received up to 15 injections. In the follow-up trial, mice received up to 28 injections dependent on their survival. The body weights of the mice were monitored throughout the experiment as an indication of adverse effects of the drug. Histopathology of multiple organs was done at the termination of each trial and each time mouse had to be euthanized due to toxic effects or tumor burden. Tissues from lung, heart, liver, pancreas, spleen, kidney, and tumor were analyzed. Additionally, cells were isolated from tumors, stained with targeted fluorescein-labeled conjugate, and tested for the presence of folic acid receptors using flow cytometer.

Statistical methods. Means, SD, and SE of the data were calculated. Differences between the experimental groups and the control groups were tested using Student's-Newman-Keuls' test and Ps<0.05 were considered significant.

Biodistribution of tritiated dendrimers. The biodistribution and elimination of tritiated G5-³H-FA was first examined to test its ability to target the folate receptor-positive human KB tumor xenografts established in immunodeficient nude mice. The mice were maintained on a folate-deficient diet for the duration of the experiment to minimize the circulating levels of folic acid (See, e.g., Mathias et al., J Nucl Med 29, 1579 (1998)). The free folic acid level achieved in the serum of the mice before the experiment approximated human serum levels (See, Belz et al., Anal Biochem 265, 157 (1998); Nelson et al., Anal Biochem 325, 41 (2004)). Mice were evaluated at various time points (5 minutes to 7 days) following i.v. administration of the conjugates. Two groups of mice received either control nontargeted tritiated G5-³H dendrimer or targeted tritiated G5-³H-FA conjugate (FIGS. 27A and B). The conjugates were cleared rapidly from the blood via the kidneys during the first day postinjection, with the G5-³H decreasing from 23.4% ID/g tissue at 5 minutes to 1.8% ID/g at 24 hours (FIG. 27A). The blood concentration of G5-³H-FA decreased from 29.1% ID/g at 5 minutes to 0.2% ID/g at 24 hours (FIG. 27B). In several organs, such as the lung, the tissue distribution showed a trend similar to blood concentrations with G5-³H decreasing from 9.7% ID/g at 5 minutes to 1.6% ID/g at 24 hours and G5-³H-FA decreasing from 9.6% ID/g at 5 minutes to 1.7% ID/g at 24 hours. Due to the high vascularity of the lung, conjugate levels measured at early time points likely reflect blood concentrations. Similar patterns of clearance were observed for the heart, pancreas, and spleen. These organs are known not to express folate receptor and do not show significant differences between the nontargeted and the targeted dendrimers. The concentrations of both G5-³H and G5-3H-FA in the brain were low at all time points, suggesting that the polymer conjugates did not cross the blood-brain barrier (FIGS. 27A and B). Although the kidney is the major clearance organ for these dendrimers, it is also known to express high levels of the folate receptor on its tubules. The level of nontargeted G5-³H in the kidney decreased rapidly and was maintained at a moderate level over the next several days (FIG. 27A). In contrast, the level of G5-³H-FA increased slightly over the first 24 hours most likely due to folate receptor present on the kidney tubules. This was followed by a decrease over the next several days as the compound was cleared through the kidney (FIG. 27B).

Both G5-³H and G5-³H-FA were rapidly excreted, primarily through the kidney, within 24 hours following injection. Incremental excretion of both compounds appeared entirely consistent with kidney retention of the conjugates (FIGS. 27A and B). Although both targeted and nontargeted conjugates also appeared in feces, it was in very low amounts. Whether any material was actually excreted in the feces was difficult to determine due to minor urine contamination of the feces. The cumulative clearance of the targeted G5-³H-FA over the first 4 days was lower than that of G5-³H, which may reflect retention of G5-³H-FA within tissues expressing folate receptors. The liver and KB tumor cells are known to express high levels of folate receptor. In these tissues, the concentrations of nontargeted G5-³H decreased rapidly with clearance of the dendrimer from the blood; the concentrations were maintained at a low level over the remaining days that the tissues were studied (FIG. 27A). In contrast, in both the liver and tumor, the targeted G5-³H-FA content increases over the first 4 days (FIG. 27B). This occurs during a time when blood levels of radioactive conjugate are low, suggesting specific uptake against a concentration gradient of dendrimer in these tissues, as opposed to the simple trapping of dendrimer through the vasculature.

The specificity of targeted drug delivery was further addressed in a group of mice receiving 181 nmol free folic acid before injection with G5-³H-FA (FIG. 27C). At 4 days after injection, significant attenuation in radioactivity related to the blocking of folate receptor with free folic acid was observed in tumor tissue that does not have the ability to excrete the dendrimer (FIG. 27C). This suggests that the difference in tumor concentrations between the targeted and the nontargeted polymer conjugates is due to the specific uptake of these molecules through the folate receptor overexpressed in the tumor. Distribution in all other tissues was not significantly altered by the delivery of free folic acid before the injection of the targeted conjugate.

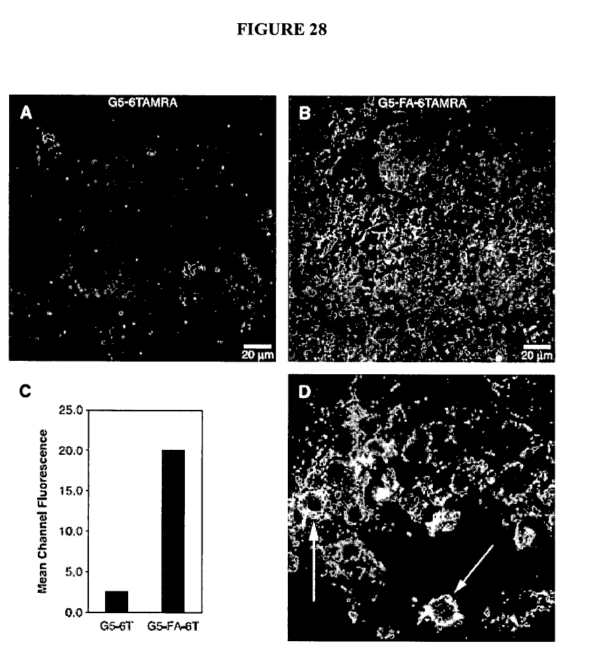

Targeting and internalization of fluorescent dendrimer conjugate. To further confirm and localize the dendrimer nanoparticles within tumor tissue, dendrimers conjugated with 6-TAMRA were employed. Confocal microscopy images were obtained of tumor samples at 15 hours following i.v. injection of the targeted G5-6T-FA and the nontargeted G5-6T conjugates (FIG. 28). The tumor tissue showed a significant number of fluorescent cells with targeted dye-conjugated dendrimer G5-6T-FA (FIG. 28B) compared with those with nontargeted dendrimer (FIG. 28A). Flow cytometry analysis of a single-cell suspension isolated from the same tumors showed higher mean channel fluorescence for tumor cells from mice receiving G5-6T-FA (FIG. 28C).

Confocal microscopy also showed that the conjugate is present in the tumors, attached to and internalized by many of the tumor cells (FIG. 28D). The optical overlapping sections were taken of the tissue slides from apical through medial to basal section. The medial section of tumor cells presented herein show fluorescence throughout the cytosol from the 6T of the conjugate, with the cell and nucleus boundary clearly visible (FIG. 28D).

Toxicity of dendrimer conjugates. All mice were observed for the duration of the studies for signs of dehydration, inability to eat or drink, weakness, or change in activity level. No gross toxicity, either acutely or chronically up to 99 days, was observed regardless of whether the dendrimer conjugate contained methotrexate. The weight was monitored throughout the experiment and no loss of weight was observed; in fact, the animals gained weight. At each time point, a gross examination and histopathology of the liver, spleen, kidney, lung, and heart were done. No morphologic abnormalities were observed on the histopathology examination. No in vivo toxicity was noted in any animal group following the dendrimer injection.

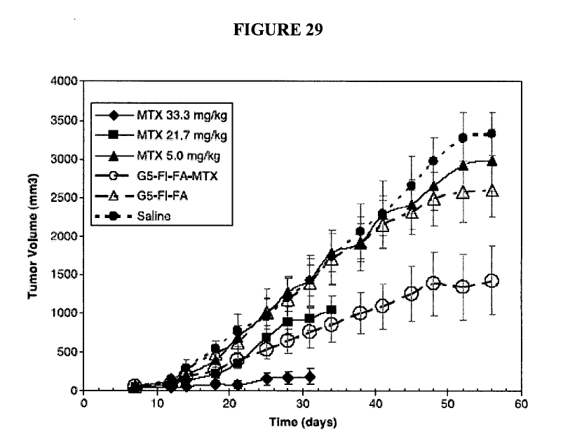

Targeted drug delivery to tumor cells through the folate receptor. The efficacy of different doses of conjugates was tested on SCID CB-17 mice bearing s.c. human KB xenografts and was compared with equivalent and higher doses of free methotrexate. Mice were maintained on the folic acid-deficient diet for 3 weeks before injection of the KB tumor cells to achieve circulating levels of folic acid that approach those in human serum and to prevent down-regulation of folate receptors on tumor xenografts (See, Mathias et al., J Nucl Med 29, 1579 (1998)). Six groups of SCID mice with five mice in each group were injected s.c. on one flank with 5×10⁶ KB cells in 0.2 mL PBS suspension. The highest total dose of G5-FI-FA-MTX therapeutic used equals 55.0 mg/kg and is equivalent to a 5.0 mg/kg total cumulative dose of free methotrexate (FIG. 29). The therapeutic dose of the conjugate was compared with three cumulative doses of free methotrexate equivalent to 33.3, 21.7, and 5.0 mg/kg accumulated in 10 to 15 injections based on mouse survival. Saline and the conjugate without methotrexate (G5-FI-FA) were used as controls.

The body weights of the mice were monitored throughout the experiment as an indication of adverse effects of the drug, and the changes of body weight showed acute and chronic toxicity in the highest and in the second highest cumulative doses of free methotrexate equal to 33.3 and 21.7 mg/kg, respectively. Although the two doses of free drug were affecting tumor growth, both became lethal by days 32 to 36 of the trial (FIG. 29). The remaining experimental groups had very uniform body weight fluctuations nonindicative of toxicity when compared with control groups with saline or conjugate without methotrexate. For the highest cumulative doses of free methotrexate used, histopathology analysis of the liver revealed advanced liver lesions, collections of inflammatory cells, and periportal inflammation. In contrast, neither the total accumulated dose of therapeutic conjugate equivalent to 5.0 mg/kg free methotrexate nor free methotrexate at the same dose were toxic (FIG. 29). Importantly, the therapeutic dose of conjugate that was equal to the lowest dose of free methotrexate used was as equally effective as the second highest dose of free methotrexate (21.7 mg/kg in 13 injections), whereas the free drug at this concentration had no effect on tumor growth (FIG. 29). The conjugate without methotrexate (G5-FI-FA) also had no therapeutic effect when compared with control injections of saline (FIG. 29). The liver slides from mice receiving the conjugate (G5-FI-FA-MTX) showed occasional periportal lymphocytes, indicating inflammation and single-cell necrosis that did not differ from that of control animals injected with saline.

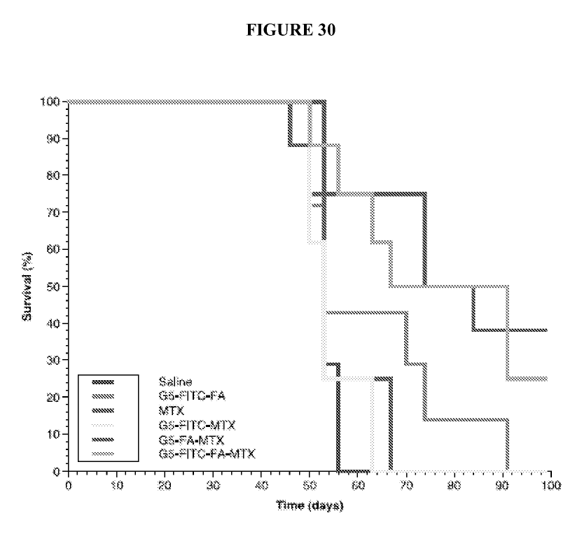

During a second 99-day trial, there was a statistically significant (P<0.05) slower growth of tumors that were treated with G5-FI-FA-MTX or G5-FA-MTX conjugate without FITC compared with those treated with nontargeted G5-FI-MTX conjugate, free methotrexate, or saline. The equivalent dose of methotrexate delivered with both targeted conjugates to the surviving mice was higher than the dose of free methotrexate because all of the mice receiving free methotrexate died by day 66 of the trial (FIG. 30). The survival of mice from groups receiving G5-FI-FA-MTX or G5-FA-MTX conjugate indicate that tumor growth based on the end-point volume of 4 cm3 can be delayed by at least 30 days (FIG. 30). This value indicates the antitumor effectiveness of the conjugate because it mimics clinical end-points and requires observation of the mice throughout the progression of the disease. Furthermore, a complete cure was obtained in one mouse treated with G5-FA-MTX conjugate at day 39 of the trial. The tumor in this mouse was not palpable for the next 20 days up to the 60th day of the trial. At the termination of the trial, there were three (of eight) survivors receiving G5-FA-MTX and two (of eight) survivors receiving G5-FI-FA-MTX. There were no mice surviving in the group receiving free methotrexate or in any other control group. Thus, in some embodiments, the present invention provides a composition comprising a dendrimer comprising a targeting agent, a therapeutic agent and an imaging agent. In preferred embodiments, the dendrimer is used for delivery, in a target specific manner, of a therapeutic agent (e.g., methotrexate) to tumor cells in vivo.

The effective dose of conjugate was not toxic based on weight change and the histopathology examination that was done. At the termination of both trials, histopathology examination did not reveal signs of toxicity in the heart and myopathy did not develop. Acute tubular necrosis in the kidneys was not observed in these animals. Analysis of tumor slides showed viable tumors with mild necrosis in the control and saline-injected animals, whereas the therapeutic conjugate caused severe to significant necrosis in tumors compared with an equivalent dose of free methotrexate. At the termination of the trial, tumor cells were evaluated for possible up-regulation of folic acid receptor in tumor compared with KB cells due to a long-term folic acid-depleted diet of mice. Flow cytometry analysis of tumor cells after staining with targeted fluorescein-labeled conjugate revealed that cells remained folic acid receptor positive but at two to five times lower level compared with original KB cell line.

### Example 14

### PAMAM-Dendrimer-RGD4C Peptide Conjugate Synthesis

Drug targeting is important for effective cancer chemotherapy. Targeted delivery enhances chemotherapeutic effect and spares normal tissues from the toxic side effects of these powerful drugs. Antiangiogenic therapy prevents neovascularization by inhibiting proliferation, migration and differentiation of endothelial cells (See, e.g., Los and Voest, Semin. Oncol., 2001, 28, 93). The identification of molecular markers that can differentiate newly formed capillaries from their mature counterparts paved the way for targeted delivery of cytotoxic agents to the tumor vasculature (See, e.g., Baillie et al., Br. J. Cancer, 1995, 72, 257; Ruoslahti, Nat. Rev. Cancer, 2002, 2, 83; Arap et al., Science, 1998, 279, 377). The αᵥβ₃ integrin is one of the most specific of these unique markers.

The αᵥβ₃ integrin is found on the luminal surface of the endothelial cells only during angiogenesis. This marker can be recognized by targeting agents that are restricted to the vascular space during angiogenesis (See, e.g., Brooks et al., Science, 1994, 264, 569; Cleaver and Melton, Nat. Med., 2003, 9, 661. High affinity αᵥβ₃ selective ligands, Arg-Gly-Asp (RGD) have been identified by phage display studies (Pasqualini et al., Nat. Biotech., 1997, 15, 542). The doubly cyclized peptide (RGD4C, containing two disulfide linkages via four cysteine residues) and a conformationally restrained RGD binds to αᵥβ₃ more avidly than peptides with a single disulfide bridge or linear peptides. There has been growing interest in the synthesis of poymer-RGD conjugates for gene delivery (See, e.g., Kunath et al., J. Gene. Med., 2003, 5, 588-599), tumor targeting (See, e.g., Mitra et al., J. Controlled Release, 2005, 102, 191) and imaging applications (See, e.g., Chen et al., J. Nuc. Med., 2004, 45, 1776).

In some embodiments, the present invention provides the synthesis of RGD4C conjugated to fluorescently labeled generation 5 dendrimer. Additionally the present invention provides the binding properties and cellular uptake of these conjugates.

Amine terminated dendrimers are reported to bind to the cells in a non-specific manner owing to positive charge on the surface. In order to improve targeting efficacy and reduce the non specific interactions, amine terminated G5 dendrimers were partially surface modified with acetic anhydride (75%×molar excess) in the presence of triethylamine as base (See e.g., Majoros et al., Macromolecules, 2003, 36, 5526. 4). The conjugate was purified by dialysis against PBS buffer initially and then against water. The use of 75 molar excess of acetic anhydride leaves some amine groups for further modification and prevents problems arising out of aggregation, intermolecular interaction and decreased solubility.

The degree of acetylation and purity of acetylated G5 dendrimer (G5-Ac) can be monitored using ¹H NMR spectroscopy. For detection of conjugates by flow cytometry or confocal microscopy a detectable probe (e.g., a fluorescent probe) can be used. For example, Alexa Fluor 488 (AF) can be used as a fluorescent label. The partially acetylated dendrimer was reacted with a 5 molar excess of Alexafluor-NHS ester as described in manufacturer's protocol to give fluorescently labeled conjugate (G5-Ac-AF). This conjugate was purified by gel filtration and subsequent dialysis. The number of dye molecules was estimated to be ˜3 per dendrimer by ¹H NMR and UV-vis spectroscopy as described in manufacturer's protocol (Molecular Probes).

The RGD peptide used in some embodiments of the present invention (RGD4C) has a conformationally restrained RGD sequence that binds specifically with high affinity to αᵥβ₃. The RGD binding site in the heterodimeric αᵥβ₃ integrin is located in a cleft between the two subunits. In order to keep the binding portion of the peptide exposed to the target site, an E-Aca (acylhexanoic acid) spacer was used to conjugate the peptide to the dendrimer. A protonated NH₂ terminus of the RGD-4C peptide is not essential for biological activity therefore. Thus, in some embodiments, the NH₂ terminus is capped with an acetyl group (See, e.g., de Groot et al., Mol. Cancer. Therap., 2002, 1, 901).

An active ester of the peptide was prepared by using EDC in a DMF/DMSO solvent mixture in presence of HOBt, and then this was added dropwise to the aqueous solution of the G5-Ac-AF. The reaction times are 2 h and 3 days, respectively. The amidation occurs predominantly on the acylhexanoic acid linker carboxylate group (e.g., A model reaction with 1.1 eq. allyl amine in DMSO gave the mono amidated product in 67% purity (HPLC). ESI-MS m/z 1282 (M+H)⁺). The partially acetylated PAMAM dendrimer conjugated with AlexaFluor and RGD peptide, G5-Ac-AF-RGD was purified by membrane filtration and dialysis. The ¹H NMR of the conjugate shows overlapping signals in the aromatic region for both the AlexaFluor and phenyl ring of peptide apart from the expected aliphatic signals for the dendrimer. The number of peptides was calculated to be 2-3 peptides per dendrimer based on MALDI-TOF mass spectroscopy.

MALDI-TOF MS has been widely used technique for characterization of surface functionalization of heterogeneously functionalized dendrimers (See, e.g., Woller et al., J. Am. Chem. Soc., 2003, 125, 8820-8826).

Mass spectra were recorded on a Waters TOfspec-2E, run in delayed extraction mode, using the high mass PAD detector and caliberated with BSA in sinapinic acid. To determine the functionalization of the dendrimer with peptide (m/z 29650 (M+H)⁺) of the starting material was subtracted from the (m/z 32770 (M+H)⁺) of the product.

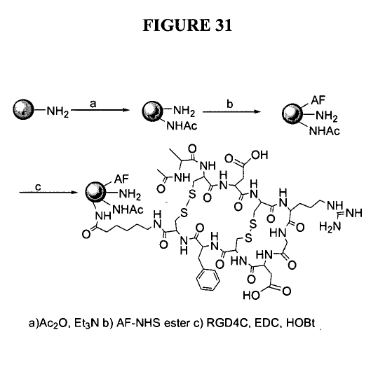

A schematic depicting the above described synthesis of G5-Ac-AF-RGD is shown in FIG. 31.

### Example 15

### In Vitro Targeting Efficacy of PAMAM-Dendrimer-RGD4C Peptide Conjugate

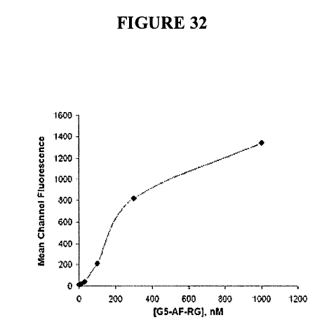

The cellular uptake of dendrimer-RGD4C conjugate was measured in Human umbilical vein endothelial cells (HUVEC) that express a high cell surface αᵥβ₃ receptor. In brief, HUVEC cells were cultured in RPMI medium supplemented with endothelial cell growth factor. The cells were treated with different concentrations of G5-Ac-AF-RGD conjugate and the uptake was monitored by flow cytometry. As shown in FIG. 32, flow cytometric analysis showed a dose-dependent and saturable binding to the HUVEC cells.

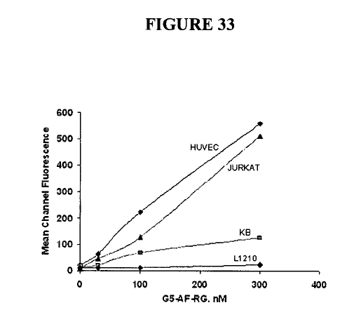

The binding of this conjugate to several different cell lines with varying levels of integrin receptor expression was also tested using flow cytometry (See, FIG. 33). The conjugate showed different binding affinities to various cell lines with HUVEC cells binding to the conjugate most effectively, followed by Jurkat cells. The human lymphocyte cell line Jurkat has previously been reported to have a large number of integrin receptors and was able to bind to RGD 4C peptide (See, e.g., Assa-Munt et al., Biochemistry, 2001, 40, 2373). The L1210 mouse lymphocyte line failed to bind the conjugate, whereas the KB cells showed only moderate binding.

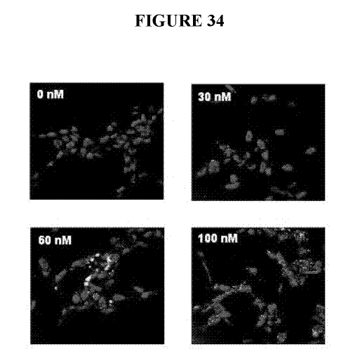

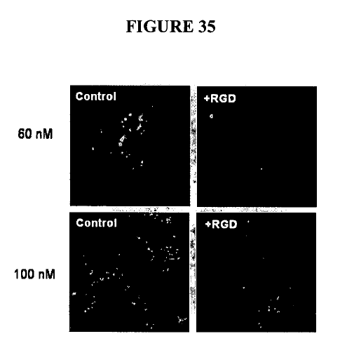

It is evident that the conjugate of the present invention shows variable specificities for cell lines having different levels of cell surface integrin receptor expression. The binding seen by flow cytometry was confirmed by confocal microscopic analysis. HUVEC cells treated with G5-AF-RGD4C (0, 30, 60, 100 nm) concentrations were washed and fixed with p-formaldehyde, the nuclei were counterstained with DAPI. It is evident from the appearance of fluorescence in confocal microscopic images in FIG. 34 that the uptake increases with the increasing concentration of the conjugate. The addition of free peptide inhibited the uptake of the conjugate by HUVEC cells to a significant level indicating receptor mediated uptake of the conjugate (See, FIG. 35).

In order to ascertain if polyvalent interaction shows stronger binding when compared to monovalent interaction, the binding affinity of G5-Ac-AF-RGD4C conjugate and RGD4C peptide were monitored on human integrin αvβ3 purified protein (Chemicon International, Inc. Temecula, Calif.) using a BIAcore instrument (BIAcore AB, Uppsala, Sweden). The obtained data for both analytes was analyzed by global fitting to a bivalent binding model using the BIAevaluation 3.2 software (BIAcore AB). The equilibrium dissociation constants (K_{D}) were calculated from the ratio of the dissociation and association rate constants (k_{off}/kₒₙ). The binding of the free RGD4C peptide to the human integrin αvβ₃ was very rapid in reaching a maximum binding of 10 RU. On the contrary, the binding of the G5-Ac-AF-RGD4C conjugate was less rapid, reaching a maximum binding of approximately 1500 RU. Both analytes showed different off-rates. The free RGD4C peptide rapidly dissociated from the ligand during the washing time with running buffer. The nanodevice dissociation was approximately 522 times slower as compared to the free peptide. Thus, the present invention provides a dendrimer wherein multiple peptide conjugation events on a single dendrimer exert a synergistic effect on binding efficacy.

Thus, the present invention provides PAMAM-dendrimer RGD4C peptide conjugates. In some embodiments, the dendrimer is taken up by cells expressing αᵥβ₃ receptors. Thus, in some preferred embodiments, the dendrimer conjugate is used to direct imaging agents and/or chemotherapeutics to angiogenic tumor vasculature.

### Example 16 cl Synthesis of G5-PMPA

Dendrimers comprising NAALADase inhibitors were synthesized. NAALADase (N-acetylated-alpha linked acidic dipeptidase) is a neuropeptidase that is highly homologous to prostate specific membrane antigen (PSMA). PSMA is a type II, integral membrane glycoprotein composed of a 19 amino acid intracellular domain containing the N-terminus, a 24 amino acid transmembrane region, and a 707 amino acid extracellular C-terminal domain (See, e.g., Murphy et al., J Urology 160, 2396 (1998)). Thus, the present invention provides dendrimers comprising an inhibitor of NAALADase for exponentially multiplying the binding affinity (e.g., polyvalency) of the dendrimers for use in targeting the dendrimers (e.g., comprising imaging or therapeutic agents) to cancer (e.g., prostate cancer) cells and tissue.

2-(Phosphonomethyl) pentanedioic acid (2-PMPA) is one such known inhibitor of NAALADase with a Ki value of 0.3 nM. Thus, a dendrimer conjugate was synthesized (G5-PMPA) with 2-PMPA as a targeting group for the specific targeting of cells or tissues expressing PSMA (e.g., prostate cancer cells).

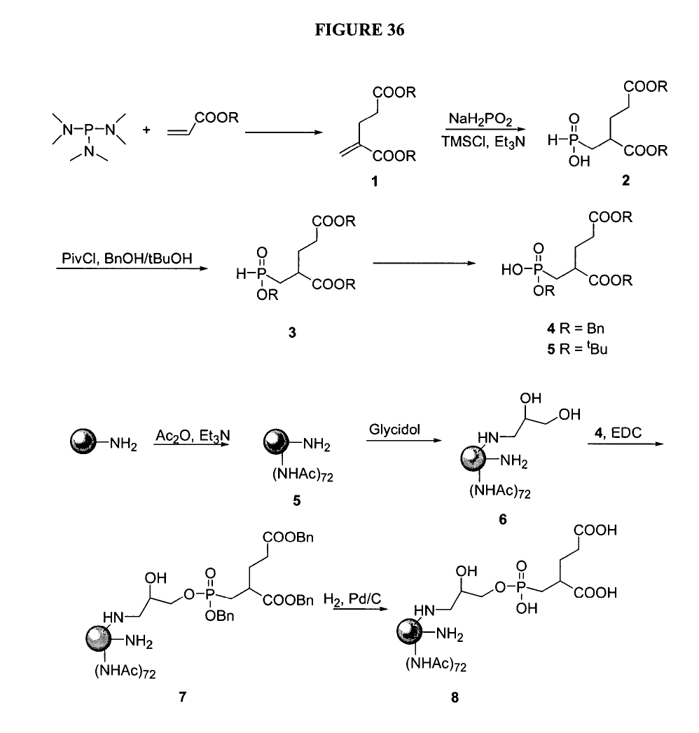

The G5 PAMAM dendrimer was synthesized and characterized at the Center for Biologic Nanotechnology, University of Michigan. Synthesis of ligands and dendrimer conjugates are described below and shown in FIG. 36. MeOH(HPLC grade), acetic anhydride, triethylamine, Benzyl acrylate, DMSO (99.9%), Hexamethyl phosphorous triamide, glycidol (racemic form, 96%), DMF, Sodium hypophosphite, Trimethylsilyl chloride, Pyridine, 1-(3-(Dimethylamino)-propyl)-3-ethylcarbodiimide HCl, Potassium hydrogen sulfate, Sodium thiosulfate, Palladium on carbon (10 wt %), Benzyl alcohol, and D₂O were all purchased from Aldrich and used as received.

Nuclear Magnetic Resonance Spectroscopy: ¹H and ¹³C NMR spectra were taken in D₂O and/or CD₃OD and were used to provide integration values for structural analysis by means of a Bruker AVANCE DRX 500 instrument.

UV Spectrophotometry. UV spectra were recorded using Perkin Elmer UV/VIS

Spectrometer Lambda 20 and Lambda 20 software, in PBS.

Surface Plasmon Resonance spectroscopy: The binding affinity of r-PSMA to G5-PMPA conjugate was studied using a BIAcore X instrument (BIAcore AB, Uppsala, Sweden). The obtained data for both analytes were analyzed by global fitting to a tetravalent binding model using the BIAevaluation 3.2 software (BIAcore AB).

Synthesis of Ligands. Ligands were synthesized according to the following process:

1. Dibenzyl 2-Methylenepentanedioate

2. Dibenzyl 2-Hydroxyphosphinoylmethyl-pentanedioate

3. 2-Benzyloxyphosphinoylmethyl-pentanedioic acid dibenzyl ester

4. 2-(Benzyloxy-hydroxy-phosphorylmethyl)-pentanedioic acid dibenzyl ester

5. Dibenzyl 2-Phosphonomethyl-pentanedioate

6. 2-Phosphonomethyl-pentanedioic acid

1. Dibenzyl 2-Methylenepentanedioate. Benzyl acrylate (50.0 g, 308.3 mmol) was heated to 100° C. while stirring under N₂. Hexamethyl phosphorous triamide (1.023 g, 6.3 mmol) was added dropwise while maintaining temperature of 135° C. for 30 mins. Reaction was stirred for 2.5 hours while cooling. The resulting orange liquid was loaded onto a silica column and eluted with 10:1 hexanes/ethyl acetate yielding 31.03 g of product.

2. Dibenzyl 2-Hydroxyphosphinoylmethyl-pentanedioate. To sodium hypophosphite hydrate (6.779 g, 77.06 mmol) in dichloromethane (100 ml) under N₂ was added Et₃N (18.73 g, 185.14 mmol) and TMSCl (22.09 g, 203.29 mmol) in 25 ml dichloromethane maintaining temperature below 101C. Reaction mixture was stirred for 30 min. 1 (5.00 g, 15.41 mmol) in dichloromethane (5 ml) was added keeping temp below 110° C. The mixture was allowed to warm to room temp and stirred for 18-20 h. Material was collected and reaction quenched by addition of 1N HCl. Organic layer was washed once with 1N HCl, and once with H₂O. Pooled aqueous layers were washed once with CH₂Cl₂ and organic phases pooled and dried over Na₂SO₄. Solvent was evaporated in vacuo. 5.61 g of crude product was obtained, and used without further purification.

3. 2-Benzyloxyphosphinoylmethyl-pentanedioic acid dibenzyl ester. 2 (2.52 g, 6.455 mmol) was dissolved in approx. 30 ml CH₂Cl₂. 3 ml pyridine was added to this solution to give (10:1 CH₂Cl₂/py). PivCl (0.856 g, 7.101 mmol) was added. Reaction mixture was stirred for 5 mins. BnOH (0.768 g, 7.101 mmol) was added. Reaction was stirred an additional 18 hours. Acid workup was performed with 1N HCl. Organic washes were pooled and solvent evaporated in vacuo. Compound was purified by silica gel chromatography and eluted with hexanes-ethyl:acetate (1:5) yielding 1.43 g of product.

4. 2-(Benzyloxy-hydroxy-phosphorylmethyl)-pentanedioic acid dibenzyl ester. 3 (1.420 g, 2.955 mmol) was dissolved in acetonitrile-water (20 mL, 1:1 by volume). Sodium periodate (0.790 g, 3.694 mmol) was added at room temperature and mixture heated to 50° C. and stirred for 3 hrs. Reaction mixture was then concentrated in vacuo. The residual mixture was dissolved in EtOAc (150 mL) and washed with saturated aq. KHSO₄ (100 mL), 0.2 M NaS₂O₃ (100 mL) and twice with brine respectively. Organic layer was dried over Na₂SO₄ and solvent removed in vacuo to yield 1.178 g of product.

5. Dibenzyl 2-Phosphonomethyl-pentanedioate. 2 (1.000 g, 2.562 mmol) was dissolved in acetonitrile-water (12 mL, 1:1 by volume). Sodium periodate (0.685 g, 3.202 mmol) was added at room temperature and mixture heated to 50° C. and stirred for 3 hrs. Reaction mixture was then concentrated in vacuo. The residual mixture was dissolved in EtOAc (75 mL) and washed with saturated aq. KHSO₄ (75 mL), 0.2 M NaS₂O₃ (75 mL) and twice with brine respectively. Organic layer was concentrated, then dried over Na₂SO₄ and solvent removed in vacuo to yield 0.847 g of product.

6. 2-Phosphonomethyl-pentanedioic acid. 5 (0.4307 g, 1.0598 mmol) was dissolved in methanol (2 ml) and added to 20 ml H₂O. 10% Pd/C (0.0861 g) was added to reaction vessel. Vessel was placed on Parr hydrogenation apparatus. Reaction was carried out under H₂ (Q50 psi) for 20 hours while shaking. Pressure was relieved and Pd/C filtered off. Pd/C was flushed with water. Resulting mixture was lyophilized yielding 0.2085 g of dried product.

Synthesis of dendrimer conjugates. Dendrimer conjugates were synthesized according to the following process:

7. G5 carrier

8. G5-Ac(72)

9. G5-Ac(72)-AF488

10. G5-Ac(72)-AF488-OH

11. G5-Ac(72)-AF488-OH-bPMPA

12. G5-Ac(72)-AF488-OH-PMPA

7. G5 carrier. The PAMAM G5 dendrimer was synthesized and characterized at the Center for Biologic Nanotechnology, University of Michigan. PAMAM dendrimers are composed of an ethylenediamine (EDA) initiator core with four radiating dendron arms, and are synthesized using repetitive reaction sequences comprised of exhaustive Michael addition of methyl acrylate (MA) and condensation (amidation) of the resulting ester with large excesses of EDA to produce each successive generation. Each successive reaction therefore theoretically doubles the number of surface amino groups, which can be activated for functionalization. The synthesized dendrimer has been analyzed and the molecular weight has been found to be 26,380 g/mol by GPC and the average number of primary amino groups has been determined by potentiometric titration to be 110.

8. G5-Ac(72). 7 (0.140 g, 0.0053 mmol) was dissolved in anhydrous MeOH (15 ml). Et₃N (0.0427 g, 0.4222 mmol) was added while stirring. After 30 mins. acetic anhydride (0.0431 g, 0.4222 mmol), diluted in anhydrous MeOH (15 ml), was added dropwise while vigorously stirring. Reaction was stirred overnight at room temp. Solvent was evaporated in vacuo and resulting material dissolved in H₂O. Compound was dialyzed in 10,000 MWCO membrane 4 times against PBS followed by 4 times against H₂O. Material was lyophilized. The average number of acetyl groups (72) has been determined based on ¹H NMR calibration.

9. G5-Ac(72)-AF488. 8 (34.56 mg, 1.17 μmol) was dissolved in 3.0 ml PBS. Alexa fluor 488-NHS ester (3.0 mg, 4.67 μmol) was dissolved in 600 μl DMSO and added dropwise to reaction mixture while stirring under N₂. Reaction was stirred overnight. Reaction mixture was concentrated by membrane filtration and conjugate was separated from free dye on G-25 Sephadex column. Pooled fractions were concentrated and buffer exchanged by membrane filtration. Conjugate was lyophilized.

10. G5-Ac(72)-AF488-OH. 9 (27.86 mg, 0.920 μmol) was dissolved in 3 ml H₂O in a stirred reaction vial under N₂. Seventy-fold excess of glycidol (4.75 mg, 0.0642 mmol) was added to reaction vial. Reaction was stirred for 4 hours. Conjugate was purified by membrane filtration (2× with PBS and 4× with H₂O) through 10,000 MWCO centricon device. Conjugate was then lyophilized.

11. G5-Ac(72)-AF488-OH-bPMPA. 4 (3.055 mg, 6.154 μmol) was dissolved in anhydrous DMSO (550 μl). Phosphonic acid was activated by stirred reaction with 2.5 eq. EDC (2.949 mg, 15.385 μmol) and DMAP (1.880 mg, 15.385 μmol) under N₂. After mixing 1 hr, activated phosphonic acid was added dropwise to 10 (13.36 mg, 0.4103 μmol) in H₂O (2.5 ml) while stirring. Conjugate was purified by membrane filtration (1×25/75 DMSO/PBS; 2×PBS; 4×H₂O) through 10,000 MWCO centricon device.

12. G5-Ac(72)-AF488-OH-PMPA. 11 (8.91 mg) was dissolved in water (˜20 ml). 10% Pd/C (80 mg) was added to reaction vessel. Vessel was placed on Parr hydrogenation apparatus. Reaction was carried out under H₂ (˜50 psi) for 24 hours while shaking. Pressure was relieved and Pd/C filtered off. Pd/C was flushed with H₂O. Material was lyophilized.

### Example 17

### Affinity of G5-PMPA for Prostate Specific Membrane Antigent (PSMA)

Affinity data for G5-PMPA binding to PSMA was obtained using Biacore. rPSMA (8765 RU) was coupled to a Biacore CM5 sensor chip using an amine coupling procedure. Theoretical Rₘₐₓ was calculated for tetravalent binding of G5-PMPA (MW 33 kDa) to immobilized rPSMA (MW 93 kDa); Rₘₐₓ=12,440 RU. 200 nM G5-PMPA bound strongly to rPSMA, with a calculated K_{D} in the sub-nanomolar range.

### Example 18

### Specific Targeting of Functionalized Iron Oxide Nanoparticles into Tumor Cells Through Folate Receptor-Mediated Endocytosis

A) Synthesis, Fabrication and Characterization of Functionalized Iron Oxide Nanoparticles (NPs)

Iron oxide (Fe₃O₄) NPs were synthesized by controlled co-precipitation of Fe(II) and Fe(III) ions (See, e.g., Mikhaylova, M. et al. Chem. Mater. 16, 2344-2354 (2004)). Briefly, 25 mL of 1 M FeCl₃.6H₂O, 0.5 M FeCl₂.4H₂O and 0.4 M HCl mixture solution was prepared in water under vigorous stirring. The co-precipitation of Fe₃O₄ NPs was carried out in a three-neck round-bottom flask. The above mixture solution was added to 250 mL of 0.5 M NaOH, which was preheated to 80° C. before the co-precipitation reaction. The reaction was protected under N₂ atmosphere and was vigorously stirred. Black powder was collected by sedimentation with the help of an external magnetic field and washed several times with water until stable ferrofluid was obtained. Finally, the particles were redispersed in water.

Amine-terminated G5 dendrimer (G5.NH₂) was conjugated with fluorescein isothiocyanate (FI) or both FI and folic acid (FA) moieties, according to previously published reports (See, e.g., Majoros et al., J. Med. Chem. 48, 5892-5899 (2005); Shi, X. et al. Analyst 131, 374-381 (2006); Quintana, A. et al. Pharm. Res. 19, 1310-1316 (2002) and U.S. Pat. App. Nos. 60/604,321, 60/690,652, and 60/707,991, each of which is hereby incorporated by reference in their entireties). Briefly, G5.NH₂ (60 mg, 0.00225301 mmol) was dissolved in anhydrous DMSO (24 mL). To the above solution was added dropwise a solution of FI (4.4 mg, 0.00563275 mmol) in DMSO (24 mL) under vigorous stirring at room temperature. The reaction was stopped after 24 h. The mixture was dialyzed against PBS buffer and water through a 10,000 MWCO membrane. Lyophilization gave G5.NH₂-FI as an orange solid (60.6 mg, yield 94.0%). For the synthesis of G5.NH₂-FI-FA, FA (3.7 mg, 0.0084004 mmol) and EDC (9.3 mg, 0.021001 mmol) were dissolved in DMSO (3 mL) and the mixture was stirred at room temperature for 3 h. The resulting solution was added dropwise to a solution of G5.NH₂-FI (30 mg, 0.0010501 mmol) in DMSO (12 mL) under vigorous stirring at room temperature. After 3 days, the reaction mixture was dialyzed through a 10,000 MWCO membrane against PBS buffer and water, followed by lyophilization to give G5.NH₂-FI-FA (31.2 mg, yield 96.4%). The G5.NH₂-FI and G5.NH₂-FI-FA conjugates were characterized by ¹H NMR and matrix-assisted laser desorption ionization-time of flight (MALDI-TOF) mass spectrometry.

The numbers of FI and FA moieties conjugated onto each G5 dendrimer can be estimated by comparing the differences between the integration values of ¹H NMR signals associated with dendrimers and the FI and FA moieties. The average numbers of FI and FA moieties conjugated onto each G5 dendrimer were estimated to be 4.5 and 4.8, respectively. The molecular weights of G5.NH₂-FI and G5.NH₂-FI-FA conjugates were determined to be 29,900 and 33,600, respectively.

Next, LbL assembly of oppositely charged poly(styrene sulfonate) (PSS) and dendrimers was performed (See, e.g., Schneider, G. & Decher, G. Nano Lett. 4, 1833-1839 (2004); Gittins, D. I. & Caruso, F. J. Phys. Chem. B 105, 6846-6852 (2001); Khopade, A. J. & Caruso, F. Nano Lett. 2, 415-418 (2002); Khopade, A. J. & Caruso, F. Biomacromolecules 3, 1154-1162 (2002)). Briefly, a solution of Fe₃O₄ NPs (5 mg in 0.5 mL water) was added with 1 mL of a PSS solution (2 mg/mL, containing 0.5 M NaCl) with occasional shaking. After adsorption of PSS for 20 min, the suspension was centrifuged at 8,000 rpm for 10 min. The supernatant was then carefully removed, and the coated Fe₃O₄ NPs were washed by three alternate cycles of centrifuging and resuspending the particles in pure water. Then 1 mL of G5.NH₂-FI-FA solution (1 mg/mL, containing 0.5 M NaCl) was added into the PSS-modified Fe₃O₄ NP suspension and purified in the same manner. The G5.NH₂-FI-FA/PSS-coated Fe₃O₄ NPs were subjected to an acetylation reaction to neutralize the remaining amine groups of G5.NH₂-FI-FA dendrimers (See, e.g., Majoros et al., Macromolecules 36, 5526-5529 (2003)). The Fe₃O₄/PSS/G5.NH₂-FI-FA NPs (in 1 mL water) were added with triethylamine (2.48 μL) and mixed well. Then, a methanol solution (0.1 mL) containing 1.82 mg acetic anhydride was added dropwise into the Fe₃O₄/PSS/G5.NH₂-FI-FA NP/triethylamine solution. The reaction mixture was vigorously shaken for 24 h. The formed Fe₃O₄/PSS/G5.NHAc-FI-FA NPs were purified by 4 cycles of centrifugation/redispersion in water. For biological testing, the NPs were transferred to PBS buffer solution by centrifugation and redispersion. The concentration of the stock solution of Fe₃O₄/PSS/G5.NHAc-FI-FA NPs was kept at 20 mg/mL Fe₃O₄ in PBS. The NPs were stored at 4° C. before biological testing. The control Fe₃O₄/PSS/G5.NHAc-FI NPs without FA conjugation were prepared in the same manner as the procedure used to prepare Fe₃O₄/PSS/G5.NHAc-FI-FA NPs.

The surface potential of functionalized Fe₃O₄ NPs was measured by a Malvern Zetasizer Nano ZS model ZEN3600 (Worcestershire, UK) equipped with a standard 633 nm laser. The size and morphology of Fe₃O₄ NPs were characterized by a Philips CM-100 TEM equipped with a Hamamatsu Digital Camera ORCA-HR operated using AMT software (Advanced Microscopy Techniques Corp, Danver, Mass.). The operation voltage was kept at 60 kV. TEM samples were prepared by deposition of a diluted particle suspension (5 μL) onto a carbon-coated copper grid and air-dried before the measurement. Stained specimens were prepared by depositing the sample solutions on the grid and inverting the grid on a drop of aqueous phosphotungstic acid solution that had been neutralized with NaOH (2% mass fraction of phosphotungstic acid). The grid was then blotted on filter paper and air-dried.

KB cell culture. KB cells (ATCC, CLL17, Rockville, Md.) were continuously grown in two 24-well plates, one in FA-free media and the other in regular RPMI 1640 medium (Gibco/BRL, Gaithersburg, Md.) supplemented with penicillin (100 units/mL) (Sigma, St. Louis, Mo.), streptomycin (100 μg/mL) (Sigma, St. Louis, Mo.), 10% heat-inactivated FBS, and 2.5 μM FA. The cells grown in FA-free media express high-level FAR, while the cells grown in FA-containing media express low-level FAR.

Flow cytometry. Approximately 1×10⁵ cells per well were seeded in 24-well plates the day before the experiments. An hour before initiating an experiment, the cells were rinsed four times with serum-free and FA-deficient RPMI 1640 media. Fe₃O₄/PSS/G5.NHAc-FI and Fe₃O₄/PSS/G5.NHAc-FI-FA NPs were added at Fe concentrations of 0-180 μg/mL. After 1 h incubation, KB cells with both high- and low-level FAR were trypsinized and suspended in PBS containing 0.1% bovine serum albumin, and then analyzed using a Coulter EPICS-XL MCL Beckman-Coulter flow cytometer. The FL1-fluorescence of 10,000 cells was measured, and the mean fluorescence of gated viable cells was quantified using Expo32 software (Beckman-Coulter, Miami, Fla.).

Confocal laser scanning microscopy. Confocal microscopic analysis was performed in cells plated on a plastic cover-slip using an Olympus FluoView 500 laser scanning confocal microscope (Melville, N.Y.). FI fluorescence was excited with a 488 mm argon blue laser and emission was measured through a 505-525 barrier filter. The optical section thickness was set at 5 μm. The KB-HFAR cells were incubated with Fe₃O₄/PSS/G5.NHAc-FI or Fe₃O₄/PSS/G5.NHAc-FI-FA NPs for 2 h. Then the cells were washed with PBS. The nuclei were counterstained with 1 μg/mL of Hoescht33342, using a standard procedure. Samples were scanned on an Olympus IX-71 inverted microscope, using a 60× water immersion objective and magnified with FluoView software.

Transmission electron microscopy (TEM). The uptake of functionalized Fe₃O₄ NPs was further examined by a Phillips CM 100 TEM microscope operating at a voltage of 60 kV. Images were recorded using a Hamamatsu digital camera controlled by AMT (Advance Microscopy Technology) software. The KB-HFAR cells were incubated with Fe₃O₄/PSS/G5.N-HAc-FI or Fe₃O₄/PSS/G5.N-HAc-FI-FA NPs for 2 h. The medium was then removed and the cells were washed with Sorenson buffer and fixed at room temperature for 1 h using 2.5% of glutaraldehyde in Sorenson buffer. The cells were rinsed 3 times with Sorenson buffer, resuspended in the same medium, and post-fixed using 1.0% osmium tetroxide for 1 h. After additional washing in buffer, the cells were dehydrated in a series of ethanol solutions of 30%, 50%, 70%, 95%, and 100%. Samples were further infiltrated using the following sequence of mixtures of 100% ethanol and Epon: 3 parts of ethanol+1 part resin (for 1 h), 1 part of ethanol+1 part resin (for 1 h), 1 part of ethanol+3 parts resin (overnight), full-strength resin (4 h), and full-strength resin (overnight). After the third change of resin, polymerization was performed and sections with a thickness of 75 nm were obtained using a Reichart Ultramicrotom. Sections were mounted on 200 mesh copper grids before TEM measurements.

In vitro magnetic reasonance (MR) relaxometry and imaging. 5×10⁶ KB-HFAR cells were incubated with Fe₃O₄/PSS/G5.NHAc-FI and Fe₃O₄/PSS/G5.NHAc-FI-FANPs with Fe concentrations of 22.5, 45, and 90 g/mL for 30 min at 0° C. The cells were then washed with PBS buffer three times. The cells were centrifuged to prepare pellets for MR imaging. Studies were performed with a 2.0 T Varian Unity/Inova system (Palo Alto, Calif.) using home-built RF coils. One hundred microliters of PBS was added to each cell pellet, and the cells were suspended by gentle shaking. T1 and T2 of the cell suspensions were measured in each sample vial with inversion recovery and CPMG pulse sequences, respectively. The cells were then allowed to settle and a phantom was constructed consisting of all of the sample vials. A spin-echo image (TRITE 2000/8 ms) with a 2.0 mm-slice thickness and an in-plane resolution of 0.312 mm was acquired through the plane of the cells.

Cell toxicity analysis. An MTT (3-(4,5-dimethylthiazol-2-yl)-2,5-diphenyltetrazolium bromide) assay was used to quantify the viability of cells. Briefly, 1×10⁴ KB cells per well were seeded into a 96-well plate and incubated with 45 μg/mL of Fe₃O₄/PSS/G5.NHAc-FI or Fe₃O₄/PSS/G5.NHAc-FI-FA for 24 h before the addition of MTT. The percentage of living cells was defined as absorbance of the treated well/absorbance of the control well×100% with the OD 570 nm measured by automated plate reader. The morphology of cells treated with functionalized Fe₃O₄ NPs with Fe concentrations 0, 45, 225, and 360 μg/mL were observed by a Leica DMIRB fluorescent inverted microscope. The magnification was set at 200× for all samples.

B) Self-Assembled Bilayers of PSS/G5.NHAc-FI and PSS/G5.NHAc-FI-FA on Fe₃O₄

Iron oxide (Fe₃O₄) NPs were synthesized by controlled co-precipitation of Fe (II) and Fe (III) ions as described above. The synthesized Fe₃O₄ NPs (8.4±1.4 nm in diameter as verified by TEM) are positively charged (zeta potential=+42.02 mV), which allowed for the subsequent self-assembly of a negatively charged PSS polyelectrolyte and a positively charged G5.NH₂-FI-FA or G5.NH₂-FI dendrimer. The Fe₃O₄/PSS/G5.NH₂-FI-FA or Fe₃O₄/PSS/G5.NH₂—FINPs formed were subjected to an acetylation reaction to neutralize terminal amine groups of the dendrimers (See FIG. 37). Zeta potential measurements were used to monitor each step of the coating and functionalization of Fe₃O₄ NPs (See Table 2). The alternating charge reversal of Fe₃O₄ NPs after coating with PSS and G5.NH₂-FI or G5.NH₂-FI-FA dendrimers indicated successful electrostatic assembly.

TABLE 2
Zeta potential values of Fe₃O₄NPs after each step modification.

| Nanoparticles | Zeta potential (mV) |
|---|---|
| Fe₃O₄ | +42.02 |
| Fe₃O₄/PSS | −45.04 |
| Fe₃O₄/PSS/G5.NH₂-FI | +52.81 |
| Fe₃O₄/PSS/G5.NH₂-FI-FA | +43.08 |
| Fe₃O₄/PSS/G5.NHAc-FI | +31.48 |
| Fe₃O₄/PSS/G5.NHAc-FI-FA | +23.86 |

After acetylation reaction, the zeta potentials of both Fe₃O₄/PSS/G5.NHAc-FI and Fe₃O₄/PSS/G5.NHAc-FI-FA significantly decreased due to the conversion of dendrimer surface amine groups to acetamide groups. The zeta potentials of neither Fe₃O₄/PSS/G5.NHAc-FI nor Fe₃O₄/PSS/G5.NHAc-FI-FA NPs were close to zero something not observed with the zeta potentials of dendrimer-encapsulated gold NPs. This implies that some of the dendrimer terminal amines that tangled with PSS polymer chains due to electrostatic interaction cannot be acetylated. Although an understanding of the mechanism is not necessary to practice the present invention and the present invention is not limited to any particular mechanism of action, in some embodiments, the less positive charges of Fe₃O₄ NPs does not induce significant non-specific binding with tumor cells, because the outermost side of dendrimer surface amines are acetylated. The formed functionalized Fe₃O₄ NPs with bilayer coating and acetylation reaction are colloidally stable in aqueous solution for at least 6 months at a concentration up to 20 mg/mL.

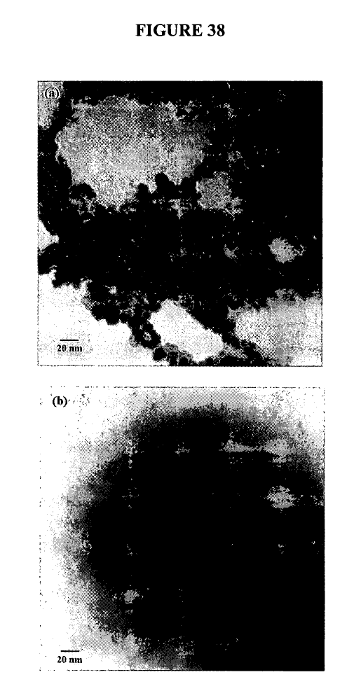

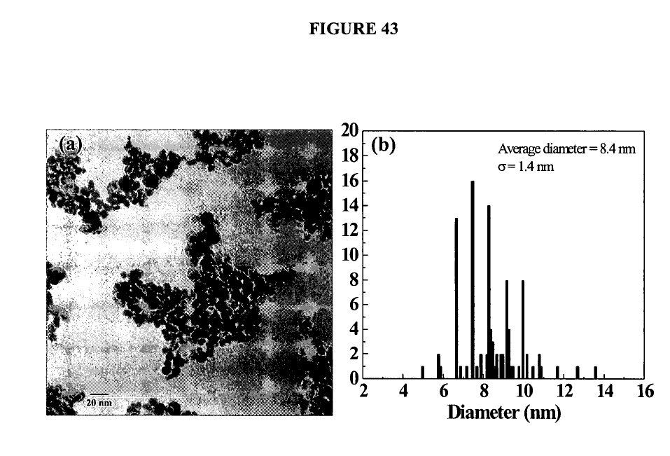

The self-assembled bilayers of PSS/G5.NHAc-FI and PSS/G5.NHAc-FI-FA on Fe₃O₄ NPs were also characterized by TEM imaging. The TEM image of PSS/G5.NHAc-FI-FA-coated Fe₃O₄ NPs (FIG. 38a) shows that, after bilayer self-assembly and chemical functionalization, the particles display similar morphology to the ones before self-assembly (FIG. 43). A negatively stained (with phosphotungstic acid) TEM image (FIG. 38b) shows that all Fe₃O₄ NPs are surrounded with bright rings of the polymer bilayers of PSS/G5.NHAc-FI-FA, further confirming the successful self-assembly process. PSS/G5.NHAc-FI-coated Fe₃O₄ NPs display similar polymer ring structures to those of PSS/G5.NHAc-FI-FA-coated Fe₃O₄ NPs as observed from negatively stained TEM images.

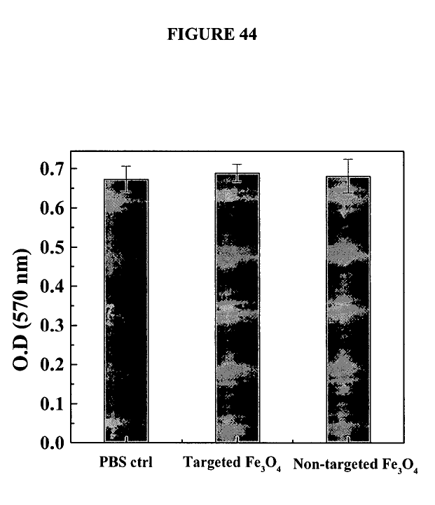

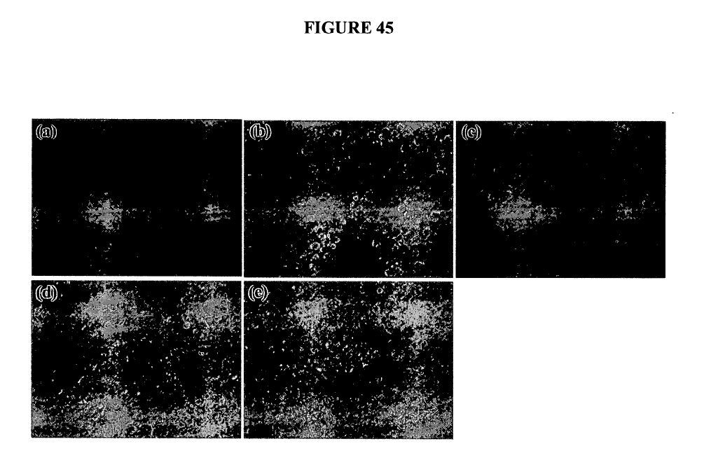

Cytotoxicity of the functionalized Fe₃O₄ NPs was evaluated by an MTT assay and by observing cell morphology changes after incubation with Fe₃O₄ NPs for 96 h. MTT assay data show that the functionalized Fe₃O₄ NPs with or without FA conjugation do not display cytotoxicity to KB cells (a human epithelial carcinoma cell line) at an Fe concentration of 45 μg/mL (See FIG. 44). Phase contrast microscopy images show that even at an Fe concentration of up to 360 μg/mL, KB cells treated with both Fe₃O₄/PSS/G5.NHAc-FI and Fe₃O₄/PSS/G5.NHAc-FI-FA NPs display the same morphology as those treated with PBS buffer (See FIG. 45), indicating that the PSS/dendrimer bilayer functionalized Fe₃O₄ NPs are biocompatible at an Fe concentration of up to 360 μg/mL.

The FA and the dye FI modified onto the G5 dendrimer surface were used as a targeting ligand and an imaging molecule, respectively. This affords the functionalized Fe₃O₄ NPs with both targeting and imaging functionalities. Folic acid receptor (FAR) is well known to be overexpressed in several human carcinomas including breast, ovary, endometrium, kidney, lung, head and neck, brain, and myeloid cancers (See, e.g., Weitman, D. et al. Cancer Res. 52, 3396-3401 (1992); Campbell et al., Cancer Res. 51, 5329-5338 (1991); and Ross et al., Cancer Res. 73, 2432-2443 (1994)). In this study, KB cells expressing both high- and low-level FAR (denoted as KB-HFAR and KB-LFAR, respectively) were selected for the intracellular uptake of functionalized Fe₃O₄ NPs.

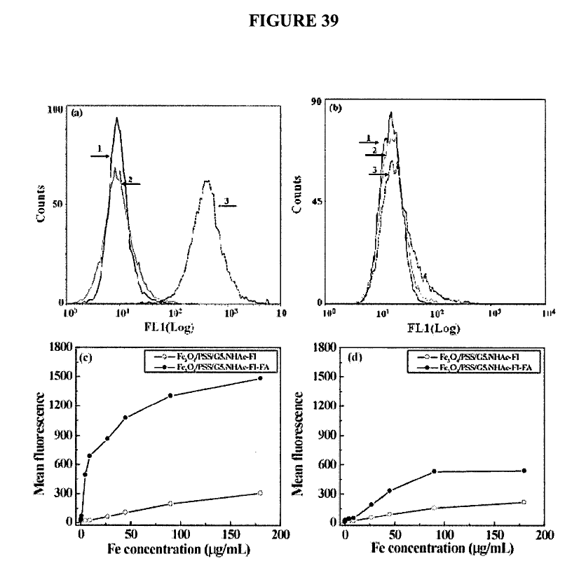

FIG. 39 illustrates the binding of both PSS/G5.NHAc-FI-FA- and PSS/G5.NHAc-FI-coated Fe₃O₄ NPs (Fe concentration=9 μg/mL) with KB-HFAR and KB-LFAR cells investigated by flow cytometry. It is clear that after binding of PSS/G5.NHAc-FI-FA-coated Fe₃O₄ NPs with KB-HFAR cells, the fluorescence signal significantly increases (See FIG. 39a). In contrast, PSS/G5.NHAc-FI-modified Fe₃O₄ NPs without FA conjugation display a similar fluorescence signal to the PBS control, suggesting no measurable binding with KB-HFAR cells. For KB-LFAR cells, neither Fe₃O₄/PSS/G5.NHAc-FI nor Fe₃O₄/PSS/G5.NHAc-FI-FA NPs displays significant binding (See FIG. 39b). Thus, high affinity binding of the FA-modified Fe₃O₄ NPs with KB cells is mediated by the FAR.

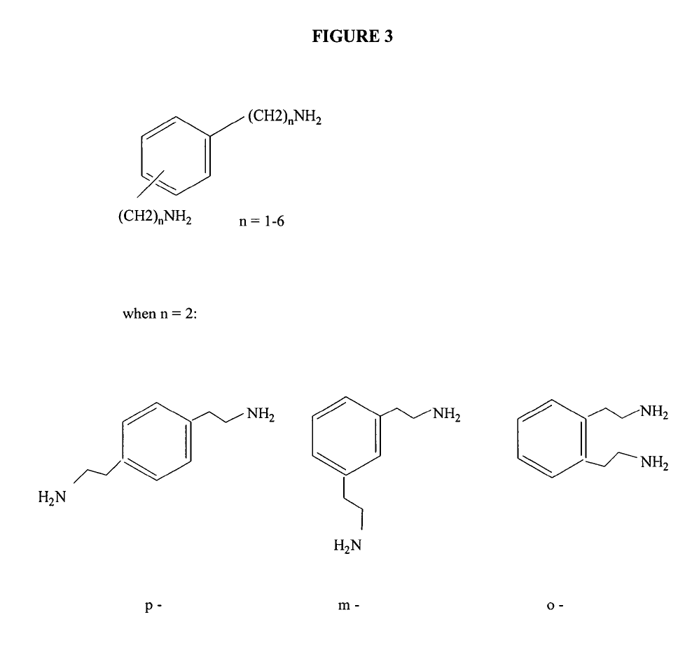

The dose-dependent cellular uptake of the functionalized Fe₃O₄ NPs (FIGS. 3c and 3d). was also analyzed. At an Fe concentration above 4.5 μg/mL, KB-HFAR cells treated with Fe₃O₄/PSS/G5.NHAc-FI-FA NPs show remarkably higher fluorescence signals than those treated with Fe₃O₄/PSS/G5.NHAc-FI NPs without FA conjugation (See FIG. 39c), indicating the high affinity of FAR-mediated specific uptake.

For KB-LFAR cells, both Fe₃O₄/PSS/G5.NHAc-FI and Fe₃O₄/PSS/G5.NHAc-FI-FA NPs display much less uptake into cells than those incubated with KB-HFAR cells, even at an Fe concentration of up to 180 μg/mL (See FIG. 39d). However, in the studied concentration range, Fe₃O₄ NPs with FA modification exhibit more uptake in KB-LFAR cells than those without FA modification, which is quite different than that of single FA-modified G5 dendrimers (See, e.g., Thomas, T. P. et al. J. Med. Chem. 48, 3729-3735 (2005)). Thus, the present invention demonstrates that the FA-modified Fe₃O₄ NPs display higher binding sensitivity than that of FA-modified G5 dendrimers. Although an understanding of the mechanism is not necessary to practice the present invention and the present invention is not limited to any particular mechanism of action, in some embodiments, the higher binding capacity of FA-modified Fe₃O₄ NPs may stem from polyvalency effects due to multiple FA ligands presented on each Fe₃O₄ NP surface (See, e.g., Quinti, et al., Nano Lett. 6, 488-490 (2006); Zhao et al., Bioconjugate Chem. 13, 840-844 (2002)).

The number of FA ligands (n) per Fe₃O₄ NP can be calculated according to the following equation:

$$n = {n}_{1} \times \frac{4 π {r}_{1}^{2}}{π {r}_{2}^{2}} , \qquad ( 1 )$$

where r₁ and r₂ are the radius of PSS-modified Fe₃O₄ NPs and G5.NHAc-FI-FA dendrimers, respectively, and n₁ is the number of FA moieties per G5 dendrimer. Note that the calculation is based on the following assumptions: (1) a densely packed monolayer of G5.NHAc-FI-FA dendrimer is presented onto the Fe₃O₄ NP surfaces; (2) each dendrimer molecule shows a pancake shape when deposited onto the Fe₃O₄ NP surfaces (See, e.g., Bosman et al., Chem. Rev. 99, 1665-1688 (1999); Imae, T. et al., Langmuir 15, 4076-4084 (1999)) and the diameter of the pancake shape does not change significantly, compared with that of dendrimers in solution; (3) the PSS polymer layer thickness is 2 nm (See, e.g., Caruso et al., Macromolecules 32, 2317-2328 (1999)); and (4) there are half the number of FA (2.4) moieties presented in each dendrimer molecule available for binding (based on the geometry of dendrimer shape and stochastic distribution of FA moieties onto each G5.NHAc-FI-FA dendrimer). The number of FA moieties per Fe₃O₄ NPs was calculated to be ˜35.6, using the average diameter of PSS-coated Fe₃O₄ NPs (10.4 nm) and G5 dendrimers (5.4 nm) (See, e.g., Tomalia et al., Angew. Chem. Int. Ed. Engl. 29, 138 (1990)). The larger number of FA moieties per Fe₃O₄ NP compared with single FA-modified dendrimer (4.8 FA per dendrimer) facilitates the polyvalency effect, thereby significantly increasing the binding sensitivity of Fe₃O₄ NPs with KB cells through FAR mediation.

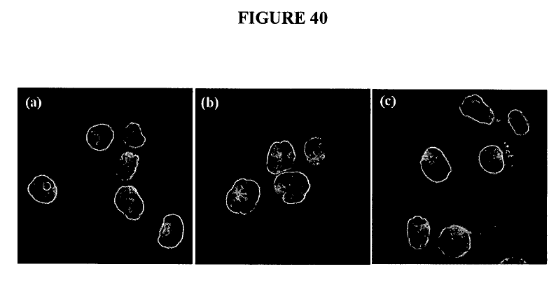

The self-assembly of G5.NHAc-FI-FA dendrimers onto Fe₃O₄ NPs also affords the utilization of confocal microscopic imaging of the intracellular uptake of Fe₃O₄ NPs. It is clear that after treatment with Fe₃O₄/PSS/G5.NHAc-FI-FA NPs for 2 h, the green FI fluorescence signals appeared in the cytosol of KB-HFAR cells (See FIG. 40c). In contrast, the same KB-HFAR cells treated with Fe₃O₄/PSS/G5.NHAc-FI without FA modification do not show any FI fluorescence signal, which is similar to the KB-HFAR cells treated with PBS buffer (FIGS. 40a and 40b). Thus, the confocal imaging data provides that the intracellular uptake of Fe₃O₄/PSS/G5.NHAc-FI-FA NPs into KB-HFAR cells is through FAR-mediated endocytosis.

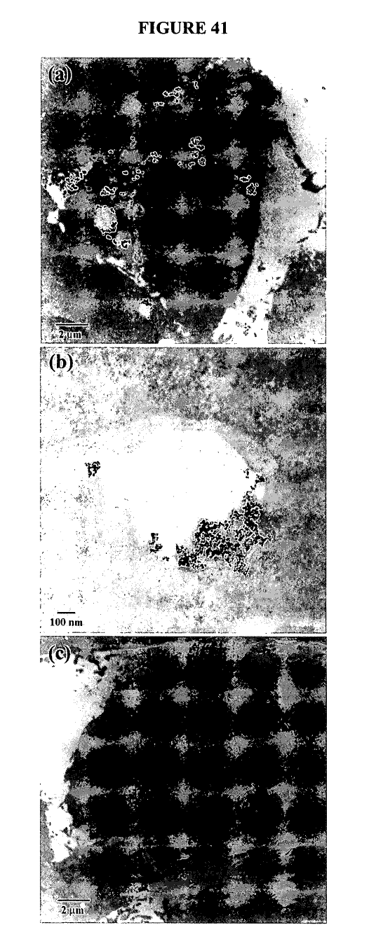

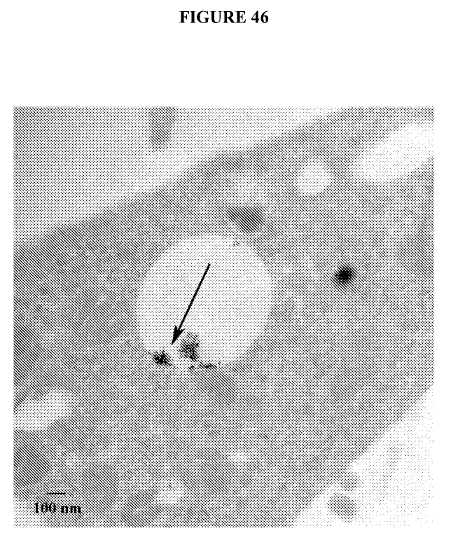

The specific intracellular uptake of FA-modified Fe₃O₄ NPs was further verified by TEM. The TEM imaging technique allows for clear identification of the Fe₃O₄ NPs in different cellular entities. TEM images of KB-HFAR cells treated with Fe₃O₄/PSS/G5.NHAc-FI-FA NPs for 2 h show that the NPs distributed predominantly into the vacuoles of the cells (See FIGS. 41a and 41b). Uptake of Fe₃O₄/PSS/G5.NHAc-FI-FANPs in the lyosomes and the nucleus was not observed. Significant uptake of Fe₃O₄/PSS/G5.NHAc-FI NPs without FA modification was not observed (See FIG. 41c). There was only a minimal uptake of Fe₃O₄/PSS/G5.NHAc-FI NPs randomly distributed in the vacuoles of some cells (See FIG. 46), which was undetectable using confocal microscopy. This minimal uptake might be related to diffusion-driven non-specific binding since control cells without treatment of Fe₃O₄ NPs did not show any internalized NPs. The TEM studies underscore the high specificity of FA-modified Fe₃O₄ NPs for targeting KB-HFAR cells, in agreement with the confocal imaging data. The specific uptake of Fe₃O₄/PSS/G5.NHAc-FI-FANPs is quite different from the targeted intracellular uptake of functionalized dendrimer-encapsulated gold NPs. In the latter case, the FA-functionalized gold NPs (3.2 nm in diameter) are distributed predominantly into lysosomes of the targeted cells within 2 h of incubation.

MR imaging is often used for diagnosis and staging of cancer. Iron oxide NPs affect the MR signal by dephasing transverse magnetization and hence reducing the value of T2. A targeted iron oxide NP would have a major benefit in cancer management by specifically detecting tumors that over-express the FA binding protein. To study the effect of Fe₃O₄/PSS/G5.NHAc-FI-FANPs on cancer cells, the T2 of KB-HFAR cells exposed to differing concentrations of Fe₃O₄/PSS/G5.NHAc-FI-FA NPs was measured. The T2 values of KB-HFAR cell pellets treated with Fe₃O₄/PSS/G5.NHAc-FI-FANPs dramatically decreased as a function of Fe concentration (See Table 3).

TABLE 3
MR signals of KB cells treated with functionalized Fe₃O₄NPs

| Concen- | | | | |
|---|---|---|---|---|
| tration | T1 (s) | | T2 (s) | |
| (μg/mL) | Fe₃O₄-FI^{a} | Fe₃O₄-FI-FA^{b} | Fe₃O₄-FI | Fe₃O₄-FI-FA |
| 0^{c} | 2.01 ± 0.07 | 2.01 ± 0.07 | 0.921 ± 0.05 | 0.921 ± 0.05 |
| 22.5 | 2.24 ± 0.07 | 1.58 ± 0.02 | 0.357 ± 0.002 | 0.332 ± 0.04 |
| 45 | 2.26 ± 0.03 | 1.66 ± 0.04 | 0.230 ± 0.008 | 0.106 ± 0.004 |
| 90 | 2.46 ± 0.07 | 1.23 ± 0.016 | 0.12 ± 0.005 | 0.042 ± 0.007 |

^{a}denotes Fe₃O₄/PSS/G5.NHAc-FI NPs.
^{b}denotes Fe₃O₄/PSS/G5.NHAc-FI-FA NPs.
^{c}PBS buffer.

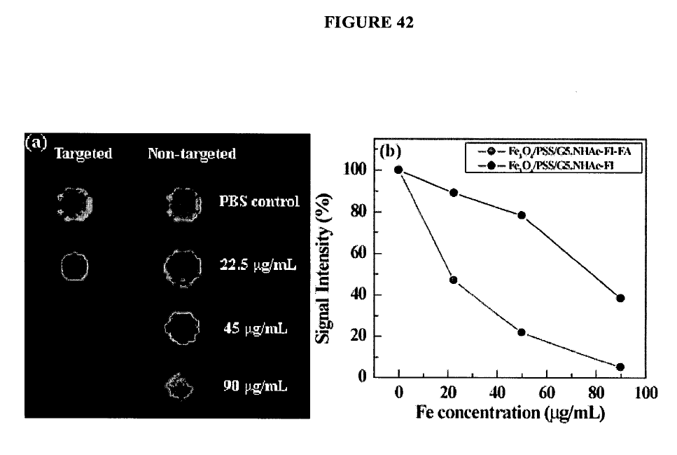

In contrast, the decreasing trend of T2 values as a function of Fe concentration for the same KB cells treated with Fe₃O₄/PSS/G5.NHAc-FI NPs is significantly less than Fe₃O₄/PSS/G5.NHAc-FI-FANPs. In the T2-weighted spin-echo MR images under Fe concentrations of 22.5 μg/mL, Fe₃O₄/PSS/G5.NHAc-FI-FANPs reduces the signal intensity to 50% of the initial value (PBS control) whereas Fe₃O₄/PSS/G5.NHAc-FINPs reduces the signal to about 89% of the initial value (See FIG. 42a). This provides that Fe₃O₄/PSS/G5.NHAc-FI-FA NPs can specifically hamper the MR signal through FAR-mediated binding and endocytosis. At higher Fe concentrations (e.g., 90 μg/mL), non-specific binding of Fe₃O₄/PSS/G5.NHAc-FI NPs without FA conjugation occurred with KB-HFAR cells. The dose-dependent quantitative MR signal intensity shown in FIG. 42b also provides support for the non-specific uptake of Fe₃O₄/PSS/G5.NHAc-FI NPs at higher Fe concentrations.

### Example 19

### Generation of Dendrimer-Functionalized Shell-Crosslinked Iron Oxide Nanoparticles and In Vivo Imaging of Tumors Using the Same

Materials and Methods.

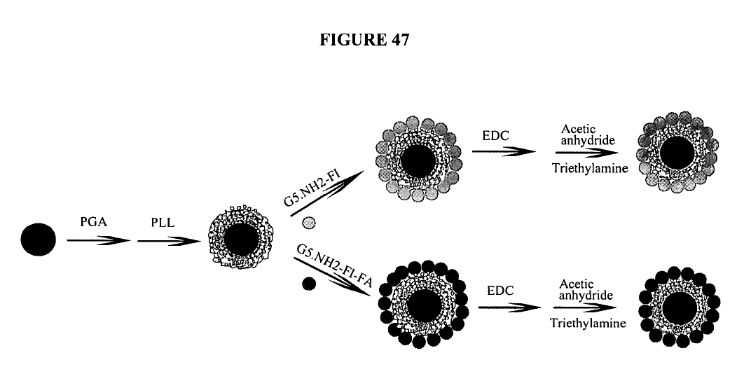

Fabrication of Multifunctional Shell-Crosslinked Iron Oxide (SCIO) Nanoparticles (NPs). The procedure used to fabricate multifunctional SCIO NPs is shown in FIG. 47. The layer-by-layer (LbL) assembly of oppositely charged poly(glutamic acid) (PGA) (Mw=15,000˜50,000, SIGMA) and poly-L-lysine (PLL) (Mw=15,000˜30,000, SIGMA) was performed as described (See, e.g., Schneider, G. et al. Nano Lett. 6, 530-536 (2006)). Briefly, a solution of Fe₃O₄ NPs (5 mg in 0.5 mL water, diameter 8.4±1.4 nm, synthesized and characterized as described in Example 18) was added with 1 mL of a PGA solution (1 mg/mL, pH 7.4 PBS buffer containing 0.5 M NaCl) with occasional shaking. After adsorption of PGA for 20 min, the suspension was centrifuged at 8,000 rpm for 10 min. The supernatant was then carefully removed, and the coated Fe₃O₄ NPs were washed by three alternate cycles of centrifuging and resuspending the particles in pure water. Then 1 mL of PLL solution (1 mg/mL, pH 7.4 PBS buffer containing 0.5 M NaCl) was added into the PGA-modified Fe₃O₄ NP suspension and purified in the same manner. These steps were repeated until 5 layers of (PGA/PLL)₂PGA deposited onto the Fe₃O₄ NPs. Then, the outermost layer of FI- and FA-functionalized generation 5 PAMAM dendrimers (G5.NH₂-FI-FA) (synthesized and characterized as described in Example 18, solution prepared at a concentration of 1 mg/mL in pH 7.4 PBS buffer containing 0.5 M NaCl) was deposited in the same way and the final (PGA/PLL)₂PGA/G5.NH₂-FI-FA-modified Fe₃O₄ NPs were dispersed into 50 mM MES buffer (pH=5.5, ALDRICH) and EDC (12-18 mg, ALDRICH) was added to crosslink the hydroxyl groups of Fe₃O₄ NPs and the amino groups of PLL and dendrimers with the carboxyl groups of PGA. The mixture was shaken overnight, followed by 3 cycles of centrifugation/redispersion (in water) steps to remove unreacted reactants. The SCIO NPs with FA modification (SCIO-FA NPs) were subjected to an acetylation reaction to neutralize the remaining amine groups of G5.NH₂-FI-FA dendrimers. In brief, the NPs (in 1 mL water) were added with triethylamine (5.0 μL, ALDRICH) and mixed well. Then, a methanol solution (0.1 mL) containing 3.64 mg acetic anhydride (ALDRICH) was added dropwise into the NP/triethylamine solution. The reaction mixture was vigorously shaken for 24 h. The formed neutralized SCIO-FA NPs were purified by 4 cycles of centrifugation/washing/redispersion in water. For biological testing, the NPs were transferred to a PBS buffer solution by centrifugation and redispersion. The NPs were stored at 4° C. before biological testing. The control SCIO NPs (Fe₃O₄/(PGA/PLL)₂/PGA/G5.NHAc-FI NPs) without FA conjugation (SCIO-NonFA NPs) were prepared in the same manner as the procedure used to prepare SCIO-FA NPs. The FI-modified amine-terminated G5 dendrimers (G5.NH₂—FI) used were prepared and characterized as described in Example 18.

General Characterization Methods. FTIR spectra were acquired using a PERKIN ELMER SPECTRUM GX FTIR system. Dry particles were mixed with milled KBr crystals and the samples were pressed as pellets before measurements. The iron concentration of Fe₃O₄ NPs before and after surface modification was determined by a PERKIN ELMER OPTIMA 2000 DV Inductively Coupled Plasma-Optical Emission Spectroscopy (ICP-OES). The surface potential of functionalized Fe₃O₄ NPs was measured by a Malvern ZETASIZER NANO ZS model ZEN3600 (Worcestershire, UK) equipped with a standard 633 nm laser. The size and morphology of the Fe₃O₄ NPs were characterized by a Philips CM-100 TEM equipped with a Hamamatsu Digital Camera ORCA-HR operated using AMT software (Advanced Microscopy Techniques Corp, Danver, Mass.). The operation voltage was kept at 60 kV. TEM samples were prepared by deposition of a diluted particle suspension (5 μL) onto a carbon-coated copper grid and were air-dried before the measurement. Stained specimens were prepared by depositing the sample solutions on the grid and inverting the grid on a drop of aqueous phosphotungstic acid solution that had been neutralized with NaOH (2% mass fraction of the phosphotungstic acid). The grid was then blotted on filter paper and air-dried. In order to investigate the morphology of the polymer hollow capsules before and after EDC crosslinking, the SCIO NPs were exposed to 3 M HCl to erode the Fe₃O₄ core particles. The formed iron salt in the solution was removed and washed with water by membrane filtration through a 10,000 MWCO membrane using MILLIPORE CENTRICON devices. The concentrated aqueous solutions of hollow polymer capsules were applied for TEM imaging as described above. MR relaxometry of SCIO NPs was performed using a 2.0 T Varian Unity/Inova system (Palo Alto, Calif.) using home-built RF coils. SCIO NPs were diluted in water at variable concentrations. For MR relaxometry measurements, 1 mL SCIO NPs were filled in 1.5 mL Eppendorf vials. T₂ relaxation times were measured using a standard Carr-Purcell-Meiboom-Gill pulse sequence (TR=2000 ms, TE range 30-960 ms, 32 echoes, FOV=134×67 mm, matrix 128×64, slice thickness 10 mm, BW=40, NEX=3). T₂ relaxation times were calculated by a linear fit of the logarithmic ROI signal amplitudes versus TE. The T₂ relaxivities (r2) were determined by a linear fit of the inverse relaxation times as a function of the Fe concentration used.

KB Cell Culture. The KB cells (American Type Tissue Collection, Rockville, Md.) were continuously grown in two 24-well plates, one in FA-free medium and the other in regular RPMI 1640 medium (GIBCO-BRL, Gaithersburg, Md.) supplemented with penicillin (100 units/mL, SIGMA), streptomycin (100 μg/mL, Sigma), 10% heat-inactivated fetal bovine calf serum (FBS) (SIGMA), and 2.5 μM FA. The cells grown in FA-free medium express high-level FAR, while the cells grown in FA-containing medium express low-level FAR.

Determination of Cell Viability. Cell viability was measured by fluorescein diacetate (FDA, SIGMA) and propidium iodide (PI, SIGMA) staining. FDA stains live cells, while PI stains dead cells. The stained cells were quantified by flow cytometry as described by Killinger (See, e.g., Killinger, et al., Ann. Thorac. Surg. 53, 472-476 (1992)). Briefly, 2×10⁵ KB cells per well were seeded into a 24-well plate and incubated with 0-100 μg/ml of unmodified Fe₃O₄ NPs, SCIO-NonFA NPs, and SCIO-FA NPs for 24 h at 37° C. Ten thousand cells were acquired from each sample for flow cytometric analysis.

Determination of Binding Affinity by Flow Cytometry. Approximately 2×10⁵ cells per well were seeded in 24-well plates the day before the experiments. An hour before initiating an experiment, the cells were rinsed three times with serum-free and FA-deficient RPMI 1640 medium. SCIO-NonFA and SCIO-FA NPs were added at Fe concentrations of 0-50 μg/mL. After 1 h of incubation at 37° C., KB cells with both high- and low-level FAR were trypsinized (Trypsin-EDTA, GIBCO-BRL, Gaithersburg, Md.) and suspended in PBS (Dulbecco's PBS, GIBCO-BRL, Gaithersburg, Md.) containing 0.1% bovine serum albumin (GIBCO-BRL, Gaithersburg, Md.), and then analyzed using a Coulter EPICS-XL MCL BECKMAN-COULTER flow cytometer. The FL1-fluorescence of 10,000 cells was measured, and the mean fluorescence of gated viable cells was quantified using EXPO32 software (BECKMAN-COULTER, Miami, Fla.).

In Vitro MR Relaxometry and Imaging. 5×10⁶ KB-HFAR cells were incubated with SCIO-NonFA and SCIO-FANPs with Fe concentrations of 6.3, 12.5, and 25 μg/mL for 30 min in an ice bath. Live cells are usually cultured with a complete medium at 37° C. For the MRI studies, live cells were trypsinized and suspended in PBS (instead of cell culture medium) and incubated with NPs. Live cells in PBS have a higher viability at 4° C. or in an ice bath than at room temperature, so we incubated cells with NPs in an ice bath. The cells were then washed with PBS buffer three times. The cells were centrifuged to prepare pellets for MRI. Studies were performed with a 2.0 T Varian Unity/Inova system (Palo Alto, Calif.) using home-built RF coils. One hundred microliters of PBS was added to each cell pellet, and the cells were suspended by gentle shaking. The T₁ and T₂ of the cell suspensions were measured in each sample vial with inversion recovery and CPMG pulse sequences, respectively. The cells were then allowed to settle and a phantom was constructed consisting of all of the sample vials. A spin-echo image (TR/TE 2000/8 ms) with a 2.0 mm-slice thickness and an in-plane resolution of 0.312 mm was acquired through the plane of the cells.

Tumor Model. A murine tumor model was established in NOD C.B-17 SCID mice using human KB tumor cells over-expressing folate receptor as described (See, e.g., Kukowska-Latallo, J. F. et al. Cancer Res. 65, 5317-5324 (2005)). Briefly, five- to six-week-old FOX CHASE SCID (C.B-17/lcrCrl-scidBR) female mice were purchased from the Charles River Laboratories (Wilmington, Mass.) and housed in a specific pathogen-free animal facility at the University of Michigan Health System in accordance with the regulations of the University's Committee on the Use and Care of Animals (UCUCA) as well as with federal guidelines, including the Principles of Laboratory Animal Care. Animals were fed ad libitum with Laboratory Autoclavable Rodent Diet 5010 (PMI Nutrition International, St. Louis, Mo.). The food was changed to a folate-deficient diet (TESTDIET, Richmond, Ind.) for 7 days prior to injection of tumor KB cells. The KB cell suspension of 1×10⁶ was injected subcutaneously into both flanks of each mouse in the same time. When the tumor nodules had reached a volume of 0.60±0.15 cm³ (approximately 3 weeks post-injection), the animals were randomly allocated into control, SCIO-NonFA, and SCIO-FA groups. SCIO-FA and SCIO-NonFA NPs were delivered via the tail vein in 0.1 mL of saline at 12.4 μg Fe/per mouse, respectively. Two-dimensional and three-dimensional MRI images were obtained both before and after administration of either imaging agent at time points of hours 1, 4, 8, 24, 48, and day 7 after injection.

Anesthesia for imaging was induced by placing mice in a chamber with 2.0% isofluoroane (SBH Scientific). Following anesthetic induction, the mice were transferred to the MRI probe and anesthesia was maintained at 1.25% isoflurane for the duration of the study. All procedures were performed under protocol approval by UCUCA at the University of Michigan.

In Vivo MR Imaging. The MRI probe, constructed specifically for these studies, was based on an Alderman-Grant slotted cylinder design (length 10 cm, OD 4.5 cm). The probe was made with polycarbonate tubing, copper tape, and ATC and Johanson capacitors. Following induced anesthesia, the mouse to be imaged was placed inside a second polycarbonate tube (ID 2.6 cm). This second tube was then inserted into the MR probe, allowing easy animal positioning and restricting the mouse MRI studies to a region of homogeneous RF field. MRI was performed on a 2T Varian Unity/Inova system equipped with Acustar S180 gradients.

At each time point for each animal, 2D and 3D gradient-echo MRI images were obtained. Two sets of interleaved, 2D gradient-echo images were acquired with a 2 mm slice thickness, TR/TE 100/5 ms, flip angle 45°, in plane resolution 390 mm, and 8 averages. The total time to acquire the 2D images was 2.5 min. The 3D gradient-echo images were acquired with a TR/TE of 20/4 ms, a flip angle of 20°, isotropic voxel resolution of 390 mm, and 4 averages. Imaging time for the 3D dataset was 5.5 min. The 3D gradient echo pulse sequence was chosen to provide isotropic spatial resolution, minimize motion artifacts, and generate T₂ weighted MR images.

All image data was transferred to a remote computer (Dual 2 GHz PowerPC G5 Macintosh) for data processing. MATLAB (The MATHWORKS, Natick, Mass.) was used to convert the k-space MR data sets into quantitative MR images and to combine all images of the study into a single dataset. The tumor was typically present in 30 of 64 planes in the 3D image. The entire tumor was selected from the dataset and used to generate normalized histograms of the signal intensity. Using the data from 3D MRI studies assures that the entire tissue of interest is examined and that no biasing toward anomalously enhancing regions occurs. The percent enhancement was calculated for each of the images acquired at each time following injection of either the targeted or the non-targeted SCIO agent. Histograms in the figures are plotted as the percentage of voxels at a given percent enhancement.

Generation of multifunctional iron oxide NPs using shell-crosslinked polymer multilayers and functional characterization of the same.

As described in Example 18, Fe₃O₄ NPs modified through an approach that combines a layer-by-layer (LbL) self-assembly technique and dendrimer chemistry can specifically target to tumors cells overexpressing folic acid receptor (FAR) in vitro. For example, a bilayer composed of polystyrene sulfonate sodium salt (PSS) and FA- and FI (fluorescein isothiocyanate)-functionalized poly(amidoamine) (PAMAM) dendrimers of generation 5 (G5.NH₂-FI-FA) could be assembled onto Fe₃O₄ NPs through electrostatic LbL assembly, followed by acetylation of the remaining surface amine groups of the assembled G5 dendrimers, that were able to target tumor cells expressing FAR in vitro. Unfortunately, in vivo data generated using the NPs demonstrated that most of the bilayer-modified Fe₃O₄ NPs accumulated in the liver of mice, suggesting that the particles lacked in vivo stability. Thus, experiments were conducted to determine whether multifunctional dendrimers (e.g., comprising targeting, imaging and/or therapeutic moieties) could be assembled with iron oxide NPs in such a way so as to be biologically useful (e.g., for successful in vivo MRI of a tumor).

As described in the materials and methods above, iron oxide NPs were assembled with poly(glutamic acid) (PGA) and poly-L-lysine (PLL), followed by assembly with G5.NH₂-FI-FA dendrimers. Then, the interlayers were crosslinked using EDC chemistry to covalently link the hydroxyl groups of iron oxide, the carboxyl groups of PGA, and the amino groups of PLL and the dendrimers. The remaining amino groups of dendrimers were acetylated to neutralize the surface charge (See FIG. 47). The formed shell-crosslinked iron oxide (SCIO) NPs were characterized using the zeta potential, Fourier transform infrared (FTIR) spectroscopy, transmission electron microscopy (TEM), and relaxivity measurements. As described herein, combined flow cytometry and in vitro and in vivo MRI studies show that the FA-modified SCIO NPs can specifically bind to tumor cells and an early-stage tumor model overexpressing FAR.

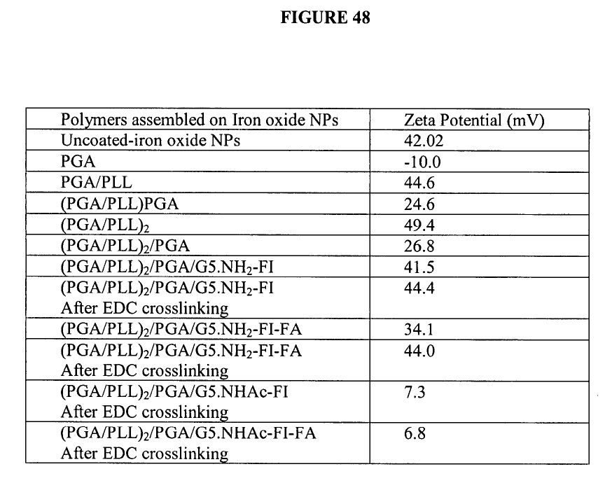

LbL self-assembly, shell crosslinking, and chemical modification of the SCIO NPs were monitored by zeta-potential measurement (See FIG. 48). The positively charged Fe₃O₄ NPs change to negative charge after PGA modification. After PLL assembly, the Fe₃O₄ NPs again reverse to positive charge. Further PGA modification decreases the surface charge. The changes in charge of the Fe₃O₄ NPs indicate the successful electrostatic assembly of (PGA/PLL)₂/PGA/G5.NH₂-FI-FA multilayers. The EDC crosslinking reaction does not result in a significant change of the surface charge of Fe₃O₄ NPs. However, after acetylation reaction, the zeta potentials of both (PGA/PLL)₂/PGA/G5.NHAc-FI and (PGA/PLL)₂/PGA/G5.NHAc-FI-FA NPs are close to neutral due to the conversion of dendrimer surface amine groups to acetamide groups. This is in sharp contrast to the acetylation of dendrimer/polystyrene sulphonate sodium salt bilayer-modified Fe₃O₄ NPs described in Example 18 that did not result in Fe₃O₄ NPs with close to a neutral charge. Although an understanding of the mechanism is not necessary to practice the present invention and the present invention is not limited to any particular mechanism of action, in some embodiments, this is due to the EDC crosslinking reaction that permits the dendrimer amines that have interacted with PGA polymer through electrostatic interaction covalently couple with PGA.

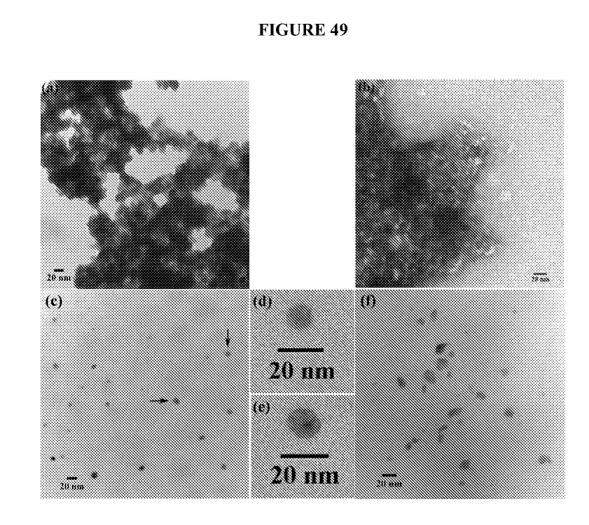

The fabricated SCIO NPs were characterized by TEM (See FIG. 49a-b). The morphology of the FA-modified SCIO (SCIO-FA) NPs does not show significant change after the assembly and crosslinking of the polymers and dendrimers when compared with the pristine Fe₃O₄ NPs described in Example 18 (See FIG. 49a). A negatively stained TEM image using phosphotungstic acid (See FIG. 49b) shows that all Fe₃O₄ NPs are surrounded with the bright rings of the polymer multilayers, confirming the successful self-assembly process. The non-targeted SCIO (SCIO-NonFA) NPs displayed a morphology similar to that of SCIO-FA NPs. The fabricated SCIO NPs are very stable both in aqueous solution and in cell culture medium for at least 6 months at Fe concentrations of up to 10 mg/mL.

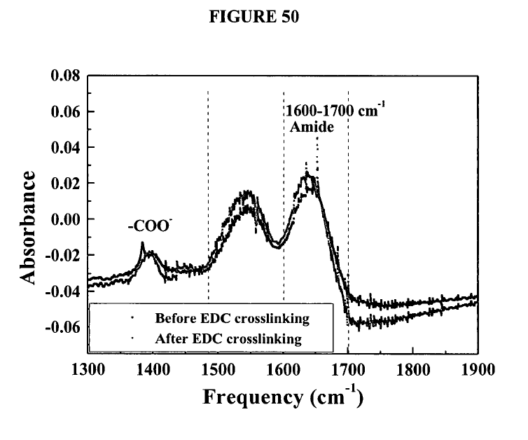

The EDC chemical crosslinking reaction was confirmed by FTIR spectrometry (See FIG. 50). The absorbance of the amide bond of (PGA/PLL)₂/PGA/G5.NH₂-FI-FA-modified Fe₃O₄ NPs increased after EDC crosslinking when compared to the same NPs before EDC crosslinking. Although FTIR is not a very effective characterization approach to characterize the intensity of the amide bond before and after shell crosslinking because the polymer PGA, PLL, and dendrimers that were used themselves also contain many amide bonds, the FTIR spectra qualitatively verify the formation of amide bonds between the carboxyl groups of PGA and the amine groups of PLL and dendrimers.

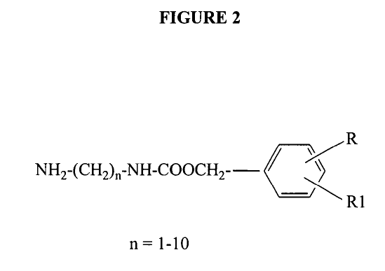

To further confirm the improvement of the mechanical stability after EDC shell crosslinking, the morphology of the polymer shells after the removal of the iron oxide core particles was investigated (See FIG. 49c-f). It was observed that before EDC crosslinking, intact hollow polymer nanocapsules can be formed (See FIG. 49c-e). Some of the capsules have a completely empty interior (See FIG. 49d), while some of the capsules still contain iron residues due to incomplete removal (See FIG. 49e). In sharp contrast, after EDC crosslinking, most of the capsules formed are broken (FIG. 2f). Although an understanding of the mechanism is not necessary to practice the present invention and the present invention is not limited to any particular mechanism of action, in some embodiments, this is due to the huge rupture force induced instantaneously by the swelling and dissolution of iron oxide cores, further confirming that the mechanical stability of the SCIO NPs is significantly improved, while the permeability of the capsules after EDC crosslinking is decreased. This further evidences the successful EDC crosslinking reaction.

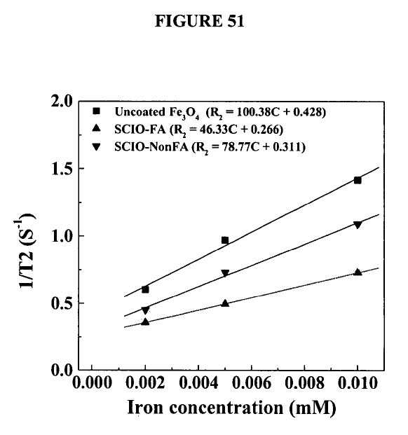

The transverse relaxation time (T₂) of water protons in aqueous solution of fabricated SCIO NPs was measured at 2 Tesla with a CPMG pulse sequence and the measured data were used to compute the transverse relativity (r₂) (the transverse relaxation rate per mM iron). The r₂ of uncoated Fe₃O₄ NPs, SCIO-FA NPs, and SCIO-NonFA NPs as a function of Fe concentration are shown in FIG. 51. The uncoated Fe₃O₄ NPs show the highest r₂ relativity (r2=100.4 s⁻¹ mM⁻¹), whereas the r₂ relaxivities of SCIO-FA and SCIO-NonFA NPs are somewhat reduced at 46.3 and 78.8 s⁻¹ mM⁻¹, respectively. The polymer coating onto the Fe₃O₄ NPs shields water molecules from their surfaces, and causes the lower r₂ relativity of SCIO NPs. Although an understanding of the mechanism is not necessary to practice the present invention and the present invention is not limited to any particular mechanism of action, in some embodiments, compared with SCIO-NonFA NPs, the presence of the FA moieties of SCIO-FA NPs may significantly increase the hydrophobicity and hindrance of the coating layers, thereby enhancing the shielding effect (See, e.g., Zhang, C. et al. Langmuir 23, 1427-1434 (2007)). As a consequence, SCIO-FA NPs exhibit lower r₂ relaxivity than that of SCIO NPs without FA.

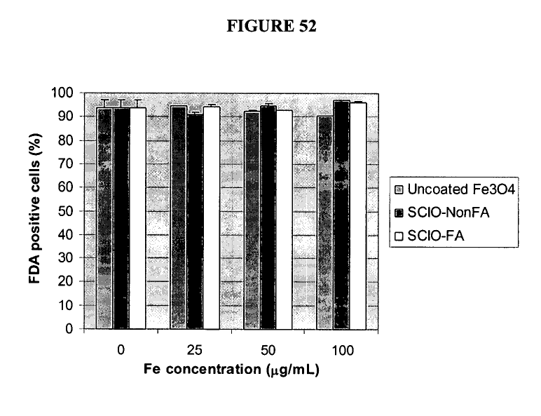

The cytotoxicity of the functionalized SCIO NPs was evaluated by fluorescein diacetate (FDA) and propidium iodide (PI) staining. Cell viability data (See FIG. 52) showed that the KB cells (a human epithelial carcinoma cell line) treated by SCIO NPs with or without FA conjugation display similar percentage of FDA positive cells to the KB cells treated by unmodified Fe₃O₄ NPs at an Fe concentration of 0-100 μg/mL. Thus, in some embodiments, the present invention provides that the SCIO NPs are biocompatible at an Fe concentration of 0-100 μg/mL, or more.

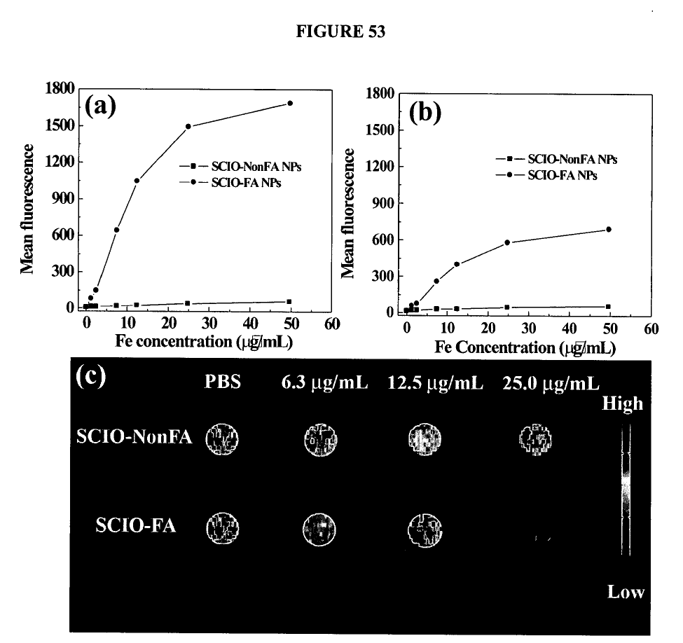

The FA and the dye FI modified onto the G5 dendrimer surfaces were used as targeting ligands and imaging molecules, respectively. The attached FI moieties afford a flow cytometric study of the binding of SCIO NPs with target cells. KB cells expressing both high and low levels of FAR (denoted as KB-HFAR and KB-LFAR, respectively) were selected to characterize the intracellular uptake of SCIO NPs. FIG. 53a-b illustrate the dose-dependent cellular uptake of the binding of SCIO-FA and SCIO-NonFA NPs. At an Fe concentration above 2.5 μg/mL, KB-HFAR cells exposed to SCIO-FA NPs show remarkably higher fluorescence signals than those treated with SCIO-NonFA NPs without FA (See FIG. 53a). With the increase of Fe concentration, the mean fluorescence levels off. Thus, the present invention provides that the high affinity of FAR mediates specific uptake of the NPs. Both SCIO-NonFA and SCIO-FA NPs display much less uptake in KB-LFAR cells than in KB-HFAR cells, even at an Fe concentration of up to 50 μg/mL (See FIG. 53b). However, in the studied concentration range, SCIO-FA NPs exhibit more uptake in KB-LFAR cells than SCIO-NonFA NPs without FA modification, which is similar to what was observed with these cells in Example 18. Although an understanding of the mechanism is not necessary to practice the present invention and the present invention is not limited to any particular mechanism of action, in some embodiments, this is due to the larger number of FA moieties per SCIO-FA NP compared with a single FA-modified dendrimer, which facilitates polyvalent binding, thereby significantly increasing the binding affinity of Fe₃O₄ NPs to each KB-LFAR cell.

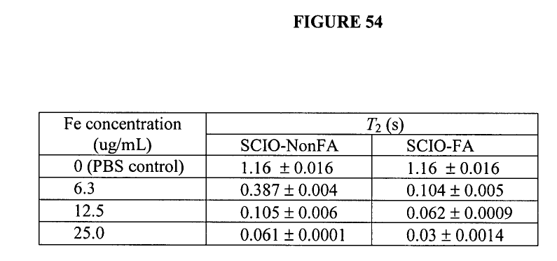

Iron oxide NPs affect the MRI signal by dephasing transverse magnetization and hence reducing the value of T₂. A targeted iron oxide NP would have a major benefit in tumor imaging by specifically detecting tumors that overexpress the FAR. To study the effect of the SCIO-FA NPs on tumor cells, the T₂ of KB-HFAR cells exposed to differing Fe concentrations of SCIO-FANPs was measured. The T₂ values of KB-HFAR cell pellets treated with SCIO-FANPs dramatically decreased as a function of Fe concentration (See FIG. 54). In contrast, the decreasing trend of T₂ values as a function of Fe concentration for the same KB cells treated with SCIO-NonFA NPs is significantly less than that of the KB cells treated with SCIO-FA NPs. In the T₂-weighted spin-echo magnetic resonance (MR) images (the color change from red to blue indicating the gradual decrease of MR signal intensity, which is similar to those reported based on the intensity of black color (See, e.g., Huh, Y.-M. et al., J. Am. Chem. Soc. 127, 12387-12391 (2005); Jun, Y.-W. et al. J. Am. Chem. Soc. 127, 5732-5733 (2005)) obtained using an Fe concentration of 25 μg/mL, SCIO-FANPs significantly diminished the signal (See FIG. 53c). Thus, the present invention provides that SCIO-FA NPs can specifically hamper the MR signal through FAR-mediated binding and endocytosis.

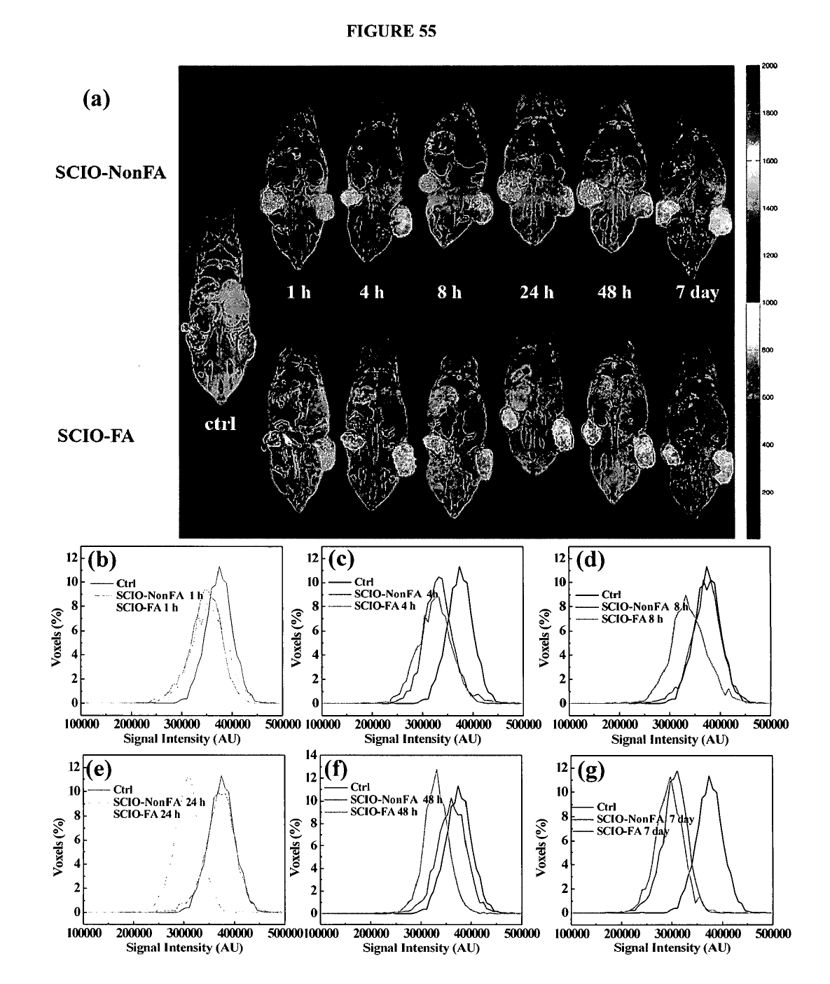

The in vivo MRI of tumors using SCIO NPs are presented on one slice of the 3-dimensional dataset from the images acquired following the injection of either targeted SCIO-FA or non-targeted SCIO-NonFA NPs (See FIG. 55a). The images were colorized to allow easy visualization of the contrast changes as a function of time post-injection. The control image was obtained from a mouse without treatment. The tumor MRI signal intensity of mice injected with SCIO-FA NPs gradually decreases as a function of time. In sharp contrast, the tumor MRI signal intensity of mice treated with SCIO-NonFA NPs does not decrease significantly with time post-injection. Twenty-four hours after injection of the SCIO-FA NPs, the tumor MRI signal intensity decreased more significantly than the signal intensity in the tumors of the mouse treated with non-targeted SCIO-NonFA NPs and in the control mouse. After 48 h post-injection, the difference of the MRI signal intensity of the tumors is smaller for both mice injected with SCIO-FA and SCIO-NonFA NPs. It is noted that the size of the colorized tumors shown in FIG. 55a may not be consistent because the T₂-weighted MR images of the mice may be taken at different positions and the images shown in FIG. 55a might not be in the same plane for the same mouse. MRI intensity data from whole tumor at different slices was collected and used to create the normalized statistical histograms of the signal decrease for all time points post-injection (See FIG. 55b-h). At the 24-h post-injection time point, the targeted SCIO-FA NP-treated tumor shows the most significant decrease of the signal intensity when compared with tumor treated with SCIO-NonFA NPs and the control mouse. After 48 h post-injection, the differences between the tumor MRI signal intensity of the targeted and non-targeted NPs becomes smaller.

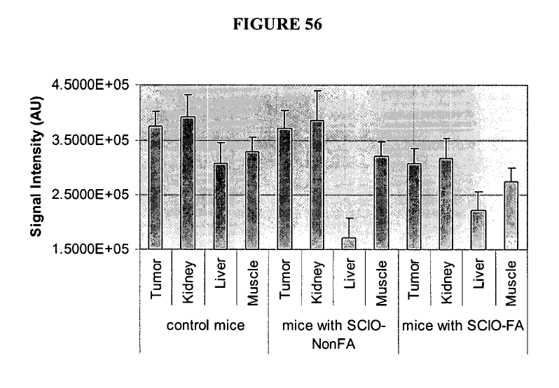

The differences in the MRI signal intensity of several major organs (such as the liver, kidney, muscle, and tumor) of different mice at 24 h after injection of the SCIO NPs were also compared in order to gain an understanding of the biodistribution of SCIO NPs (See FIG. 56). For SCIO-FA NP-treated mice, the MRI signal intensity of the tumor, kidney, and muscle decreased more significantly when compared with the control mice and the SCIO-NonFA NP-treated mice. Thus, the present invention provides that the SCIO-FA NP-treated mice show more iron oxide uptake in the three different tissues than the SCIO-NonFA NP-treated mice. However, the MRI signal intensity of the liver follows the order of: the control mice>SCIO-FA NP-treated mice>SCIO-NonFA NP-treated mice. Thus, the present invention provides that SCIO-NonFA NP-treated mice display more uptake of iron oxide in the liver than the SCIO-FA NP-treated mice.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

We claim:

1. A composition comprising dendrimers covalently linked to biopolymer-coated iron oxide nanoparticles.

2. The composition of claim 1, wherein said biopolymer-coated iron oxide nanoparticles are generated by a process comprising use of layer-by-layer self assembly of said biopolymer on said iron oxide nanoparticles.

3. The composition of claim 2, wherein said biopolymer comprises poly(glutamic acid) (PGA) and poly-L-Lysine (PLL).

4. The composition of claim 3, wherein the hydroxyl groups of said iron oxide nanoparticles, the carboxyl groups of PGA, the amino groups of said dendrimers and the amino groups of PLL are covalently linked.

5. The composition of claim 4, wherein the covalent attachments are formed by a process comprising crosslinking using 1-Ethyl-3-[3-dimethylaminopropyl]carbodiimide hydrochloride (EDC).

6. The composition of claim 5, wherein said composition is subjected to a surface neutralization reaction, wherein said surface neutralization reaction decreases the surface charge of said dendrimers.

7. The composition of claim 6, wherein said surface neutralization reaction comprises an acetylation reaction.

8. The composition of claim 7, wherein said acetylation reaction decreases the charge of unreacted amino groups of said dendrimers.

9. The composition of claim 1, wherein said dendrimer is a generation 5 (G5) polyamideamine (PAMAM) or polypropylamine (POPAM) dendrimer.

10. The composition of claim 1, wherein said biopolymer-coated iron oxide nanoparticle are about 8.4 nm in diameter.

11. The composition of claim 1, wherein said dendrimer further comprises one or more functional groups, wherein said one or more functional groups are selected from the group consisting of a therapeutic agent, a targeting agent, an imaging agent, and a biological monitoring agent.

12. The composition of claim 11, wherein said therapeutic agent is selected from the group consisting of a chemotherapeutic agent, an anti-oncogenic agent, an anti-angiogenic agent, a tumor suppressor agent, an anti-microbial agent, and an expression construct comprising a nucleic acid encoding a therapeutic protein.

13. The composition of claim 11, wherein said imaging agent comprises fluorescein isothiocyanate.

14. The composition of claim 11, wherein said targeting agent comprises folic acid.

15. A method of imaging a cancer cell comprising:
a) providing a composition comprising dendrimers covalently linked to biopolymer-coated iron oxide nanoparticles, wherein said dendrimers comprise a targeting agent that binds with specificity to said cancer cell; and
b) exposing said cancer cell to said composition under conditions such that said dendrimer interacts with and enters said cancer cell.

16. The method of claim 15, wherein said targeting agent is folic acid.

17. The method of claim 15, wherein said cancer cell expresses folic acid receptor.

18. The method of claim 15, wherein said cancer cell is present in vivo.

19. The method of claim 18, wherein said method images said cancer cell in a region beyond the primary tumor site.

20. The method of claim 19, wherein detection of said cancer cell in a region beyond the primary tumor site is indicative of metastasis.

21. The method of claim 15, wherein said imaging is used for staging of cancer.

22. The method of claim 15, wherein said dendrimer further comprises a therapeutic agent.

23. The method of claim 22, wherein said therapeutic agent is selected from the group consisting of a chemotherapeutic agent, an anti-oncogenic agent, an anti-angiogenic agent, a tumor suppressor agent, an anti-microbial agent, and an expression construct comprising a nucleic acid encoding a therapeutic protein.

24. The method of claim 15, wherein said dendrimer further comprises an imaging agent.

25. The method of claim 24, wherein said imaging agent comprises fluorescein isothiocyanate.

26. A kit comprising dendrimers covalently linked to biopolymer-coated iron oxide nanoparticles.

27. A method of generating a composition comprising dendrimers covalently linked to biopolymer-coated iron oxide nanoparticles comprising:
a) providing:
i) iron oxide nanoparticles;
ii) a pair of biocompatible polymers, wherein the first polymer comprises free amino groups and the second polymer comprises free carboxyl groups; and
iii) dendrimers; and
b) allowing layer-by-layer assembly of said first polymer, said second polymer and said dendrimers to occur on said nanoparticles;
c) crosslinking said layers, wherein said crosslinking comprises covalent attachment of hydroxyl groups of said nanoparticle, carboxyl groups of said second polymer, amino groups of said first polymer and amino groups of said dendrimers; and
d) exposing the crosslinked layers to a surface neutralization reaction.

28. The method of claim 27, wherein said first polymer is poly-L-Lysine (PLL).

29. The method of claim 27, wherein said second polymer is poly(glutamic acid) (PGA).

30. The method of claim 27, wherein said layers are crosslinked using 1-Ethyl-3-[3-dimethylaminopropyl]carbodiimide hydrochloride (EDC).

31. The method of claim 27, wherein said neutralization reaction comprises an acetylation reaction.

32. The method of claim 27, wherein said dendrimer is a generation 5 (G5) polyamideamine (PAMAM) or polypropylamine (POPAM) dendrimer.
